(19) United States
(12) Patent Application Publication
(10) Pub. No.: US 2009/0263421 A1
(43) Pub. Date: Oct. 22, 2009

(54) CELLULAR VACCINE

(76) Inventors: Anna-Lena Spetz-Holmgren, Bromma (SE); Ulrika Johansson, Enskede (CH); Jan Anderson, Djursholm (CH); Lilian Walther-jallow, Spanga (CH)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/914,087

(22) PCT Filed: May 10, 2006
(86) PCT No.: PCT/GB06/01709

§ 371 Date: May 7, 2008

Related U.S. Application Data
(60) Provisional application No. 60/679,229, filed on May 10, 2005.

(51) Int. Cl. A61K 39/145; A61K 45/00; A61K 39/12; A61K 39/29; A61K 39/205; A61K 39/275; A61K 39/02; A61K 39/118; A61K 39/095; A61K 39/102; A61P 37/04

(52) U.S. Cl. 424/209.1; 424/93.71; 424/204.1; 424/225.1; 424/226.1; 424/227.1; 424/224.1; 424/232.1; 424/234.1; 424/263.1; 424/249.1; 424/256.1

(57) ABSTRACT

The present invention provides a cellular vaccine for therapeutic or prophylactic treatment of a pathological condition, the vaccine comprising or consisting of a population of CD 4⁺ T cells modified such that they contain an antigenic component, and/or a nucleic acid molecule encoding an antigenic component thereof, wherein the T cells are (a) activated, or capable of being activated, and (b) apoptotic, or capable or being made apoptotic. The invention further provides an adjuvant composition for use in a method of vaccination, the composition comprising or consisting of a population of T cells, wherein the T cells are (a) activated, or capable of being activated, and (b) apoptotic, or capable or being made apoptotic. In addition, the invention provides a composition having microbicide activity, or capable thereof upon exposure to antigen-presenting cells, the composition comprising or consisting of a population of T cells, wherein the T cells are (a) activated, or capable of being activated, and (b) apoptotic, or capable or being made apoptotic. Also provided by the present invention are methods for making and using the vaccines and compositions described herein.

### FIELD OF INVENTION

The present invention relates to T cell compositions, in particular vaccines, adjuvant compositions for use therewith and microbicide compositions. Specifically, the invention provides compositions comprising activated, apoptotic T cells (optionally modified to contain or express a foreign antigen) and the use thereof to provide an activation/maturation signal to antigen-presenting cells and/or to form an anti-microbial milieu.

### INTRODUCTION

Since HIV-1 was identified almost 20 years ago, 20 million people have died from AIDS and more than 40 million are living with HIV-1 today. An estimated three million are under 15 years of age. In addition, more than 13 million children that are currently under age 15 have lost one or both parents to AIDS, most of them in sub-Saharan Africa (source UNAIDS). Africa is currently the worst affected continent but the epidemic is rapidly spreading both in Asia and Latin America. Moreover, disappointing results from the first phase III HIV-1 vaccine trial were announced in February 2003 by the company VaxGen, who has developed a gp120 protein based vaccine. It will take considerable time before the second generation of protective vaccines will have completed their phase III trials.

During development, apoptosis is an inconspicuous process in vivo due to rapid clearance of dead cells by phagocytosing cells, which does not normally evoke immune responses (Henson et al., 2001, Nat Rev Mol Cell Biol 2:627). The phagocytosing antigen-presenting cells, hence, require additional stimulation apart from uptake of apoptotic bodies, per se, to obtain capacity to induce primary T cell activation. It has however become clear that some antigen-presenting cells can acquire antigens from dead infected cells to be presented to virus specific CD8⁺ T cells (Albert et al., 1998, Nature 392:86; Subklewe et al., 2001, J Exp Med 193:405; Arrode et al., 2000, J Virol 74:10018; Larsson et al., 2002, Aids 16:1319; Zhao et al., 2002, J Virol 76:3007). The phenomenon of antigen presentation on MHC class I molecules after exogenous uptake of antigen (cross-presentation) was first described by Bevan who showed that cell associated antigens (minor histocompatibility antigens) can be acquired by bone marrow derived antigen-presenting cells to initiate cytotoxic T cell responses (reviewed in den Haan et al., 2001, Curr Opin Immunol 13:437).

Dendritic cells (DCs) are potent antigen-presenting cells that have the capacity to stimulate lymph-node-based naïve T helper (Th) cells and initiate primary T cell responses (Banchereau et al., 2000, Annu Rev Immunol 18:767-811). It is now generally accepted that immature DCs, residing in peripheral tissues, require activation/maturation signals in order to undergo phenotypic and functional changes to acquire a fully competent antigen-presenting capacity. Activation/maturation of DCs involves several steps such as a transient increased capacity to take up antigen, migration towards nearby lymph nodes and simultaneous up regulation of molecules including chemokine receptors and co-stimulatory molecules. In the lymph node, the DCs provide Th cells with antigen specific “signal 1” and co-stimulatory “signal 2”. Emerging data also support the involvement of a third signal contributing to the polarisation towards Th1 or Th2 responses (Sporri & Reis e Sousa, 2005, Nat Immunol 6:163-70).

Dendritic cells play an important role inducing adaptive immune responses against viruses (Banchereau & Steinman, 1998, Nature 392:245). It has been demonstrated previously that EBV-, HIV-1- and oncogenic-DNA present in apoptotic bodies can be transferred to antigen-presenting cells and subsequently be expressed within the antigen-presenting cell (Holmgren et al., 1999, Blood 93:3956; Spetz et al., 1999, J Immunol 163:736; Bergsmedh et al., 2001, Proc. Natl Acad Sci USA 98:6407; Bergsmedh et al., 2002, Cancer Res 62:575). It was demonstrated that HIV-1 DNA was efficiently transferred to DCs after uptake of apoptotic bodies (Spetz et al., 1999, J Immunol 163:736).

U.S. Pat. No. 6,506,596 describes a method of transfer of genomic DNA from apoptotic bodies to engulfing cells. The engulfing cells are antigen-presenting cells that will synthesise, process and present the proteins on their surface for stimulation or tolerisation of T cells. The method is useful in several pharmaceutical applications, such as vaccine preparations and gene identification procedures.

U.S. Pat. No. 6,602,709 relates to methods for delivering antigens to dendritic cells which are then useful for inducing antigen-specific cytotoxic T lymphocytes and T helper cells. The method comprises contacting dendritic cells capable of internalising antigens for presentation to immune cells with apoptotic cells comprising the antigen that is to be presented by the immune cells.

The present invention seeks to provide improved vaccines, for example for immunisation against HIV infection, and adjuvant compositions and microbicide compositions for use therewith.

### SUMMARY OF INVENTION

A first aspect of the present invention provides an a cellular vaccine for therapeutic or prophylactic treatment of a pathological condition, the vaccine comprising or consisting of a population of CD 4⁺ T cells modified such that they contain an antigenic component and/or a nucleic acid molecule encoding an antigenic component, wherein the T cells are (a) activated, or capable of being activated, and (b) apoptotic, or capable or being made apoptotic.

By “cellular vaccine” we mean a vaccine composition comprising or consisting of CD 4⁺ T cells, which composition is capable of providing a prophylactic and/or therapeutic treatment effect against a pathological condition when administered into a suitable subject. In particular, in the context of the present invention, the cellular vaccine is capable of providing active immunisation in a host against a pathological condition.

The cellular vaccines of the invention are believed to provide an activation/maturation signal to immature antigen-presenting cells, thus enabling effective antigen presentation after uptake and processing of antigen, leading to induction of immune responses.

It will be appreciated by persons skilled in the art that the cellular vaccines of the invention need not be 100% pure. For example, the vaccines may comprise CD 4⁺ T cells which are not activated and/or are not induced to undergo apoptosis, or capable of the same. In addition, the vaccines may additionally comprise cells other than T cells, such as monocytes (e.g. low CD4⁺ expressing monocytes). In one embodiment, however, the cellular vaccines of the invention are predominantly composed of CD 4⁺ T cells which are (a) activated, or capable of being activated, and (b) apoptotic, or capable or being made apoptotic, for example at least 20%, 30%, 40%, 50%, 60%, 70%, 80%, 90%, 95%, 96%, 97%, 98% or 99% or more of such T cells (i.e. % by number of T cells to total number of all cell types). In a further preferred embodiment, in particular for cellular vaccines against HIV, the vaccine is substantially free of CD 8⁺ T cells (e.g. less than 5%, for example 4%, 3%, 2%, 1% or less CD 8⁺ T cells, and most preferably completely free of CD 8⁺ T cells).

By ‘treatment’ we include both therapeutic and prophylactic treatment of the subject/patient. The term ‘prophylactic’ is used to encompass the use of a composition described herein which either prevents or reduces the likelihood of a pathologic condition developing in a patient or subject. For example, the composition may provide partial or complete protection against the pathologic condition in a patient or subject by inducing production in the patient or subject of antibodies against a pathogen. The term ‘therapeutic’ is used to encompass the use of a composition described herein which induces a favourable change in a pathologic condition in a patient or subject, whether that change is a remission, a favourable physiological result, a reversal or attenuation of a disease state or condition treated, depending upon the disease or condition treated.

By “pathological condition” we include disease states of the human and animal body. For example, the pathological condition may be a disease or condition caused by the infection or infestation of a host with a pathogenic microbial agent, such as a virus, bacterium, protozoa, mycoplasma, yeast or fungus. In addition, the term “pathological condition” is intended to include other disease states of the human and animal body, such as proliferative disorders (i.e. cancers).

By “T cells” we mean T cell receptor bearing (T-) lymphocytes. Likewise, by “CD 4⁺ T cells” we mean T-lymphocytes which express on their surface the CD4 glycoprotein (CD 4⁺ T cells are also known as T helper cells). Similarly, by “CD 8⁺ T cells” we mean T-lymphocytes which express on their surface the CD8 glycoprotein (CD 8⁺ T cells are also known as cytotoxic T cells).

By “modified” we mean that the T cells are genetically engineered, conjugated, fused, derivatised or otherwise altered from their natural state such that they contain an antigenic component and/or a nucleic acid encoding such an antigenic component. Preferably, the modified T cells display the antigenic component at their surface. In particular, as used herein, the term ‘modified’ includes the modification of a T cell through introduction of foreign DNA, such as but not limited to microbial genes, by using an appropriate method. Preferably, microbial genes are introduced through transfection or infection, but also other methods, such as fusion, can be used.

By “antigenic component” we include foreign (i.e. non-T cell derived) proteins, carbohydrates and lipids, and combinations and fragments thereof, which are capable of inducing the immune system to make a specific immune response. Thus, in the context of viruses, the term ‘antigenic component’ specifically encompasses whole virions, proteins (such as, but not limited to, envelope and capsid proteins), carbohydrates and lipids derived therefrom, as well as combinations thereof, and fragments of the same which are capable of eliciting an immune response in a host. Likewise, the term ‘antigenic component’ also encompasses components, such as proteins, carbohydrates and lipids, as well as combinations and fragments thereof, derived from bacterial cells, which components are capable of eliciting an immune response in a host. In addition, in the context of cancer cells, the term ‘antigenic component’ specifically encompasses cell surface expressed proteins, and antigenic fragments thereof, associated (either exclusively or preferentially) with cancer cells. Also included within the scope of the term ‘antigenic component’ as used herein are variant, i.e. non-naturally occurring, forms of naturally-occurring antigenic components, such as variant proteins or fragments thereof which have been mutated to enhance their antigenic potential. It will be appreciated by skilled persons that the antigenic component, such as a protein or lipid, may comprise carbohydrate moieties; for example, the antigenic component may be a glycoprotein or glycolipid, or fragment thereof.

By “activated”, in the context of T cells, as used herein, we include the modification of a large number of T cell proteins by exposure to a suitable activating agent (i.e. activation produces a recognisable phenotypic change in the T cells). Activation of the T cells can be confirmed by studying, for example, T cell proliferation and upregulation of CD69, CD25 and CD40L. Examples of T cell-activating mediators include, but not limited to, lectins (such as PHA and ConA), chemicals or agents that induce Ca²⁺ influx in the T cells (such as ionomycin), alloantigens, superantigens (which interact with the T cell receptor in a domain outside of the antigen recognition site, such as Staphylococcal enterotoxins A and B [SEA and SEB]), monoclonal antibodies (such as anti-CD3, anti-CD28 and anti-CD49d, used either alone or in combination), cytokines (such as IL-1 and TNF-α), chemokines and chemokine receptors, and molecules capable of interfering with T cell surface receptors or their signal transducing molecules. The phrase ‘capable of being activated’ shall be construed accordingly. It will be appreciated by skilled persons that activation of T cells may be induced either in vitro or in vivo.

Certain activating agents, such as PHA, ConA and superantigens, require the presence of antigen-presenting cells (APCs) in order to activate the T cells. Hence, in one embodiment the cellular vaccine (or other compositions of the invention; see below) may comprise or consist of peripheral blood mononuclear cells (PBMCs), containing both T cells and monocytes (as APCs). In a further embodiment, the PBMCs are treated to remove CD8+ cells but preserve the monocytes, to allow enhanced activation (and, optionally, inclusion of virus variants). Optionally, the monocytes are cultured with a maturation stimulus prior to use, for example IL-4 and GM-CSF.

By “apoptotic” we mean programmed cell death in which the T cells ultimately disintegrate into membrane-bound particles which are then eliminated by phagocytosis. Apoptosis may be induced by exposure of the T cells to an apoptosis-inducing agent, such as gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids. The phrase ‘capable or being made apoptotic’ shall be construed accordingly. As in the case of activation, it will be appreciated by skilled persons that apoptosis of T cells may be induced either in vitro or in vivo.

In a preferred embodiment of the first aspect of the invention, the CD 4⁺ T cells are obtainable or obtained by a method comprising:
(a) activating a population of CD 4⁺ T cells;
(b) modifying the population of CD 4⁺ T cells such that they contain an antigenic component, and/or a nucleic acid molecule encoding an antigenic component; and
(c) inducing the population of CD 4⁺ T cells to undergo apoptosis
   wherein steps (a) to (c) may be performed in any order.

Advantageously, the method further comprises culturing the population of CD 4⁺ T cells in an appropriate medium. Culturing of the T cells may be performed at any stage of the above process, for example before or after activation and/or modification of the T cells.

The term “appropriate medium”, as used herein, refers to any medium that can be used for culturing T cells, thus enabling the cells to grow and divide. Examples of such media include, but are not limited to, Ex vivo 15, Ex vivo 10, AIM V, LGM1, 2 or 3, Stemline, RPMI containing 2 mM L-glutamine, 1% penicillin-streptomycin, 10 mM HEPES, 5-10% serum (autologous serum), human AB+ serum and foetal calf serum. Ex vivo media may be used without addition of serum. The cells can be cultured with or without addition of IL-2 and/or IL-7 to the medium.

Conveniently, the method further comprises freezing the population of CD 4⁺ T cells. This optional step may be performed at any stage of the above process, for example before or after activation and/or modification of the T cells. Preferably, the cells are frozen after activation and modification, and then stored until the time of use (apoptosis may be induced either prior to freezing or after the cells have been thawed ready for use).

The CD 4⁺ T cells may be obtained from any suitable source using methods well known in the art. For example, the T cells may be obtained from peripheral blood mononuclear cells (PBMCs) isolated from a blood sample.

Preferably, the CD 4⁺ T cells are isolated/derived from primary lymphocytes. The T cells may be enriched for cells expressing the CD 4⁺ glycoprotein either by positive selection for CD 4⁺ T cells or by negative selection (i.e. depletion) of CD 8⁺ T cells. Suitable methods are well known in the art.

For example, T cells may be isolated by methods such as immunomagnetic isolation, Sheep red blood cell rosette formation with or without inclusion of an antibody-based separation step, flow cytometry based cell sorting, leukapheresis methods, density gradients, antibody panning methods, and antibody/complement depletion (see also Current Protocols in Immunology, 2006, by John Wiley & sons, Editors; Coligan, Bierer, Margulies, Shevach, Strober and Coico; Hami et al., 2004, Cytotherapy 6:554-62).

It will be appreciated by persons skilled in the art that the CD 4⁺ T cells may be derived from human or non-human animals, e.g. domestic and farm animals (including mammals such as dogs, cats, horses, cows, sheep, etc.).

Preferably, however, the CD 4⁺ T cells are derived from a human.

In a preferred embodiment, the CD 4⁺ T cells are derived from the subject in which the cellular vaccine is to be used, i.e. the T cells are autologous.

In an alternative embodiment, the CD 4⁺ T cells are derived from the same species as that of the subject in which the cellular vaccine is to be used, i.e. the T cells are allogeneic.

An essential feature of the cellular vaccine of the first aspect of the invention is that the CD 4⁺ T cells are activated, or capable of being activated. Preferably, the CD 4⁺ T cells are activated, or capable of being activated, by exposure to an activating agent selected from the group consisting of lectins (such as PHA and ConA), chemicals or agents that induce Ca²⁺ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

The concentration and exposure time required for each activating agent can be determined by routine experimentation.

Preferably, the activating agent is PHA. For example, the T cells (together with monocytes/APCs) may be cultured overnight or longer in medium containing 2.5 μg/ml PHA.

Alternatively, the activating agent may be one or more monoclonal antibodies (for example, at a concentration in the medium of 2 μg/ml). Particularly preferred monoclonal antibody activating agents include anti-CD3 antibodies, anti-CD28 antibodies and anti-CD49d antibodies, used either alone or in combination.

In the cellular vaccine aspect of the present invention, the CD 4⁺ T cells are modified such that they contain an antigenic component, or a nucleic acid molecule encoding an antigenic component. However, it will be appreciated by skilled persons that it is not essential for all the T cells in the vaccine to be so modified; thus, the vaccine may comprise a mixture of modified and non-modified T cells.

In one embodiment, the CD 4⁺ T cells are modified such that they contain a microorganism or antigenic component thereof, or a nucleic acid molecule encoding a microorganism or antigenic component thereof. Preferably, the microorganism is selected from the group consisting of bacteria, mycoplasmas, protozoa, yeasts, prions, archaea, fungi and viruses.

Preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism is a bacterium. Thus, the CD 4⁺ T cells may be modified such that they contain an antigenic component of a bacterial cell, or a nucleic acid molecule encoding such an antigenic component. For example, the bacterium may be selected from the group consisting of Mycobacterium tuberculosis, salmonella, listeria, Treponema pallidum, Neisseria gonorrhoeae, Chlamydia trachomatis and Haemophilus ducreyi.

In one embodiment, the microorganism is a protozoan, such as the causative agent of malaria (i.e. Plasmodium falciparum, Plasmodium vivax, Plasmodium ovale, or Plasmodium malariae) or Trichomonas vaginalis.

In a further preferred embodiment, the CD 4⁺ T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell-associated antigens include those listed in Table 1 below.

TABLE 1
Tumour Associated Antigens

| Antigen | Antibody | Existing Uses |
|---|---|---|
| Carcino-embryonic | C46 (Amersham) | Imaging & Therapy of |
| Antigen | 85A12 (Unipath) | colon/rectum tumours. |
| Placental Alkaline | H17E2 (ICRF, | Imaging & Therapy of |
| Phosphatase | Travers & Bodmer) | testicular and ovarian |
| | | cancers. |
| Pan Carcinoma | NR-LU-10 (NeoRx | Imaging & Therapy of |
| | Corporation) | various carcinomas incl. |
| | | small cell lung cancer. |
| Polymorphic | HMFG1 (Taylor- | Imaging & Therapy of |
| Epithelial Mucin | Papadimitriou, ICRF) | ovarian cancer, pleural |
| (Human milk fat | (Antisoma plc) | effusions, breast, lung |
| globule | | & other common |
| | | epithelial cancers. |
| Human milk mucin | SM-3(IgG1)¹ | Diagnosis, Imaging |
| core protein | | & Therapy of breast |
| | | cancer |
| β-human Chorionic | W14 | Targeting of enzyme |
| Gonadotropin | | (CPG2) to human |
| | | xenograft |
| | | choriocarcinoma in |
| | | nude mice. (Searle et al |
| | | (1981) Br. J. Cancer |
| | | 44, 137-144) |
| A Carbohydrate on | L6 (IgG2a)² | Targeting of alkaline |
| Human Carcinomas | | phosphatase. (Senter et |
| | | al (1988) Proc. Natl. |
| | | Acad. Sci. USA 85, |
| | | 4842-4846 |
| CD20 Antigen on B | 1F5 (IgG2a)³ | Targeting of alkaline |
| Lymphoma (normal | | phosphatase. (Senter et |
| and neoplastic) | | al (1988) Proc. Natl. |
| | | Acad. Sci. USA 85, |
| | | 4842-4846 |

¹Burchell et al (1987) Cancer Res. 47, 5476-5482
²Hellström et al (1986) Cancer Res. 46, 3917-3923
³Clarke et al (1985) Proc. Natl. Acad. Sci. USA 82, 1766-1770

Other suitable cancer cell-associated antigens include alphafoetoprotein, Ca-125, prostate specific antigen and members of the epidermal growth factor receptor family, namely EGFR, erbB2, erbB3 and erbB4.

A further essential feature of the cellular vaccine of the first aspect of the invention is that the CD 4⁺ T cells are apoptotic, or capable or being made apoptotic by exposure to an apoptosis-inducing agent. For example, the apoptosis-inducing agent may be selected from the group consisting of gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids.

Preferably, the apoptosis-inducing agent is gamma-irradiation.

It will be appreciated by persons skilled in the art that cells may be treated such that they will undergo apoptosis in vivo (i.e. after administration into the subject being treated with the vaccine). For example, the cells may be injected shortly after treatment with an agent that will induce apoptosis (e.g. 30 min to 2 hrs after apoptosis induction), without an in vitro step. Hence, at the time of injection, the apoptotic machinery may have been initiated but apoptosis not yet induced. In other words, the cells may undergo apoptosis in vivo after being injected.

The activated, apoptotic CD 4⁺ T cells in the cellular vaccine are capable of activation/maturation of antigen-presenting cells.

The terms “activation of antigen-presenting cells” and “maturation of antigen-presenting cells”, as used herein, refer to the activation/maturation of antigen-presenting cells, such as dentritic cells (DCs) through the addition of a signal initiating such activation/maturation. Antigen-presenting cells require activation/maturation signals in order to undergo phenotypic and functional changes to acquire a fully competent antigen-presenting capacity. Activation/maturation of, for example, DCs involves several steps such as a transient increased capacity to take up antigen, migration towards nearby lymph nodes and simultaneous up regulation of molecules including chemokine receptors and co-stimulatory molecules.

Examples of activation/maturation signals include, but not limited to, inflammatory mediators such as cytokines (TNF-α), CD40 ligand, microbial and viral products (pathogen-associated molecular patterns, PAMPs). PAMP are recognised by pattern-recognition receptors (PRRs) including members of the Toll-like receptor (TLR) family. PRR signalling in DCs leads to production of pro-inflammatory cytokines such as interferon-α (IFN-α) or IFN-β, tumour necrosis factor-α (TNF α) and interleukin-1 (IL-1), which can also promote DC activation steps. One embodiment of the present invention encompasses induction of activation/maturation in antigen-presenting cells by using apoptotic, activated T cells.

In one embodiment, the activated, apoptotic CD 4⁺ T cells in the cellular vaccine of the invention induce activation/maturation of endogenous antigen-presenting cells in the host being treated with the vaccine.

In an alternative embodiment of the first aspect of the invention, the cellular vaccine further comprises a population of (exogenous) antigen-presenting cells. Preferably, the antigen-presenting cells are macrophages and/or dendritic cells.

Thus, the invention encompasses the possibility of isolating APCs, e.g. dendritic cells, from a patient (and/or deriving them in vitro from a patient's monocytes), inducing APC maturation in vitro with the cellular vaccine and then injecting the matured APCs into the patient.

A second aspect of the present invention provides a pharmaceutical composition comprising a cellular vaccine according to the first aspect of the invention and a pharmaceutically acceptable carrier or diluent.

Examples of suitable pharmaceutical compositions and routes of administration thereof are described in detail below.

Preferably, however, the pharmaceutical composition is suitable for parenteral administration.

An additional aspect of the present invention provides a kit of parts for preparing a cellular vaccine according to the first aspect of the invention, the kit comprising or consisting of:

(a) a population of modified CD 4⁺ T cells, or means of obtaining the same;
(b) an activating agent; and
(c) an apoptosis-inducing agent.

A fourth aspect of the present invention provides a method for making a cellular vaccine according to the first aspect of the invention, the method comprising:
(a) obtaining a population of CD 4⁺ T cells; and
(b) modifying the CD 4⁺ T cells such that they contain an antigenic component, and/or a nucleic acid molecule encoding an antigenic component
   wherein the T cells are activated (or capable of being activated) and apoptotic (or capable or being made apoptotic).

It will be appreciated that the modification, activation and induction (e.g. initiation) of apoptosis of the T cells are performed in vitro.

Advantageously, step (a) comprises isolating/purifying the CD 4⁺ T cells from primary lymphocytes (as described above).

Preferably, the population of CD 4⁺ T cells in step (a) are derived from the subject in which the cellular vaccine is to be used, i.e. the T cells are autologous.

Alternatively, the population of CD 4⁺ T cells in step (a) may be derived from the same species as that of the subject in which the cellular vaccine is to be used, i.e. the T cell are allogeneic.

In one embodiment, step (b) comprises modifying the CD 4⁺ T cells such that they contain a microorganism or antigenic component thereof, or a nucleic acid molecule encoding a microorganism or antigenic component thereof. Preferably, the microorganism is selected from the group consisting of bacteria, mycoplasmas, protozoa, prions, archaea, yeasts, fungi and viruses.

Preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism may be a bacterium. Thus, the CD 4⁺ T cells may be modified such that they contain an antigenic component of a bacterial cell, or a nucleic acid molecule encoding such an antigenic component. For example, the bacterium may be selected from the group consisting of Mycobacterium tuberculosis, salmonella, listeria, Treponema pallidum, Neisseria gonorrhoeae, Chlamydia trachomatis and Haemophilus ducreyi.

In one embodiment, the microorganism is a protozoan, such as the causative agent of malaria (i.e. Plasmodium falciparum, Plasmodium vivax, Plasmodium ovale, or Plasmodium malariae) or Trichomonas vaginalis.

In a further preferred embodiment, step (b) comprises modifying the CD 4⁺ T cells such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell associated antigens include those listed in Table 1 above.

Modification of the CD 4⁺ T cells may be accomplished using techniques well known in the art, for example transfection, infection and fusion.

The term “transfection”, as used herein, refers to the introduction of foreign DNA into the T cell, through the use of a vector, such as, but not limited to, a virus, phage, plasmid or synthetic carrier of DNA (e.g. a nanoparticle). Transfection can also be accomplished through electrical stimulation.

The term “infection”, as used herein, refers to colonisation of a host organism by a foreign species. The colonising organism interferes with the normal functioning and, eventually perhaps, the survival of the host. The infecting organism is referred to as a pathogen. Examples of pathogens include, but not limited to, bacteria, parasites, fungi and viruses.

The term “fusion”, as used herein, refers to a method for introducing foreign DNA into a T cell through the fusion with another cell comprising the DNA to be transferred. In order to fuse two cells, the cell membranes need to be permeabilised. Permeabilisation can be obtained, for example, through the addition of a detergent, such as, but not limited to, poly ethylene glycol (PEG). Mixing the two cell types in the presence of a detergent will make it possible for the two cell types to fuse. In one embodiment of the present invention a cell comprising microbial DNA is fused with an activated T cell according to the invention and thereby introduces the foreign DNA into the immunostimulatory T cell. Alternatively, a pathogen that does not normally infect the activated T cell may be transferred into the cell by fusion using a reagent such as PEG.

Thus, in a preferred embodiment of the fourth aspect of the invention, the CD 4⁺ T cells are modified by transfection with a nucleic acid molecule encoding the antigen component. For example, the nucleic acid molecule may be a viral or bacterial gene encoding an antigenic protein or fragment thereof, or alternatively may be a gene encoding a cancer cell-associated antigen or fragment thereof same.

In a particularly preferred embodiment, transfection is achieved using nanoparticles to which are coupled nucleic acid molecules encoding the antigenic component (see Examples). Alternatively, the nanoparticles may be coupled directly to the antigenic component itself.

Alternatively, the CD 4⁺ T cells are modified by infection with a whole virus/virion.

In a preferred embodiment of the fourth aspect of the invention, the method further comprises the step of activating the CD 4⁺ T cells (either before or after modification of the T cells; see above).

For example, the CD 4⁺ T cells may be activated by exposure to an activating agent selected from the group consisting of lectins (such as PHA and ConA), chemicals or agents that induce Ca2+ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

Optionally, the method of the fourth aspect of the invention further comprises the step of culturing the CD 4⁺ T cells (at any stage of the method).

Conveniently, the method also comprises freezing the population of CD 4⁺ T cells. This optional step may be performed at any stage of the above process, for example before or after activation and/or modification of the T cells. Preferably, the cells are frozen after activation and modification, and then stored until the time of use (apoptosis may be induced either prior to freezing or after the cells have been thawed ready for use).

In a further preferred embodiment of the fourth aspect of the invention, the method additionally comprises the step of inducing the CD 4⁺ T cells to undergo apoptosis (either before or after activation and/or modification of the T cells; see above).

For example, apoptosis may be induced by exposure to an apoptosis-inducing agent selected from the group consisting of selected among gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids.

Persons skilled in the art will appreciate that the order in which the steps of the fourth aspect of the invention are performed is arbitrary. However, the steps are preferably performed in one of the following orders:
(a) activation, culturing (optional), modification, freezing (optional) and induction of apoptosis;
(b) culturing (optional), activation, modification, freezing (optional) and induction of apoptosis;
(c) activation, culturing (optional), modification, induction of apoptosis and freezing (optional);
(d) modification, culturing (optional), activation, freezing (optional) and induction of apoptosis; or
(e) modification, culturing (optional), activation, induction of apoptosis and freezing (optional).

In yet another preferred embodiment of the fourth aspect of the invention, the method additionally comprises the step of the step of adding a population of antigen-presenting cells to the cellular vaccine.

Advantageously, the antigen-presenting cells are macrophages or dendritic cells (see above).

In a particularly preferred embodiment of the fourth aspect of the invention, the method is suitable for GMP-production of a cellular HIV vaccine according to the invention, the method comprising the following steps, in order:
(a) peripheral blood mononuclear cells (PBMCs) are isolated from a blood sample from the patient to be tested;
(b) the PBMCs isolated in step (a) are enriched for CD4+ cells (e.g. the CD8+ cells are depleted from the PBMCs);
(c) the CD4+ cell-enriched cells obtained in step (b) are cultured in vitro;
(d) the cells are activated (for example, with anti-CD8 and anti-CD28 mAbs in the presence of IL-2);
(e) the supernatant is collected to provide an HIV virus stock from the patient;
(f) the obtained virus stock is stored frozen;
(g) steps (a) and (b) are repeated to prepare the cells to be used as immunogens;
(h) the cells obtained in step (g) are cultured in vitro;
(i) the cells are activated (for example, with anti-CD8 and anti-CD28 mAbs in the presence of IL-2);
(j) the activated CD8 negative PBMCs are incubated with autologous virus, from the stock obtained in step (f), to obtain infected cells;
(k) on the day of immunisation of the patient, the infected cells are thawed (if frozen), washed and exposed to an apoptosis-inducing agent (for example, gamma-irradiation); and
(l) the cells are kept in room temperature after apoptosis induction and are used for immunisation within 2 hours.

In Step (e), the virus stock can be ultracentrifuged to get an even higher concentration of the virus. The virus stock (or bank) can also be investigated to measure the titre using TCID50 tests and/or p24 ELISA. Sterility in terms of other pathogens can also be investigated.

In Step (j), the obtained infected cells can be stored frozen. An aliquot of the autologous infected cell stock (or bank) can be analysed for sterility, mycoplasma, endotoxin, HIV-DNA content, HIV-RNA content, HIV-p24 protein content, % CD4/CD8 cells, and % T cell activation markers (such as CD69 and CD25) by flow cytometry.

In Step (j), an aliquot of the cells can be analysed for efficacy of apoptosis induction, which may be measured after incubation in vitro. An aliquot can also used to investigate the capacity to mature DCs in vitro (e.g. upregulation of co-stimulatory molecules).

A fifth aspect of the present invention provides a method for treatment of a subject with a pathological condition, the method comprising administering to the subject a cellular vaccine according to the first aspect of the invention or a pharmaceutical composition according to the second aspect of the invention.

It will be appreciated by persons skilled in the art that the subject may be human or a non-human animal, e.g. domestic and farm animals (including mammals such as dogs, cats, horses, cows, sheep, etc.). Preferably, however, the subject is human.

In a preferred embodiment, the pathological condition is caused by a microorganism selected from the group consisting of bacteria, mycoplasmas, protozoa, prions, archaea, yeasts, fungi and viruses.

For example, the pathological condition may be caused by a virus. Exemplary viruses include, but are not limited to, retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

In a particularly preferred embodiment, the virus is an HIV virus, such as HIV1 or HIV2.

Alternatively, the pathological condition may caused by bacteria, for example selected from the group consisting of Mycobacterium tuberculosis, salmonella, listeria, Treponema pallidum, Neisseria gonorrhoeae, Chlamydia trachomatis and Haemophilus ducreyi.

In a further embodiment, the pathological condition may be caused by protozoan, such as the causative agent of malaria (i.e. Plasmodium falciparum, Plasmodium vivax, Plasmodium ovale, or Plasmodium malariae) or Trichomonas vaginalis.

In a further preferred embodiment, the CD 4⁺ T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Conveniently, the T cells in the cellular vaccine are exposed to an apoptosis-inducing agent immediately prior to (e.g. within 2 hours of) administration to the subject.

A sixth aspect of the invention provides a cellular vaccine according to the first aspect of the invention or a pharmaceutical composition according to the second aspect of the invention for use in medicine, for example in the treatment of a subject with a pathological condition.

A seventh aspect of the invention provides the use of a cellular vaccine according to the first aspect of the invention or a pharmaceutical composition according to the second aspect of the invention in the preparation of a medicament for treatment of a subject with a pathological condition.

Exemplary pathological conditions are described above.

An eighth aspect of the invention provides an adjuvant composition for use in a method of vaccination, the composition comprising or consisting of a population of T cells, wherein the T cells are (a) activated, or capable of being activated, and (b) apoptotic, or capable or being made apoptotic.

By “adjuvant composition” we mean a composition which is capable of enhancing the immunogenicity of an antigen. In the context of the present invention, the ‘adjuvant composition’ is capable of augmenting the adaptive immunity induced by administration of a vaccine to a subject. In particular, this aspect of the invention provides a population of T cells capable of delivering an activation/maturation signal to antigen-presenting cells.

The concept of “adjuvant compositions” is described in detail in Gamvrellis et al. (2004) Immunology & Cell Biology 82:506-516.

Typically, the adjuvant composition and the vaccine are separate entities. However, it will be appreciated by persons skilled in the art that the adjuvant composition and the vaccine may be a single entity (see below).

It will also be appreciated by persons skilled in the art that the adjuvant composition may comprise CD 4⁺ T cells and/or CD 8⁺ T cells. For example, the adjuvant composition may comprise or consist of PBMCs.

In an alternative embodiment, the adjuvant composition comprises preferentially or predominantly CD 4⁺ T cells, for example at least 50%, 60%, 70%, 80%, 90%, 95%, 96%, 97%, 98%, 99% or more CD 4⁺ T cells.

In an alternative embodiment, the adjuvant composition comprises preferentially or predominantly CD 8⁺ T cells, for example at least 50%, 60%, 70%, 80%, 90%, 95%, 96%, 97%, 98%, 99% or more CD 8+T cells.

The T cells for use in the adjuvant compositions of the invention may be obtained from any suitable source, using methods well known in the art. For example, the T cells may be obtained from peripheral blood mononuclear cells (PBMCs) isolated from a sample blood.

Preferably, the T cells are isolated/derived from primary lymphocytes. The T cells may be enriched for cells expressing the CD 4⁺ or CD 8+T glycoproteins either by positive selection for or by negative selection (i.e. depletion) of a subpopulation of T cells. Suitable methods are well known in the art.

For example, T cells may be isolated by methods such as immunomagnetic isolation, Sheep red blood cell rosette formation with or without inclusion of an antibody-based separation step, flow cytometry based cell sorting, leukapheresis methods, density gradients, antibody panning methods, and antibody/complement depletion (see also Current Protocols in Immunology, 2006, by John Wiley & sons, Editors; Coligan, Bierer, Margulies, Shevach, Strober and Coico; Hami et al., 2004, Cytotherapy 6:554-62).

It will be appreciated by persons skilled in the art that the T cells may be derived from human or non-human animals, e.g. domestic and farm animals (including mammals such as dogs, cats, horses, cows, sheep, etc.). Preferably, however, the T cells are derived from a human source.

In a preferred embodiment, the T cells are derived from the subject in which the adjuvant composition is to be used, i.e. the T cells are autologous.

In an alternative embodiment, the T cells are derived from the same species as that of the subject in which the adjuvant composition is to be used, i.e. the T cells are allogeneic.

In a preferred embodiment of the eighth aspect of the invention, the T cells are activated, or capable of being activated, by exposure to an activating agent selected from the group consisting of lectins (such as PHA and ConA), chemicals or agents that induce Ca²⁺ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

The concentration and exposure time required for each activating agent can be determined by routine experimentation.

Preferably, the activating agent is PHA. For example, the T cells (together with monocytes/APCs) may be cultured overnight or longer in medium containing 2.5 μg/ml PHA.

Alternatively, the activating agent may be one or more monoclonal antibodies (for example, at a concentration in the medium of 2 μg/ml). Particularly preferred monoclonal antibody activating agents include anti-CD3 antibodies, anti-CD28 antibodies and anti-CD49d antibodies, used either alone or in combination.

A further essential feature of the adjuvant composition of the eighth aspect of the invention is that the T cells are apoptotic, or capable or being made apoptotic by exposure to an apoptosis-inducing agent. For example, the apoptosis-inducing agent may be selected from the group consisting of gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids.

Preferably, the apoptosis-inducing agent is gamma-irradiation.

It will be appreciated by persons skilled in the art that cells may be treated such that they will undergo apoptosis in vivo (i.e. after administration into the subject being treated with the vaccine). For example, the cells may be injected shortly after treatment with an agent that will induce apoptosis (e.g. 30 min to 2 hrs after apoptosis induction), without an in vitro step. Hence, at the time of injection, the apoptotic machinery may have been initiated but apoptosis not yet induced. In other words, the cells may undergo apoptosis in vivo after being injected.

Thus, the activated, apoptotic T cells in the adjuvant composition are capable of activation/maturation of antigen-presenting cells.

It will be further appreciated by persons skilled in the art that the adjuvant compositions of the present invention are suitable for use with any vaccine which provides active immunisation.

Advantageously, the adjuvant composition is for use with a vaccine against a pathogenic condition selected from the group consisting of HIV, tuberculosis, malaria, influenza and cancer.

Preferably, the vaccine is an HIV vaccine.

Alternatively, the vaccine may be a cancer vaccine.

The adjuvant composition may be used in conjunction with any vaccine capable of presenting an antigen to the host immune system. For example, the vaccine may comprise or consist of an attenuated or original viral vector selected from the group consisting of adenoviruses (such as adenoviruses 1, 2 and 5, chimpanzee), hepatitis viruses (such as hepatitis B virus and hepatitis C virus), Pox viruses (such as canarypox, vaccinia), rabies virus, murine leukaemia virus, alpha replicons, measles, rubella, polio, calicivirus, paramyxovirus, vesicular stomatitis virus, papilloma, leporipox, parvovirus, papovavirus, togavirus, picornavirus, reovirusx and ortmyxovirus (such as influenza viruses) and bacterial vectors (such as vectors selected from the group of mycobacteria, salmonella, listeria, Treponema pallidum, Neisseria gonorrhoeae, Chlamydia trachomatis and Haemophilus ducreyi).

In one embodiment of the adjuvant compositions of the eighth aspect of the invention, the T cells are modified such that they contain an antigenic component, and/or a nucleic acid molecule encoding an antigenic component thereof.

For example, the T cells may be modified such that they contain a microorganism or antigenic component thereof, or a nucleic acid molecule encoding a microorganism or antigenic component thereof. Preferably, the microorganism is selected from the group consisting of bacteria, mycoplasmas, protozoa, yeasts, prions, archaea, fungi and viruses.

More preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism may be a bacterium. Thus, the CD 4⁺ T cells may be modified such that they contain an antigenic component of a bacterial cell, or a nucleic acid molecule encoding such an antigenic component. For example, the bacteria may be selected from the group consisting of Mycobacterium tuberculosis, salmonella, listeria, Treponema pallidum, Neisseria gonorrhoeae, Chlamydia trachomatis and Haemophilus ducreyi.

In one embodiment, the microorganism is a protozoan, such as the causative agent of malaria (i.e. Plasmodium falciparum, Plasmodium vivax, Plasmodium ovale, or Plasmodium malariae) or Trichomonas vaginalis.

In a further preferred embodiment, the T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell associated antigens include those listed in Table 1 above.

In one embodiment, the activated, apoptotic T cells in the adjuvant composition of the invention induce activation/maturation of endogenous antigen-presenting cells in the host in being treated with the adjuvant composition.

In an alternative embodiment of the eighth aspect of the invention, the adjuvant composition further comprises a population of (exogenous) antigen-presenting cells. Preferably, the antigen-presenting cells are macrophages and/or dendritic cells.

Conveniently, the composition is frozen, for storage prior to use.

A ninth aspect of the present invention provides a pharmaceutical composition comprising an adjuvant composition according to the eighth aspect of the invention and a pharmaceutically acceptable carrier or diluent.

Examples of suitable pharmaceutical compositions and routes of administration thereof are described in detail below.

Preferably, however, the pharmaceutical composition is suitable for parenteral administration.

The present invention further provides, as a tenth aspect, a combination product comprising:

(a) an adjuvant composition according to the eighth aspect of the invention; and
(b) a vaccine,
wherein each of components (a) and (b) is formulated in admixture with a pharmaceutically-acceptable diluent or carrier.

In a preferred embodiment the combination product of the invention comprises an adjuvant composition according to the eighth aspect of the invention, a vaccine and a pharmaceutically-acceptable diluent or carrier.

In an alternative embodiment, the combination product of the invention comprises a kit of parts comprising components:
(a) a pharmaceutical formulation according to the ninth aspect of the invention; and
(b) a vaccine;
   which components (a) and (b) are each provided in a form that is suitable for administration in conjunction with the other.

By bringing the two components “into association with” each other, we include that components (a) and (b) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, in respect of the combination product according to the invention, the term “administration in conjunction with” includes that the two components of the combination product (i.e. a pharmaceutical formulation according to the ninth aspect of the invention and a vaccine) are administered (optionally repeatedly), either together, or sufficiently closely in time, to enable a beneficial effect for the patient. Determination of whether a combination provides a beneficial effect in respect of, and over the course of treatment of, a particular condition will depend upon the condition to be treated or prevented, but may be achieved routinely by the skilled person.

An additional aspect of the present invention provides a kit of parts for preparing an adjuvant composition according to the eighth aspect of the invention, the kit comprising or consisting of;

(a) a population of T cells, or means of obtaining the same;
(b) an activating agent; and
(c) an apoptosis-inducing agent.

A twelfth aspect of the present invention provides a method for making an adjuvant composition according to the eighth aspect of the invention, the method comprising obtaining a population of T cells, wherein the T cells are activated (or capable of being activated) and apoptotic (or capable or being made apoptotic).

Advantageously, the T cells are isolated/purified from primary lymphocytes (as described above).

Preferably, the population of T cells is derived from the subject in which the adjuvant composition is to be used, i.e. the T cells are autologous.

Alternatively, the population of T cells may be derived from the same species as that of the subject in which the adjuvant composition is to be used, i.e. the T cells are allogeneic.

In a preferred embodiment of the twelfth aspect of the invention, the method further comprises the step of activating the T cells (either before or after modification of the T cells; see above).

For example, the T cells may be activated by exposure to an activating agent selected from the group consisting of lectins (such as PHA and ConA), chemicals or agents that induce Ca²⁺ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

Preferably, the activating agent is PHA. For example, the T cells (together with monocytes/APCs) may be cultured overnight or longer in medium containing 2.5 μg/ml PHA.

Alternatively, the activating agent may be one or more monoclonal antibodies (for example, at a concentration in the medium of 2 μg/ml). Particularly preferred monoclonal antibody activating agents include anti-CD3 antibodies, anti-CD28 antibodies and anti-CD49d antibodies, used either alone or in combination.

Optionally, the method of the twelfth aspect of the invention further comprises the step of culturing the T cells (at any stage of the method).

Conveniently, the method also comprises freezing the population of T cells. This optional step may be performed at any stage of the above process, for example before or after activation and/or modification of the T cells. Preferably, the cells are frozen after activation and modification, and then stored until the time of use (apoptosis may be induced wither prior to freezing after the cells have been thawed ready for use).

In a further preferred embodiment of the twelfth aspect of the invention, the method additionally comprises the step of inducing the T cells to undergo apoptosis (either before or after activation and/or modification of the T cells; see above).

For example, apoptosis may be induced by exposure to an apoptosis-inducing agent selected from the group consisting of selected among gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids. The phrase ‘capable or being made apoptotic’ shall be construed accordingly.

In one embodiment of the twelfth aspect of the invention, the T cells are modified such that they contain an antigenic component thereof, or a nucleic acid molecule encoding an antigenic component.

For example, step (b) may comprise modifying the T cells such that they contain a microorganism or antigenic component thereof, or a nucleic acid molecule encoding a microorganism or antigenic component thereof. Preferably, the microorganism is selected from the group consisting of bacteria, mycoplasmas, protozoa, prions, archaea, yeasts, fingi and viruses.

Preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism may be a bacterium. Thus, the T cells may be modified such that they contain an antigenic component of a bacterial cell, or a nucleic acid molecule encoding such an antigenic component. For example, the bacterium may be selected from the group consisting of Mycobacterium tuberculosis, salmonella, listeria, Treponema pallidum, Neisseria gonorrhoeae, Chlamydia trachomatis and Haemophilus ducreyi.

In another embodiment, the microorganism is a protozoan, such as the causative agent of malaria (i.e. Plasmodium falciparum, Plasmodium vivax, Plasmodium ovale, or Plasmodium malariae) or Trichomonas vaginalis.

In a further preferred embodiment of the twelfth aspect of the invention, the T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell-associated antigens include those listed in Table 1 above.

Modification of the T cells may be accomplished using techniques well known in the art, for example transfection, infection and fusion (see above).

In a particularly preferred embodiment, transfection is achieved using nanoparticles to which are coupled nucleic acid molecules encoding the antigenic component. Alternatively, the nanoparticles may be coupled directly to the antigenic component itself.

Alternatively, the T cells may be modified by infection with a whole virus/virion.

Optionally, the method of the twelfth aspect of the invention further comprises the step of culturing the T cells (at any stage of the method).

Conveniently, the method also comprises freezing the population of T cells. This optional step may be performed at any stage of the above process, for example before or after activation and/or modification of the T cells. Preferably, the cells are frozen after activation and modification, and then stored until the time of use (apoptosis may be induced wither prior to freezing after the cells have been thawed ready for use).

Persons skilled in the art will appreciate that the order in which the steps of the twelfth aspect of the invention are performed is arbitrary. However, the steps are preferably performed in one of the following orders:
(a) activation, culturing (optional), modification (optional), freezing (optional) and induction of apoptosis;
(b) culturing (optional), activation, modification (optional), freezing (optional) and induction of apoptosis;
(c) activation, culturing (optional), modification (optional), induction of apoptosis and freezing (optional);
(d) modification (optional), culturing (optional), activation, freezing (optional) and induction of apoptosis; or
(e) modification (optional), culturing (optional), activation, induction of apoptosis and freezing (optional).

In yet another preferred embodiment of the twelfth aspect of the invention, the method additionally comprises the step of adding a population of antigen-presenting cells to the adjuvant composition.

Advantageously, the antigen-presenting cells are macrophages or dendritic cells (see above).

A thirteenth aspect of the invention provides a method for treatment of a subject with a pathological condition, the method comprising administering to the subject a vaccine together with an adjuvant composition according to the eighth aspect of the invention, a pharmaceutical composition according to the ninth aspect of the invention, or a combination product according to the tenth aspect of the invention.

It will be appreciated by persons skilled in the art that the subject may be human or a non-human animal, e.g. domestic and farm animals (including mammals such as dogs, cats, horses, cows, sheep, etc.). Preferably, however, the subject is human.

It will also be appreciated by skilled persons that the vaccine and adjuvant composition can be distinct agents or a single agent. For example, in the latter case, the adjuvant composition may comprise activated, apoptotic T cells modified to contain an antigenic component.

In a preferred embodiment, the thirteenth aspect of the invention provides a method of vaccination.

In one embodiment, the pathological condition is caused by a microorganism selected from the group consisting of bacteria, mycoplasmas, yeasts, prions, archaea, fungi and viruses.

For example, the pathological condition may be caused by a virus. Exemplary viruses include, but are not limited to, group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

In a particularly preferred embodiment, the virus is an HIV virus, such as HIV1 or HIV2.

Alternatively, the pathological condition may be caused by a bacterium, for example selected from the group consisting of Mycobacterium tuberculosis, salmonella, listeria, Treponema pallidum, Neisseria gonorrhoeae, Chlamydia trachomatis and Haemophilus ducreyi.

In a further embodiment, the pathological condition may be caused by a protozoan, such as the causative agent of malaria (i.e. Plasmodium falciparum, Plasmodium vivax, Plasmodium ovale, or Plasmodium malariae) or Trichomonas vaginalis.

In a further preferred embodiment, the T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Conveniently, the T cells in the adjuvant composition are exposed to an apoptosis-inducing agent immediately prior to (e.g. within 2 hours of) administration to the subject.

A thirteenth aspect of the invention provides an adjuvant composition according to the eighth aspect of the invention, a pharmaceutical composition according to the ninth aspect of the invention, or a combination product according to the tenth aspect of the invention for use in medicine, for example in the treatment of a subject with a pathological condition.

A fourteenth aspect of the invention provides the use of an adjuvant composition according to the eighth aspect of the invention, a pharmaceutical composition according to the ninth aspect of the invention, or a combination product according to the tenth aspect of the invention in the preparation of a medicament for treatment of a subject with a pathological condition.

Exemplary pathological conditions are described above.

Related aspects of the invention further provide:
(i) a method of enhancing the effect of a vaccine comprising administering to a subject an adjuvant composition according to the eighth aspect of the invention, a pharmaceutical composition according to the ninth aspect of the invention, or a combination product according to the tenth aspect of the invention.
(ii) a method of activating antigen-presenting cells comprising contacting the antigen-presenting cells with an adjuvant composition according to the eighth aspect of the invention, a pharmaceutical composition according to the ninth aspect of the invention, or a combination product according to the tenth aspect of the invention. Thus, there is provided a method for delivering an activation and maturation signal to antigen-presenting cells.

It will be appreciated that the above methods may be performed in vivo or in vitro.

A fifteenth aspect of the invention provides a composition having microbicide activity, or capable thereof upon exposure to antigen-presenting cells, the composition comprising or consisting of a population of T cells, wherein the T cells are (a) activated, or capable of being activated, and (b) apoptotic, or capable or being made apoptotic.

By a “composition having microbicide activity” we mean that the composition which is able, at least in part, to kill or inhibit the growth and/or prevent infection of one or more microorganism species (for example, viruses, bacteria, etc.), or is capable of killing or inhibiting the growth or preventing infection thereof upon exposure of the composition to antigen-presenting cells.

Thus, the invention provides a composition which is capable of producing a microbicide milieu in combination with antigen-presenting cells. This effect may be achieved in vivo or in vitro.

It will be appreciated by persons skilled in the art that the microbicide composition may comprise CD 4⁺ T cells and/or CD 8+T cells. For example, the microbicide composition may comprise or consist of PBMCs.

In an alternative embodiment, the microbicide composition comprises predominantly CD 4⁺ T cells, for example at least 50%, 60%, 70%, 80%, 90%, 95%, 96%, 97%, 98%, 99% or more CD 4⁺ T cells.

In an alternative embodiment, the microbicide composition comprises predominantly CD 8⁺ T cells, for example at least 50%, 60%, 70%, 80%, 90%, 95%, 96%, 97%, 98%, 99% or more CD 8+T cells.

The T cells for use in the microbicide compositions of the invention may be obtained from any suitable source, using methods well known in the art. For example, the T cells may be obtained from peripheral blood mononuclear cells (PBMCs) isolated from a sample blood.

Alternatively, the T cells may be obtained or derived from an immortalised cell line.

Preferably, the T cells are isolated/derived from primary lymphocytes. The T cells may be enriched for cells expressing the CD 4⁺ or CD 8⁺ T glycoproteins either by positive selection for or by negative selection (i.e. depletion) of a subpopulation of T cells. Suitable methods are well known in the art.

For example, T cells may be isolated by methods such as immunomagnetic isolation, Sheep red blood cell rosette formation with or without inclusion of an antibody-based separation step, flow cytometry based cell sorting, leukapheresis methods, density gradients, antibody panning methods, and antibody/complement depletion (see also Current Protocols in Immunology, 2006, by John Wiley & sons, Editors; Coligan, Bierer, Margulies, Shevach, Strober and Coico; Hami et al., 2004, Cytotherapy 6:554-62).

It will be appreciated by persons skilled in the art that the T cells may be derived from human or non-human animals, e.g. domestic and farm animals (including mammals such as dogs, cats, horses, cows, sheep, etc.).

Preferably, however, the T cells may be derived from a human.

In a preferred embodiment, the T cells are derived from the subject in which the microbicide composition is to be used, i.e. the T cells are autologous.

In an alternative embodiment, the T cells are derived from the same species as that of the subject in which the microbicide composition is to be used, i.e. the T cells are allogeneic.

In a preferred embodiment of the fifteenth aspect of the invention, the T cells are activated, or capable of being activated, by exposure to an activating agent selected from the group consisting of lectins (such as PHA and ConA), chemicals or agents that induce Ca²⁺ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

The concentration and exposure time required for each activating agent can be determined by routine experimentation.

Preferably, the activating agent is PHA. For example, the T cells (together with monocytes/APCs) may be cultured overnight or longer in medium containing 2.5 μg/ml PHA.

Alternatively, the activating agent may be one or more monoclonal antibodies (for example, at a concentration in the medium of 2 μg/ml). Particularly preferred monoclonal antibody activating agents include anti-CD3 antibodies, anti-CD28 antibodies and anti-CD49d antibodies, used either alone or in combination.

A further essential feature of the composition of the fifteenth aspect of the invention is that the T cells are apoptotic, or capable or being made apoptotic by exposure to an apoptosis-inducing agent. For example, the apoptosis-inducing agent may be selected from the group consisting of gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids.

Preferably, the apoptosis-inducing agent is gamma-irradiation.

It will be appreciated by persons skilled in the art that cells are treated in a way that they will undergo apoptosis in vivo (i.e. after administration into the subject being treated with the microbicide). For example, the cells may be injected shortly after treatment with an agent that will induce apoptosis (e.g. 30 min to 2 hrs after apoptosis induction), without an in vitro step. Hence, at the time of injection, the apoptotic machinery may have been initiated but apoptosis not yet induced. In other words, the cells may undergo apoptosis in vivo after being injected.

Thus, the activated, apoptotic T cells in the microbicide composition are capable of activation/maturation of antigen-presenting cells. Activation/maturation of antigen-presenting cells is known to make them less susceptible to HIV-1 infection (see McDyer et al., 1999, J. Immunology 162:3711-3717).

In one embodiment of the microbicide compositions of the fifteenth aspect of the invention, the T cells are modified such that they contain an antigenic component, and/or a nucleic acid molecule encoding an antigenic component thereof.

For example, the T cells may be modified such that they contain a microorganism or antigenic component thereof, or a nucleic acid molecule encoding a microorganism or antigenic component thereof. Preferably, the microorganism is selected from the group consisting of bacteria, mycoplasmas, protozoa, yeasts, prions, archaea, fungi and viruses.

More preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism may be a bacterium. Thus, the T cells may be modified such that they contain an antigenic component of a bacterial cell, or a nucleic acid molecule encoding such an antigenic component. For example, the bacterium may be selected from the group consisting of Mycobacterium tuberculosis, salmonella, listeria, Treponema pallidum, Neisseria gonorrhoeae, Chlamydia trachomastis and Haemophilus ducreyi.

In one embodiment, the microorganism is a protozoan, such as the causative agent of malaria (i.e. Plasmodium falciparum, Plasmodium vivax, Plasmodium ovale, or Plasmodium mailariae) or Trichomonas vaginalis.

In a further preferred embodiment, the T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell associated antigens include those listed in Table 1 above.

In one embodiment, the activated, apoptotic T cells in the microbicide composition of the invention induce activation/maturation of endogenous antigen-presenting cells in the host.

In an alternative embodiment of the fifteenth aspect of the invention, the microbicide composition further comprises a population of (exogenous) antigen-presenting cells. Preferably, the antigen-presenting cells are macrophages and/or dendritic cells.

Conveniently, the composition is frozen, for storage prior to use.

Advantageously, the microbicide composition according to the fifteenth aspect of the invention further comprises a pharmaceutically acceptable carrier or diluent (i.e. a pharmaceutical composition).

Examples of suitable pharmaceutical compositions and routes of administration thereof are described in detail below.

Preferably, the pharmaceutical composition is suitable for local mucosal administration prior to or after exposure to a pathogen.

Conveniently, the pharmaceutical composition is suitable for parenteral administration.

The present invention further provides, as a sixteenth aspect, a combination product comprising:

(a) a composition according to the fifteenth aspect of the invention; and
(b) a population of antigen-presenting cells,
wherein each of components (a) and (b) is formulated in admixture with a pharmaceutically-acceptable diluent or carrier.

In a preferred embodiment, the combination product of the invention comprises a microbicide composition according to the fifteenth aspect of the invention, a population of antigen-presenting cells and a pharmaceutically-acceptable diluent or carrier.

In an alternative embodiment, the combination product of the invention comprises a kit of parts comprising components:
(a) a pharmaceutical composition according to the fifteenth aspect of the invention; and
(b) a population of antigen-presenting cells,
   which components (a) and (b) are each provided in a form that is suitable for administration in conjunction with the other.

By bringing the two components “into association with” each other, we include that components (a) and (b) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, in respect of the combination product according to the invention, the term “administration in conjunction with” includes that the two components of the combination product (i.e. a pharmaceutical formulation according to the fifteenth aspect of the invention and a population of antigen-presenting cells) are administered (optionally repeatedly), either together, or sufficiently closely in time, to enable a beneficial effect for the patient. Determination of whether a combination provides a beneficial effect in respect of, and over the course of treatment of, a particular condition will depend upon the condition to be treated or prevented, but may be achieved routinely by the skilled person.

An additional aspect of the present invention provides a kit of parts for preparing a composition according to the fifteenth aspect of the invention, the kit comprising or consisting;

(a) a population of T cells, or means of obtaining the same;
(b) an activating agent; and
(c) an apoptosis-inducing agent.

An eighteenth aspect of the invention provides a method of making a composition according to the fifteenth aspect of the invention, the method comprising obtaining a population of T cells, wherein the T cells are activated (or capable of being activated) and apoptotic (or capable or being made apoptotic).

Advantageously, the T cells are isolated/purified from primary lymphocytes (as described above).

Preferably, the population of T cells is derived from the subject in which the microbicide composition is to be used, i.e. the T cells are autologous.

Alternatively, the population of T cells may be derived from the same species as that of the subject in which the microbicide composition is to be used, i.e. the T cells are allogeneic.

In a preferred embodiment of the eighteenth aspect of the invention, the method further comprises the step of activating the T cells (either before or after modification of the T cells; see above).

For example, the T cells may be activated by exposure to an activating agent selected from the group consisting of lectins (such as PHA and ConA), chemicals or agents that induce Ca²⁺ influx in the T cells (such as ionomycin), alloantigens, superantigens (such as SEA and SEB), monoclonal antibodies (such as anti-CD3, anti-CD28 and anti-CD49d), cytokines (such as IL-1 and TNF-α, chemokine and chemokine receptors, and molecules capable of interfering with T cell surface receptors or their signal transducing molecules.

Preferably, the activating agent is PHA. For example, the T cells (together with monocytes/APCs) may be cultured overnight or longer in medium containing 2.5 μg/ml PHA.

Alternatively, the activating agent may be one or more monoclonal antibodies (for example, at a concentration in the medium of 2 μg/ml). Particularly preferred monoclonal antibody activating agents include anti-CD3 antibodies, anti-CD28 antibodies and anti-CD49d antibodies, used either alone or in combination.

Optionally, the method of the eighteenth aspect of the invention further comprises the step of culturing the T cells (at any stage of the method).

Conveniently, the method also comprises freezing the population of T cells. This optional step may be performed at any stage of the above process, for example before or after activation and/or modification of the T cells. Preferably, the cells are frozen after activation and modification, and then stored until the time of use (apoptosis may be induced wither prior to freezing after the cells have been thawed ready for use).

In a further preferred embodiment of the eighteenth aspect of the invention, the method additionally comprises the step of inducing the T cells to undergo apoptosis (either before or after activation and/or modification of the T cells; see above).

For example, apoptosis may be induced by exposure to an apoptosis-inducing agent selected from the group consisting of gamma-irradiation, cytostatic drugs, UV-irradiation, mitomycin C, starvation (e.g. serum deprivation), Fas ligation, cytokines and activators of cell death receptors (as well as their signal transducing molecules), growth factors (and their signal transducing molecules), interference with cyclins, over-expression of oncogenes, molecules interfering with anti-apoptotic molecules, interference of the membrane potential of the mitochondria and steroids.

In one embodiment of the eighteenth aspect of the invention, the T cells are modified such that they contain an antigenic component thereof, or a nucleic acid molecule encoding an antigenic component.

For example, step (b) may comprise modifying the T cells such that they contain a microorganism or antigenic component thereof, or a nucleic acid molecule encoding a microorganism or antigenic component thereof. Preferably, the microorganism is selected from the group consisting of bacteria, mycoplasmas, protozoa, yeasts, prions, archaea, fungi and viruses.

Preferably, the microorganism is a virus. For example, the virus may be selected from the group consisting of retroviruses (such as HIV viruses, e.g. HIV1 and HIV2), adenoviruses (such as adenoviruses 1, 2 and 5, chimpanzee), hepatitis viruses (such as hepatitis B virus and hepatitis C virus), CMV, Epstein-Barr virus (EBV), herpes viruses (such as HHV6, HHV7 and HHV8), human T-cell lymphotropic viruses (such as HTLV1 and HTLV2), Pox viruses (such as canarypox, vaccinia), rabies viruses, murine leukaemia viruses, alpha replicons, measles, rubella, polio, caliciviruses, paramyxoviruses, vesicular stomatitis viruses, papilloma, leporipox, parvoviruses, papovaviruses, togaviruses, picornaviruses, reoviruses and ortmyxoviruses (such as influenza viruses).

Most preferably, the virus is an HIV virus, such as HIV1 or HIV2.

Alternatively, the microorganism may be a bacterium. Thus, the T cells may be modified such that they contain an antigenic component of a bacterial cell, or a nucleic acid molecule encoding such an antigenic component. For example, the bacterium may be selected from the group consisting of Mycobacterium tuberculosis, salmonella, listeria, Treponema pallidum, Neisseria gonorrhoeae, Chlamydia trachomatis and Haemophilus ducreyi.

In another embodiment, the microorganism is a protozoan, such as the causative agent of malaria (i.e. Plasmodium falciparum, Plasmodium vivax, Plasmodium ovale, or Plasmodium malariae) or Trichomonas vaginalis.

In a further preferred embodiment of the eighteenth aspect of the invention, the T cells are modified such that they contain an antigenic component of a cancer cell, or a nucleic acid molecule encoding such an antigenic component. Preferably, the cancer cell is selected from the group consisting of cancer cells of the breast, bile duct, brain, colon, stomach, bone, reproductive organs, lung and airways, skin, gallbladder, liver, nasopharynx, nerve cells, kidney, prostate, lymph glands, gastrointestinal tract, bone marrow, blood and other tumour cells containing viruses.

Examples of such cancer cell associated antigens include those listed in Table 1 above.

Modification of the T cells may be accomplished using techniques well known in the art, for example transfection, infection and fusion (see above).

In a particularly preferred embodiment, transfection is achieved using nanoparticles to which are coupled nucleic acid molecules encoding the antigenic component. Alternatively, the nanoparticles may be coupled directly to the antigenic component itself.

Alternatively, the T cells may be modified by infection with a whole virus/virion.

Optionally, the method of the eighteenth aspect of the invention further comprises the step of culturing the T cells (at any stage of the method).

Conveniently, the method also comprises freezing the population of T cells. This optional step may be performed at any stage of the above process, for example before or after activation and/or modification of the T cells. Preferably, the cells are frozen after activation and modification, and then stored until the time of use (apoptosis may be induced wither prior to freezing after the cells have been thawed ready for use).

Persons skilled in the art will appreciate that the order in which the steps of the eighteenth aspect of the invention are performed is arbitrary. However, the steps are preferably performed in one of the following orders:
(a) activation, culturing (optional), modification (optional), freezing (optional) and induction of apoptosis;
(b) culturing (optional), activation, modification (optional), freezing (optional) and induction of apoptosis;
(c) activation, culturing (optional), modification (optional), induction of apoptosis and freezing (optional);
(d) modification (optional), culturing (optional), activation, freezing (optional) and induction of apoptosis; or
(e) modification (optional), culturing (optional), activation, induction of apoptosis and freezing (optional).

In yet another preferred embodiment of the eighteenth aspect of the invention, the method additionally comprises the step of adding a population of antigen-presenting cells to the microbicide composition.

Advantageously, the antigen-presenting cells are macrophages or dendritic cells (see above).

A nineteenth aspect of the invention provides a method for treatment of a subject with a pathological condition, or recently exposed to a pathogen or susceptible to such exposure, the method comprising administering to the subject a composition according to the fifteenth aspect of the invention, or a combination product according to the sixteenth aspect of the invention.

It will be appreciated by persons skilled in the art that the subject may be human or a non-human animal, e.g. domestic and farm animals (including mammals such as dogs, cats, horses, cows, sheep, etc.). Preferably, however, the subject is human.

In one embodiment, the pathological condition is caused by a microorganism selected from the group consisting of bacteria, mycoplasmas, yeasts, fungi, prions, archaea and viruses.

For example, the pathological condition may be caused by a virus (i.e. the pathogen to which subject has been or could be exposed may be a virus). Exemplary viruses include, but are not limited to, retroviruses (such as HIV viruses, e.g. HIV1 and HIV2), herpes simplex viruses, human papilloma viruses, and Leporipox viruses.

In a particularly preferred embodiment, the virus is an HIV virus, such as HIV1 or HIV2.

Alternatively, the pathological condition may be caused by a bacterium, for example selected from the group consisting of Treponema pallidum, Neisseria gonorrhoeae, Chlamydia trachomatis and Haemophilus ducreyi.

In a further embodiment, the pathological condition may be caused by a protozoan (such as Trichomonas vaginalis) or fungus (such as Candida albicans).

A twentieth aspect of the invention provides a composition according to the fifteenth aspect of the invention or a combination product according to the sixteenth aspect of the invention for use in medicine, for example in the treatment of a subject with a pathological condition or after expose to a pathogen.

A twenty-first aspect of the invention provides the use of composition according to the fifteenth aspect of the invention or a combination product according to the sixteenth aspect of the invention in the preparation of a medicament for treatment of a subject with a pathological condition or after expose to a pathogen.

Exemplary pathological conditions are described above.

Related aspects of the invention further provide:
(i) a method for making a composition having microbicide activity, the composition comprising contacting a population of activated, apoptotic T cells with a population of antigen-presenting cells in a cell medium in vitro and then obtaining cell medium therefrom (e.g. as a supernatant).
(ii) a composition having microbicide activity obtained or obtainable by the above method (preferably, comprising one or more chemokines/cytokines with anti-viral activity).

Several of the above-mentioned aspects of the invention constitute pharmaceutical compositions, for example comprising a cellular vaccine, adjuvant composition or microbicide composition according to the invention.

It will be appreciated by persons skilled in the art that such an effective amount of the vaccines and compositions of the invention may be delivered as a single bolus dose (i.e. acute administration) or, more preferably, as a series of doses over time (i.e. chronic administration).

The vaccines and compositions of the invention can be formulated at various concentrations, depending on the efficacy/toxicity of the compound being used and the indication for which it is being used. Preferably, the formulation comprises an amount of the vaccine or composition of the invention comprising about 0.1-100×10⁶ cells.

It will be appreciated by persons skilled in the art that the cellular vaccines, adjuvant compositions or microbicide compositions of the invention will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA).

For example, the agents of the invention can be administered orally, buccally or sublingually in the form of tablets, capsules, ovules, elixirs, solutions or suspensions, which may contain flavouring or colouring agents, for immediate-, delayed- or controlled-release applications. The agents of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropyhnethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The agents of the invention can also be administered parenterally, for example, intravenously, intra-nasally, intra-dermally, locally applied to the vagina, mouth or rectum, intra-articularly, intra-arterially, intraperitoneally, intra-thecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For mucosal (e.g. oral or vaginal) and parenteral administration to human patients, the daily dosage level of the agents of the invention will usually be from about 0.1-100×10⁶ cells per adult, administered in single or divided doses.

The agents of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or ‘puff’ contain about 0.1-100×10⁶ cells for delivery to the patient. It will be appreciated that the overall daily dose with an aerosol will vary from patient to patient, and may be administered in a single dose or, more usually, in divided doses throughout the day.

Alternatively, the agents of the invention can be administered in the form of a suppository or pessary, or they may be applied topically in the form of a lotion, solution, cream, ointment or dusting powder. The compounds of the invention may also be transdermally administered, for example, by the use of a skin patch or other intra-dermal devices. They may also be administered by the ocular route.

For application topically to the skin, the agents of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Persons skilled in the art will further appreciate that the agents and pharmaceutical formulations of the present invention have utility in both the medical and veterinary fields. Thus, the agents of the invention may be used in the treatment of both human and non-human animals (such as horses, dogs and cats). Preferably, however, the patient is human.

Preferred aspects of the invention are described in the following non-limiting examples, with reference to the following figures:

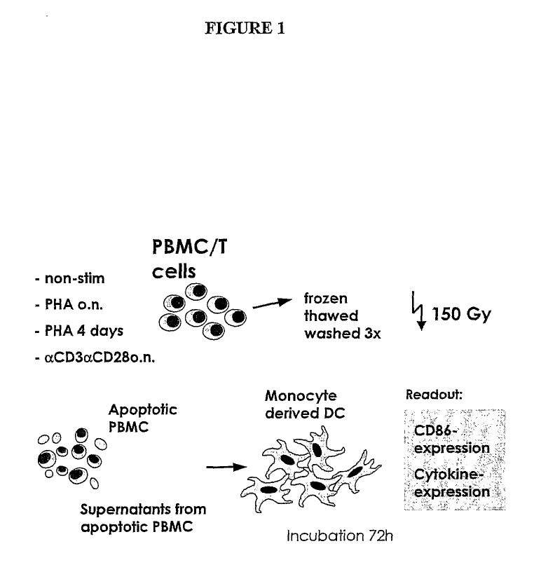

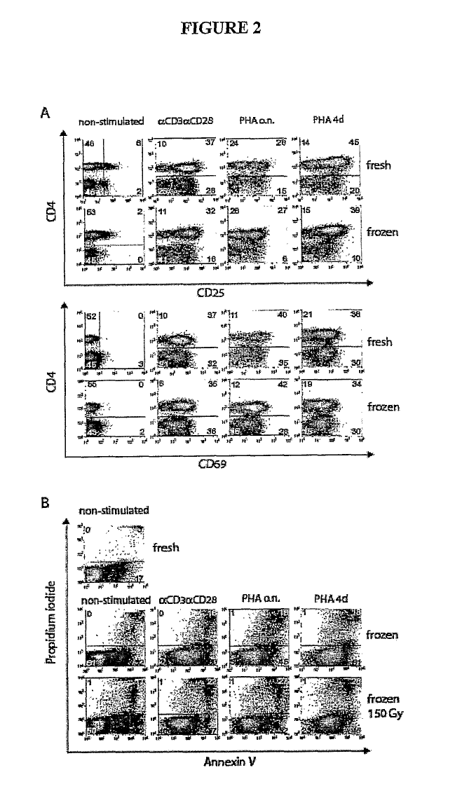

FIG. 1. The figure shows the principle set up in vitro, which comprises induction of apoptosis in autologous or allogeneic cells and thereafter addition to phagocytes. Flow cytometry is used for measurements of apoptosis by annexin V/PI stainings, phenotypic analyses of apoptotic cells, dendritic cell maturation, quantification of phagocytosis, and intracellular cytokine production. PBMC—peripheral blood mononuclear cells.

FIG. 2. Phenotypic characterization of apoptotic activated peripheral blood mononuclear cells (PBMCs)

PBMCs were isolated from healthy blood donors and put in culture without any additional stimulation (non-stimulated), activated with anti-CD3 and anti-CD28 mAbs over night, PHA (phytohemagglutinin) over night or with PHA for 4 days. The cells were either stained directly after the culture period or stained after a freezing period in DMSO. The recovered cells were stained with anti-CD4, anti-CD25 and anti-CD69 mAbs and analysed for surface expression by flow cytometry. Data are shown on gated on lymphocytes. The quadrants are set based on isotype control stainings and the numbers depicts the frequency of cells in each quadrant (A). PBMCs were also analysed for apoptosis induction as defined by Annexin-V and PI staining (B). Freshly isolated non-stimulated PBMCs display the background staining. PBMCs were put in culture without any additional stimulation (non-stimulated), activated with anti-CD3 and anti-CD28 mAbs over night, PHA over night or with PHA for 4 days. Cells were then frozen in DMSO. After thawing the cells were either stained directly with Annexin-V and PI or first exposed to 150Gy gamma-irradiation. Staining with Annexin-V and PI were performed directly after gamma-irradiation.

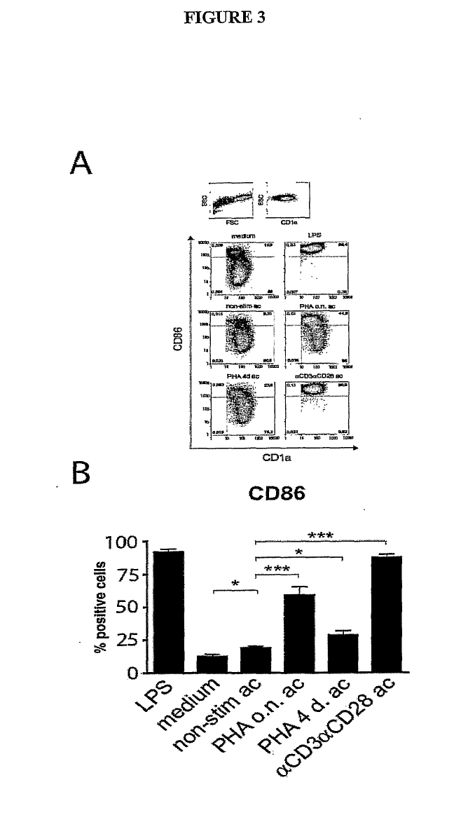

FIG. 3. Apoptotic activated PBMCs induce CD86 expression in human DCs

Human in vitro differentiated monocytes cultured for 6 days in the presence of IL-4 and GM-CSF were used as source of human immature DCs as defined by their expression of CD1a, lack of CD14 and low expression of CD40, CD80, CD86 and CD83. These immature DCs were co-cultured with different apoptotic cells (ac) for 72 hours and then analyzed for expression of CD86 molecules. Gates were set on large CD1a⁺CD3⁻ cells. LPS (lipopolysaccharide), which is a potent DC activator, was used as positive control and DCs cultured in medium only was used as negative control. The apoptotic cells were from freshly isolated PBMCs (non-stim ac), PBMCs activated with PHA over night (PHA o.n. ac), PBMCs activated with PHA for 4 days (PHA 4d ac) or PBMCs activated with anti-CD3 and anti-CD28 mAb over night (αCD3αCD28 ac) (A). Representative flow cytometric analyses and the definition of quadrant settings are shown. The different PBMCs were induced to undergo apoptosis by gamma-irradiation just prior to addition of DCs. (B) Average frequency of CD86 expressing DCs±SD of at least eight experiments. Significant differences were assessed by non-parametric Mann-Whitney test and are indicated by * (P<0.05), ** (P<0.01) and *** (P<0.001), respectively.

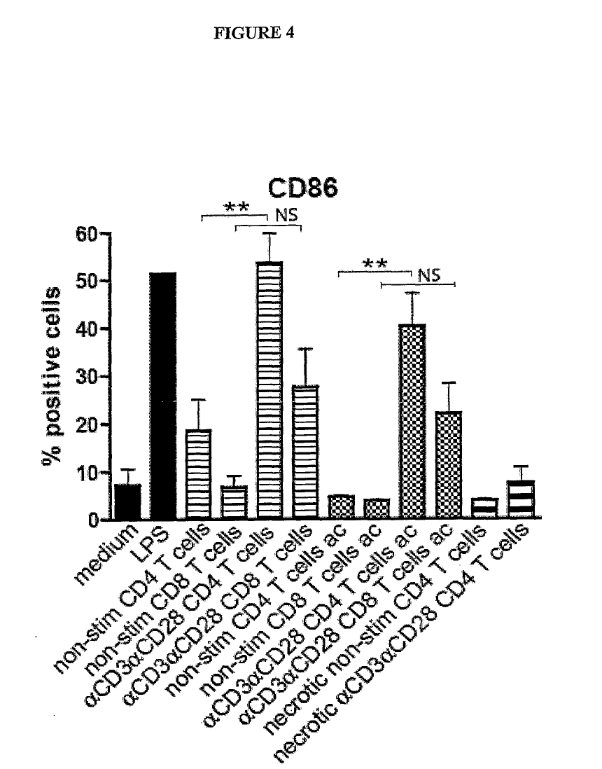

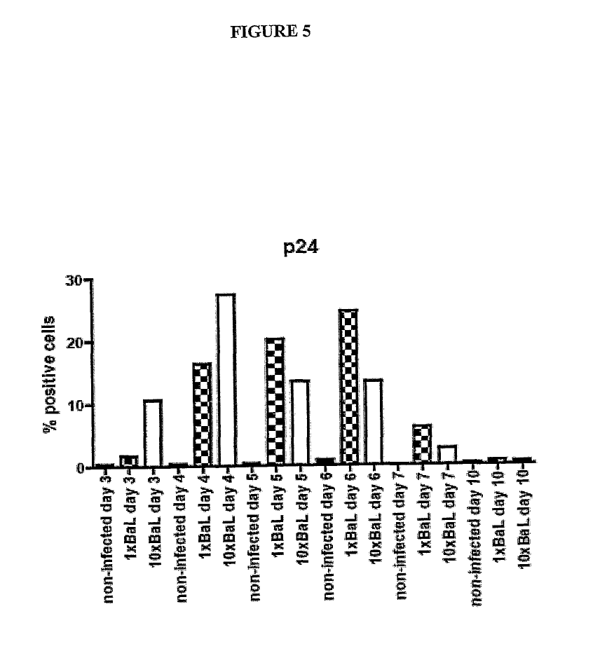

FIG. 4. Apoptotic activated CD4⁺ T cells are efficient inducers of CD86 expression in DCs

Immature DCs were co-cultured with live non-activated or anti-CD3/CD28 activated (over night incubation) CD4⁺ or CD8⁺ T cells isolated by negative depletion. Immature DCs were also co-cultured with apoptotic non-activated or antiCD3/CD28 activated CD4⁺ or CD8⁺ T cells. In addition, necrotic non-activated or antiCD3/CD28 activated CD4⁺ T cells induced to undergo necrosis by repeated freeze thawing cycles were also co-cultured with immature DCs. The expression of CD86 was assessed by flow cytometry after 72 h of co-culture. Gates were set on large CD1a⁺CD3⁻ cells. LPS was used as a positive control and negative control was culture in only medium. Average frequency of CD86 expressing DCs±SD of at least four experiments. Significant differences were assessed by non-parametric Mann-Whitney test and are indicated by * (P<0.05), and ** (P<0.01), respectively.

FIG. 5. HIV-1 infection in anti-CD3 and anti-CD28 activated CD4⁺ T cells

CD4⁺ T cells were activated with anti-CD3 and anti-CD28 mAb over night before they were infected with either 1xBaL stock or a 10xBaL stock. The frequency of infection was measured by intracellular p24 staining and quantified by flow cytometry. The kinetics of infection of one representative experiment is shown.

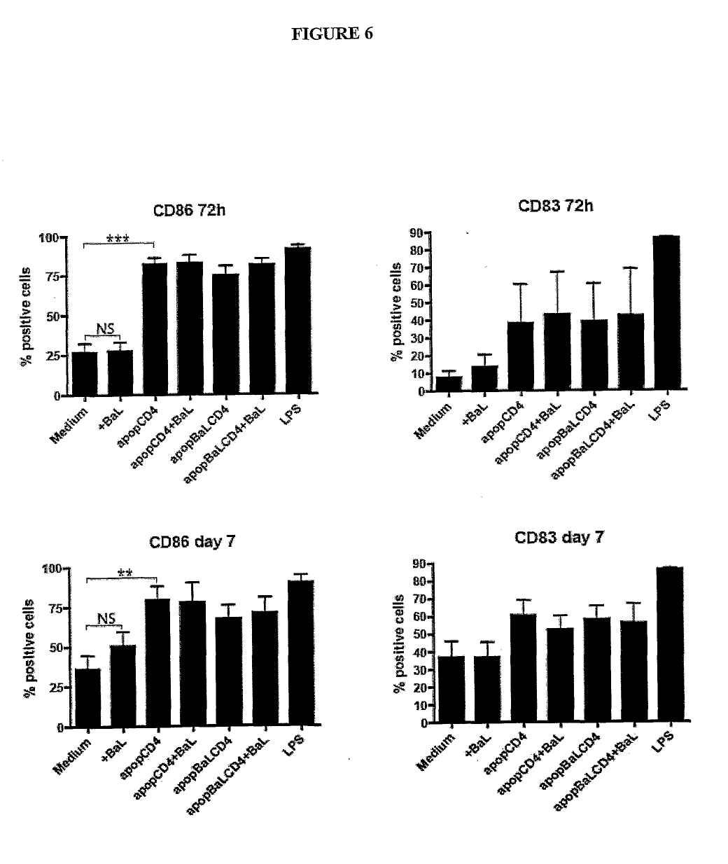

FIG. 6. Apoptotic activated HIV-1 infected cells induce DC activation/maturation

Immature DCs were cultured in medium, in the presence of HIV-1_{BaL} (+BaL), apoptotic anti-CD3 and anti-CD28 activated CD4⁺ T cells (apopCD4), apoptotic anti-CD3 and anti-CD28 activated CD4⁺ T cells in the presence of free HIV-1_{BaL} (apopCD4+BaL), apoptotic activated HIV-1_{BaL} infected CD4⁺ T cells (apopBaLCD4), apoptotic activated HIV-1_{BaL} infected CD4⁺ T cells in the presence of free HIV-1_{BaL} or LPS for 72 hours (top) or 7 days (bottom). The expression of CD86 (left) and CD83 (right) was assessed by flow cytometry. Gates were set on large CD1a⁺CD3⁻ cells. Average frequency of CD86 expressing DCs±SD of at least nine experiments at 72 h and five experiments at 7 days. CD83 expression was examined in two experiments. Significant differences were assessed by non-parametric Mann-Whitney test and are indicated by ** (P<0.01) and *** (P<0.001), respectively.

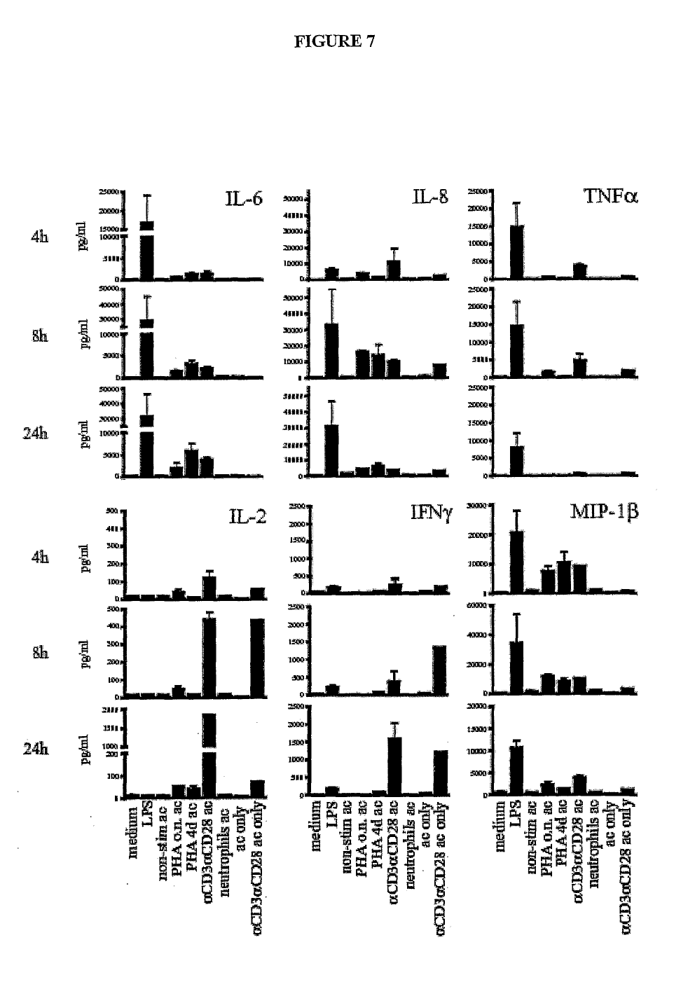

FIG. 7. Rapid cytokine release from DCs exposed to activated apoptotic T cells

Immature DCs were co-cultured with different apoptotic cells (ac) for 4 h, 8 h and 24 h and the culture supernatants were analyzed for presence of IL-6, IL-8, TNF-α, IL-2, IFN-γ and MIP-1β by Luminex technology. DCs cultured in only medium were negative control and LPS, which is a potent DC activator, was used as positive control. The apoptotic cells were from freshly isolated PBMCs (non-stim ac), PBMCs activated with PHA over night (PHA o.n. ac), PBMCs activated with PHA for 4 days (PHA 4d ac) or PBMCs activated with anti-CD3 and anti-CD28 mAb over night (αCD3αCD28 ac). Non-stimulated apoptotic PBMCs or anti-CD3/anti-CD28 activated apoptotic PBMCs alone without addition of DCs were also included as a control for cytoline release from the apoptotic cells per se.

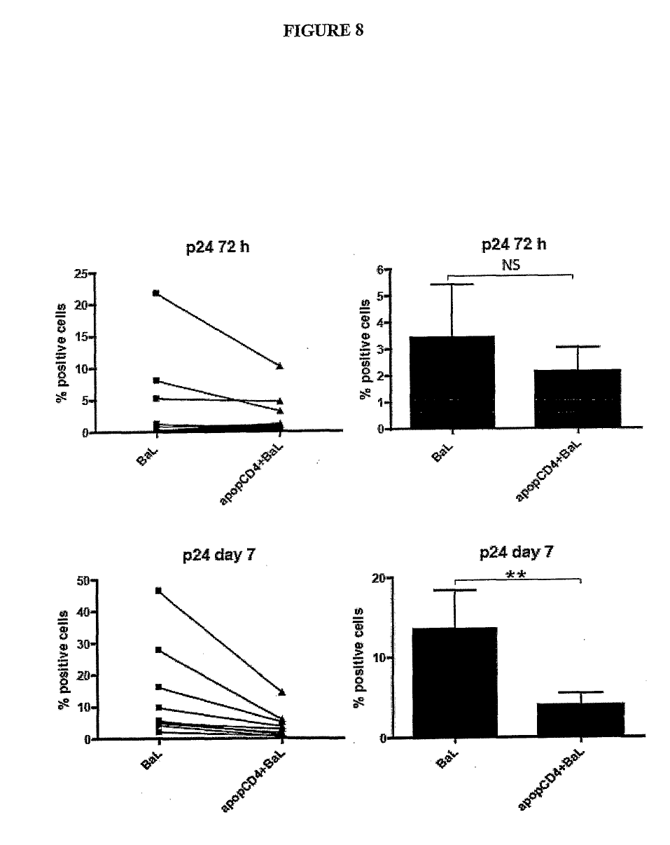

FIG. 8. Reduced frequency of HIV-1 infected DCs after co-culture with apoptotic activated T cells

Immature DCs were exposed to HIV-1_{BaL} (BaL) or HIV-1_{BaL} and apoptotic anti-CD3 and anti-CD28 activated CD4⁺ T cells (apopCD4+BaL). The frequency of infected DCs was assessed by flow cytometry of intracellular p24 staining after 72 hours and 7 days. The left panel shows individual results from nine donors and the right panel depicts the average frequency of p24 positive DCs±SD. Significant differences were assessed by non-parametric Wilcoxon test and are indicated by ** (P<0.01).

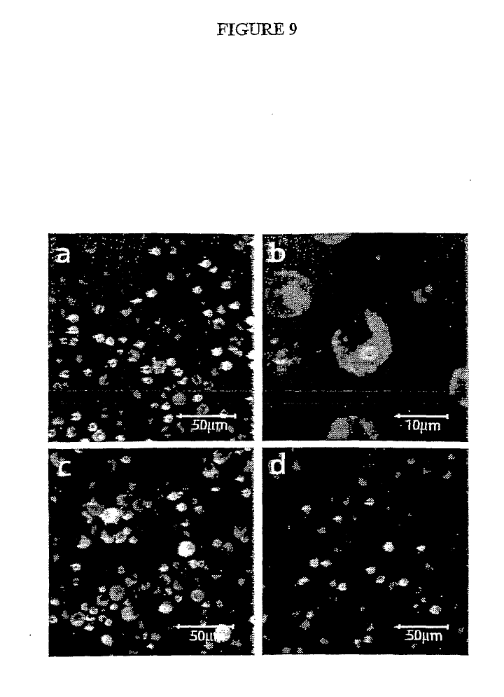

FIG. 9. Human monocyte derived DCs ingest apoptotic PBMCs

Immature monocyte derived DCs were labelled with PKH26 after 6 days of culture. PBMCs were labelled with PKH67 and thereafter induced to undergo apoptosis by γ-irradiation. Immunofluorescence images of DCs co-cultured with apoptotic PBMCs for 4 hours. DCs that have phagocytosed apoptotic cells (ac) give rise to a yellow appearance in the overlay picture (a). High magnification image reveals an apoptotic body within a DC after 4 hours of co-culture (b). After 24 hours of culture, the image reveals that a high frequency of the DCs have taken up ac (c). Cytochalasin D was added to the co-cultures in order to block phagocytic uptake of ac. Negative control was harvested after 24 hours of DC/ac co-culture (d).

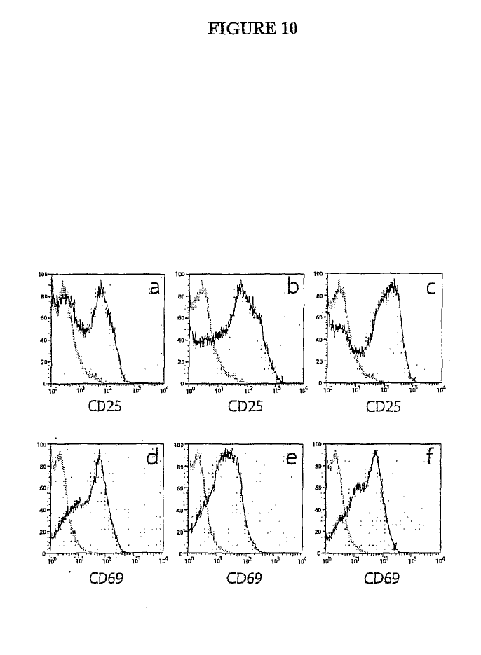

FIG. 10. Characterization of activated PBMCs

Human PBMCs were activated with PHA over night (a, d) or for 4 days (b, e) or were treated with αCD3 and αCD28 antibodies over night (c, f). Non-activated and activated PBMCs were stained for T-cell activation markers CD25 and CD69. Samples were analysed by flowcytometry and gates were set on lymphocytes. The stainings show up-regulation of CD25 and CD69 in antibody- and PHA stimulated PBMCs (black line) as compared to non-activated cells (grey line).

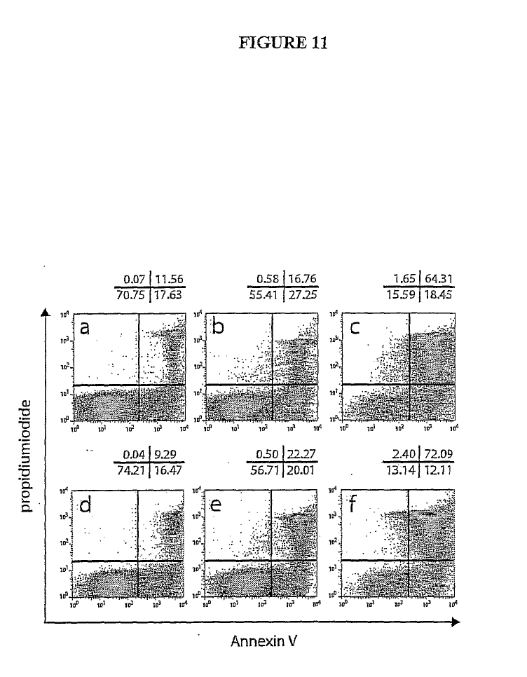

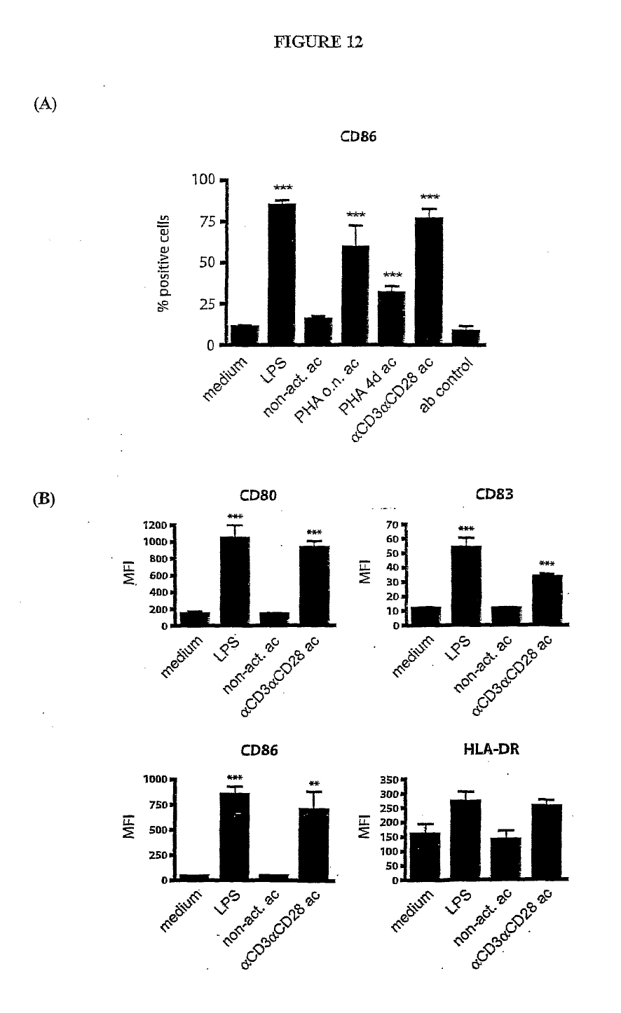

FIG. 11. Apoptosis induction in resting and activated PBMCs

Non-activated (a, b, c) and αCD3αCD28 activated (d, e, f) PBMCs were stained with annexin V and PI before gamma-irradiation (a, d) and 6 hours (b, e) or 24 hours (c, f) after irradiation to determine the frequency of apoptotic and necrotic cells in the populations. Samples were analysed by flow cytometry and the total PBMC population was included in the analysis. Both in resting and in activated cells an increased frequency of annexin V positive, apoptotic cells and annexin V-, PI double positive, necrotic cells were seen after gamma-irradiation.

FIG. 12. Activated, apoptotic PBMC induce maturation in human monocyte derived dendritic cells

DCs were co-cultured with apoptotic cells derived from non-activated PBMC (non-act. ac), PHA activated PBMC stimulated over night (PHA o.n. ac) or for 4 days (PHA 4d ac), anti-CD3/CD28 activated (aCD3aCD28 ac). Control samples included DCs cultured in medium or mAb (ab control). LPS was used as a positive control for induction of DC-maturation. DCs were co-cultured with ac for 72 h before flow cytometry analyses were performed. (a) depicts the frequency of CD86 positive cells and (b) the mean fluorescence intensity. n=16 for medium, LPS, DC, non-act ac, PHA 4d ac, n=11 for aCD3aCD28 ac, n=4 for PHA on ac and n=6 for ab control. In (b) n=6 for all samples. Significant up-regulation of co-stimulatory molecules as compared to medium control is indicated as *** (p≦0.0001).

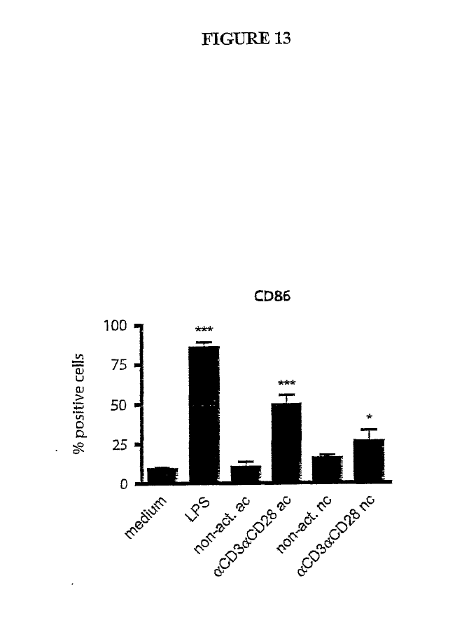

FIG. 13. Resting, necrotic PBMC are not able to induce DC maturation

DCs were co-cultured with apoptotic cells derived from non-activated PBMC (non-act. ac) (n=5), anti-CD3/CD28 activated (aCD3aCD28 ac) (n=5), or non-activated necrotic PBMCs (non-act nc) (n=22) and anti-CD3/CD28 activated necrotic PBMCs (aCD3aCD28 nc) (n=5). Control samples included DCs cultured in medium (n=8). LPS (n=8) was used as a positive control for induction of DC-maturation. DCs were co-cultured with ac for 72 h before flow cytometry analyses were performed. Gates were set on large, CD1a⁺ cells. Significant differences as compared to medium control are indicated as *(p≦0.05) or *** (p≦0.0001).

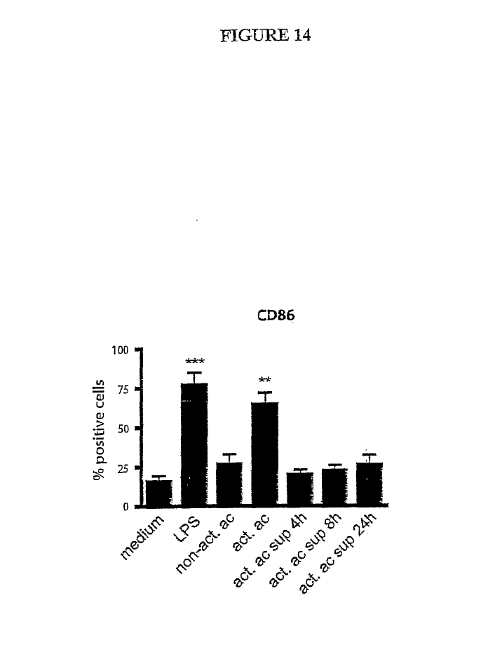

FIG. 14. Supernatants from apoptotic PBMCs do not have the capacity to induce DC maturation

Supernatants from αCD3αCD28 activated, irradiated PBMCs (act ac sup) were collected after 4, 8 and 24 hours. Supernatants were subsequently added to immature DC at day 6. Simultaneously, non-activated and αCD3αCD28 activated, irradiated PBMCs from the corresponding donors were added. Co-cultures were incubated for 72 hours. Cells were then stained for CD86 and analysed by flow cytometry. In the graph presented n=4 for medium control, LPS control, DC+non-activated apoptotic cells and DC+supernatant 24 hours, n=5 for DC+αCD3αCD28 activated apoptotic cells and n=6 for DC+supernatants 4 hours and 8 hours. Significant differences as compared to medium control are indicated as ** (p≦0.01) or *** p≦0.0001).

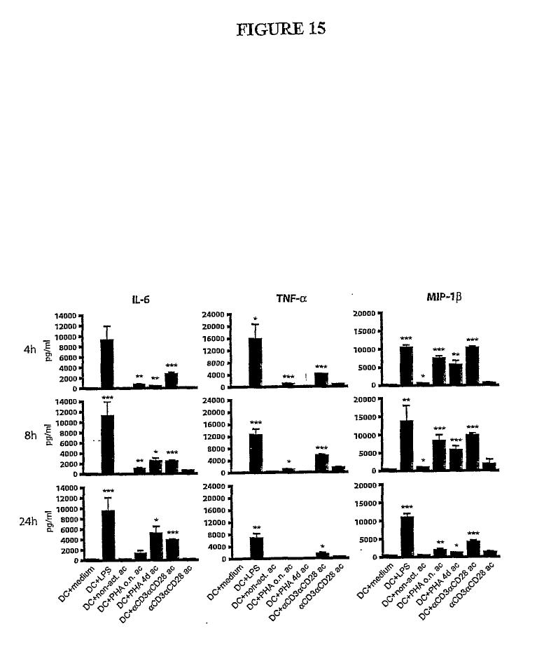

FIG. 15. Apoptotic PBMCs induce pro-inflammatory cytokine release in DC

Immature DCs were co-cultured with non-activated apoptotic cells (non-act. ac), apoptotic cells activated with PHA o.n (PHA o.n. ac). or for 4 days (PHA 4d) or αCD3αCD28 activated apoptotic cells (αCD3αCD28 ac). Supernatants from the co-cultures or from αCD3αCD28 ac alone were collected after 4, 8 and 24 hours of incubation. These were analysed for their contents of IL-6, TNFα, MIP-1β, IL-10 and IL-12p70 by Luminex. No production of IL-10 or IL-12 could be detected in any of the samples (not shown). For IL-6 supernatants n≧4 except for DC+apoptotic cells only where n=1. For TNFa supernatants n≧6 except for DC+apoptotic cells only where n=2. For MIP-1β n≧6 except for DC+apoptotic cells only where n=2. For statistical comparison of samples where n≧4 unpaired t tests were used and significant differences are indicated as *(p≦0.05), ** (p≦0.01) or *** (p≦0.0001).

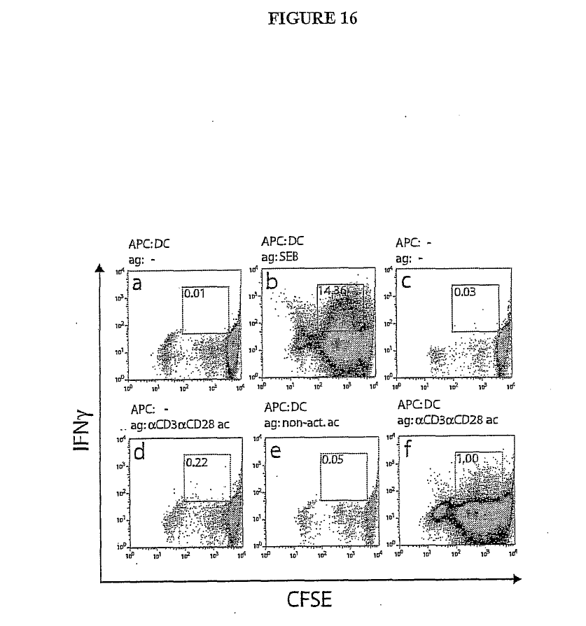

FIG. 16. Allo-antigen presentation and T-cell activation by DCs after uptake of activated, apoptotic PBMCs

Immature DCs were co-cultured with non-activated or activated allogeneic ac. In control wells medium only (a) or activated ac only (d) were added. After 48 h CFSE labelled autologous T-cells were added to all wells. SEB was added as a positive control (b). At day 3, 4, 5 or 6 after T-cell addition the cultures were stained for cell surface markers and intracellular IFNγ and were analysed by flowcytometry. Gates were set on CD3⁺, CD1a⁻ cells. In medium- and T-cells only controls (a, c) and in samples where DCs were given resting ac (e) or where autologous T-cells encountered ac only (d) no proliferation or IFNγ production was detected at any of the timepoints analysed. T-cell division and IFNγ production was detected at day 3 and peaked at day 4 in positive control (b) and in samples where DCs were co-cultured with activated ac (f). The figure shows cells collected from 1 representative donor out of 6 at day 4 of the experiment and numbers indicate percentages of IFNγ⁺ T-cells

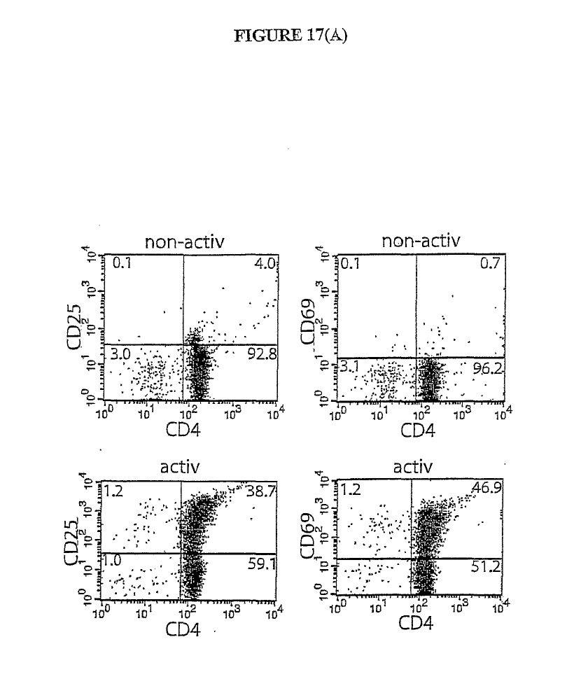

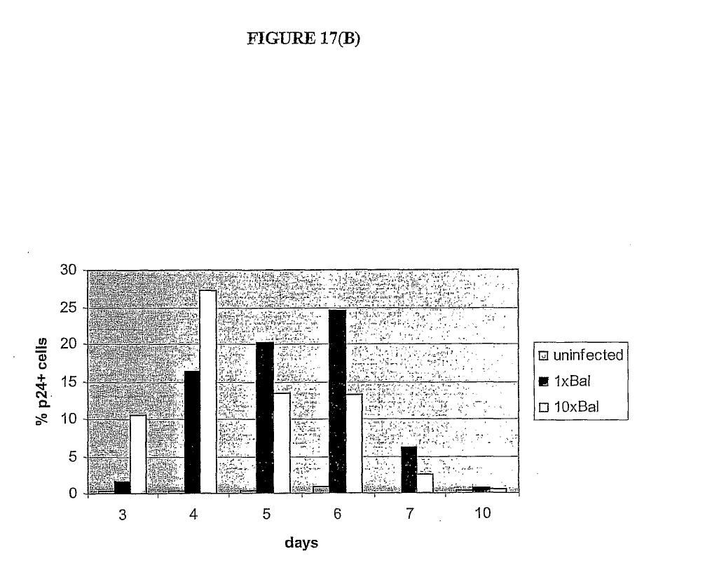

FIG. 17. Phenotypic characterization of apoptotic activated HIV-1 infected T cells

CD4⁺ T cells were isolated from healthy blood donors and put in culture without any additional stimulation (non-activ), or activated with anti-CD3 and anti-CD28 mAbs over night. The cells were either stained directly after the culture period or stained after a freezing period in DMSO. The recovered cells were stained with anti-CD4, anti-CD25 and anti-CD69 mAbs and analysed for surface expression by flow cytometry. Data are shown on gated on lymphocytes. The quadrants are set based on control stainings and the numbers depicts the frequency of cells in each quadrant (A). CD4⁺ T cells were activated with anti-CD3 and anti-CD28 mAb over night before they were infected with either 1xBaL stock or a 10xBaL stock. The frequency of infection was measured by intracellular p24 staining and quantified by flow cytometry. The kinetics of infection in CD4⁺ T cells of one representative experiment is shown (B).

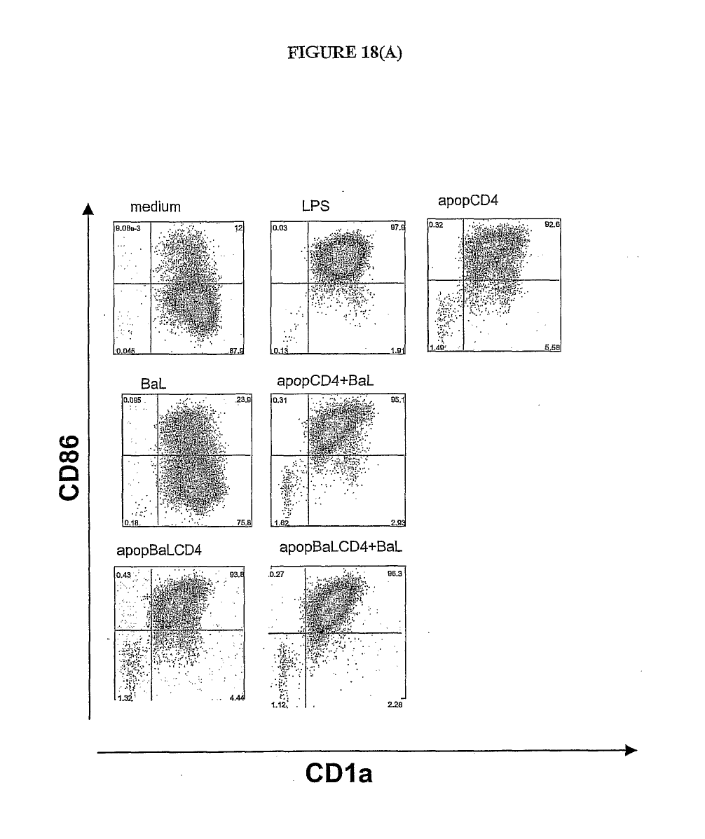

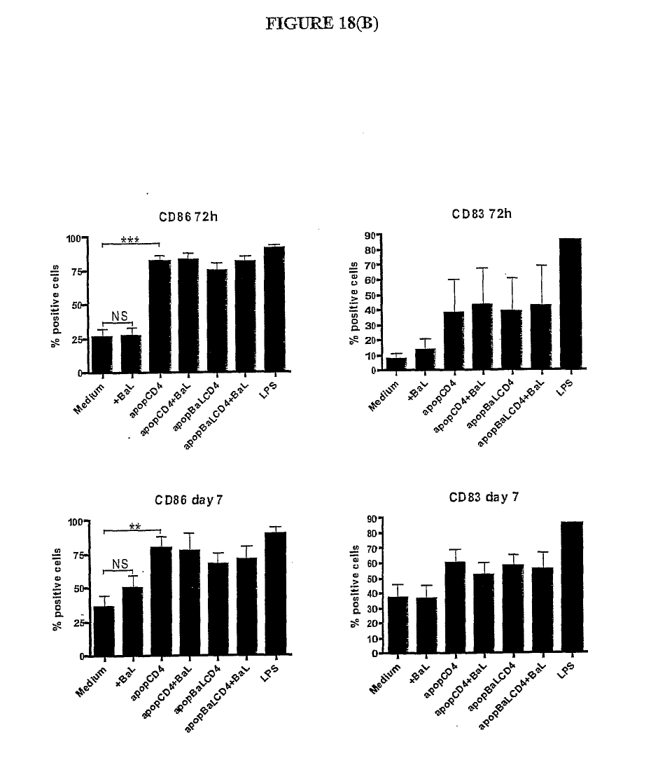

FIG. 18. Apoptotic activated HIV-1 infected T cells induce CD86 and CD83 expression in human DCs

Human in vitro differentiated monocytes cultured for 6 days in the presence of IL-4 and GM-CSF were used as source of human immature DCs as defined by their expression of CD1a, lack of CD14 and low expression of CD40, CD80, CD86 and CD83. These immature DCs were co-cultured with different apoptotic cells for 72 hours or 7 days and then analyzed for expression of CD86 molecules by flow cytometry. Gates were set on large CD1a⁺CD3⁻ cells. LPS, which is a potent DC activator, was used as positive control and DCs cultured in medium only was used as negative control.

Immature DCs were cultured in medium, in the presence of HIV-1_{BaL} (+BaL), apoptotic anti-CD3 and anti-CD28 activated CD4⁺ T cells (apopCD4), apoptotic anti-CD3 and anti-CD28 activated CD4⁺ T cells in the presence of free HIV-1_{BaL} (apopCD4+BaL), apoptotic activated HIV-1_{BaL} infected CD4⁺ T cells (apopBaLCD4), apoptotic activated HIV-1_{BaL} infected CD4⁺ T cells in the presence of free HIV-1_{BaL} (apopBaLCD4+BaL). Gates were set on large CD1a⁺CD3⁻ cells. Representative flow cytometry data after 7 days of co-culture are shown in (A). The average frequency of CD86 expressing DCs±SD of at least 11 donors at 72 h and seven donors at 7 days are depicted in (B). The CD83 expression on DCs before and after co-culture was examined in four donors. Significant differences were assessed by non-parametric Mann-Whitney test and are indicated by ** (P<0.01) and *** (P<0.001), respectively.

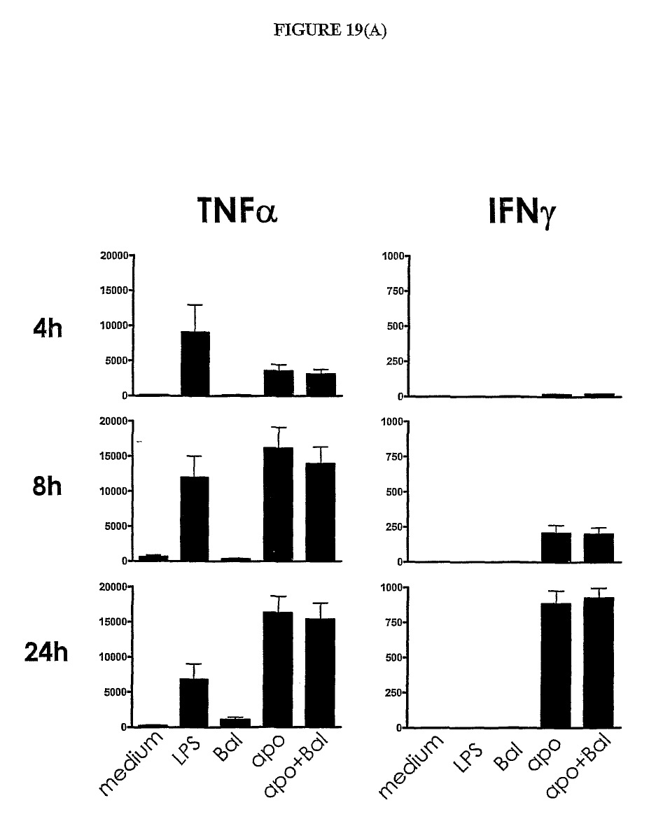

FIG. 19. Rapid cytokine release from DCs exposed to activated apoptotic CD4⁺ T cells

Immature DCs were co-cultured with different apoptotic cells for 4 h, 8 h and 24 h and the culture supernatants were analyzed for presence of IL-6, IL-8, TNF-α, IL-2, IFN-γ, MIP-1α and MIP-1β by Luminex. DCs cultured in only medium were negative control and LPS, which is a potent DC activator, was used as positive control. DCs were exposed to HIV_{BaL} (BaL), antiCD3 and anti-CD28 activated apoptotic CD4 T cells (apo) or antiCD3 and anti-CD28 activated apoptotic CD4 T cells in the presence of HIV_{BaL} (apo+Bal). The results shown are mean±SD from seven donors. The released TNF-α and IFN-γ, are shown in (A) and MIP-1α and MIP-1β in (B).

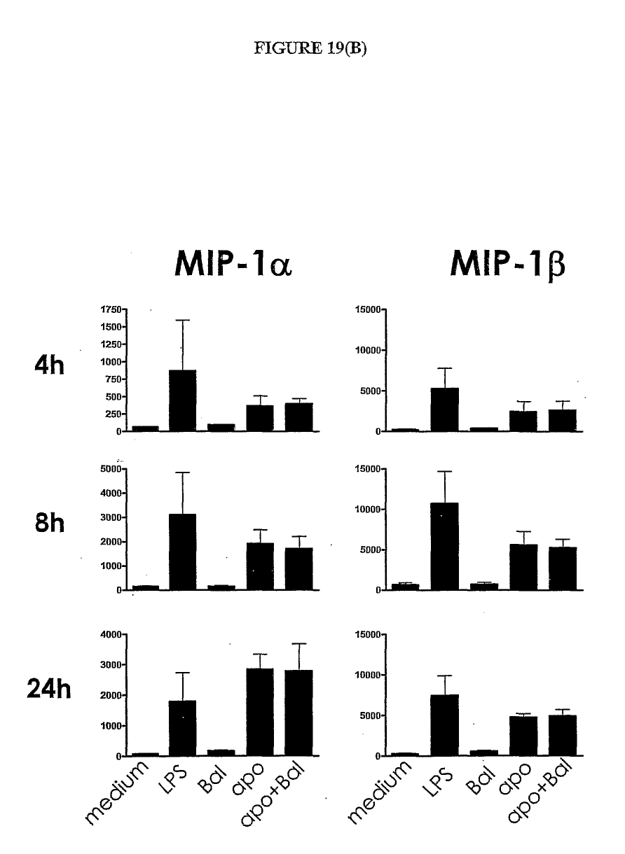

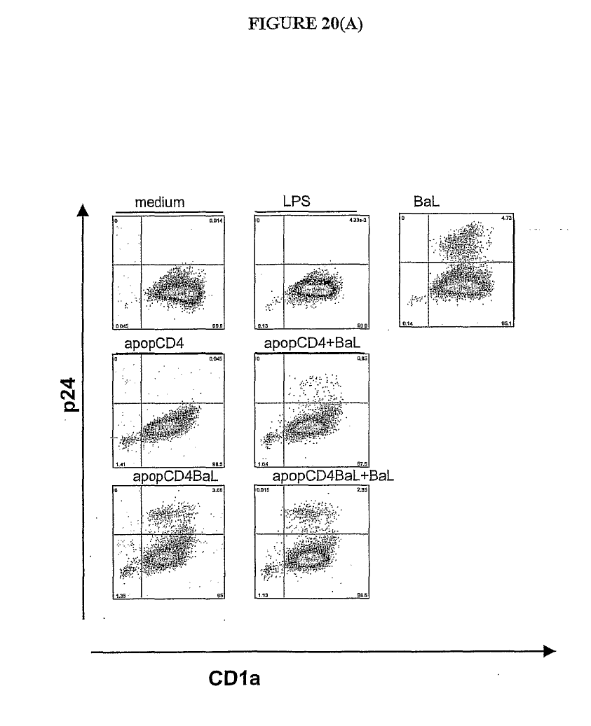

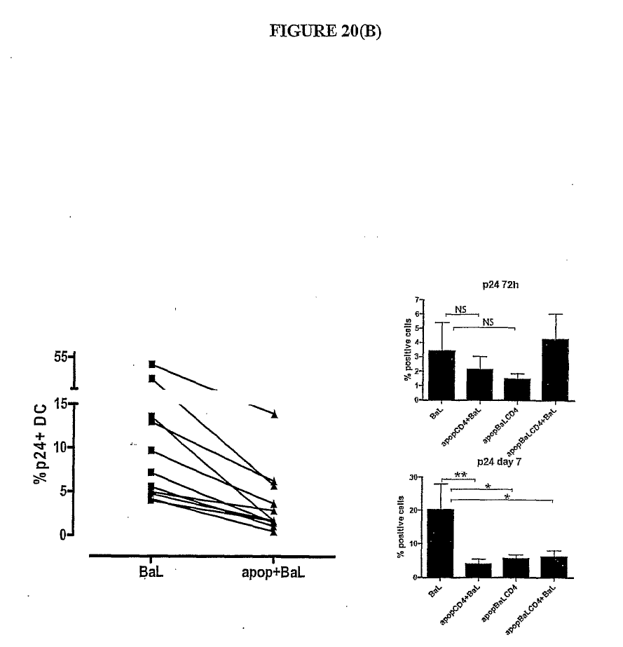

FIG. 20. Reduced frequency of HIV-1 infected DCs after co-culture with apoptotic activated T cells

Immature DCs were exposed to HIV-1_{BaL} (BaL), apoptotic anti-CD3 and anti-CD28 activated CD4⁺ T cells (apopCD4), apoptotic anti-CD3 and anti-CD28 activated CD4⁺ T cells in the presence of HIV-1_{BaL} (apopCD4+BaL), apoptotic anti-CD3 and anti-CD28 activated HIV-1_{BaL} infected CD4⁺ T cells (apopCD4BaL) or apoptotic anti-CD3 and anti-CD28 activated HIV-1 BaL infected CD4⁺ T cells in the presence of free virus (apopCD4BaL+BaL). The frequency of infected DCs was assessed by flow cytometry of intracellular p24 staining after 72 hours and 7 days. Panel A shows representative staining after 7 days of infection. (B) The left panel shows individual results from eleven donors and the right panel depicts the average frequency of p24 positive DCs±SD. Significant differences were assessed by non-parametric Wilcoxon test and are indicated by ** (P<0.01).

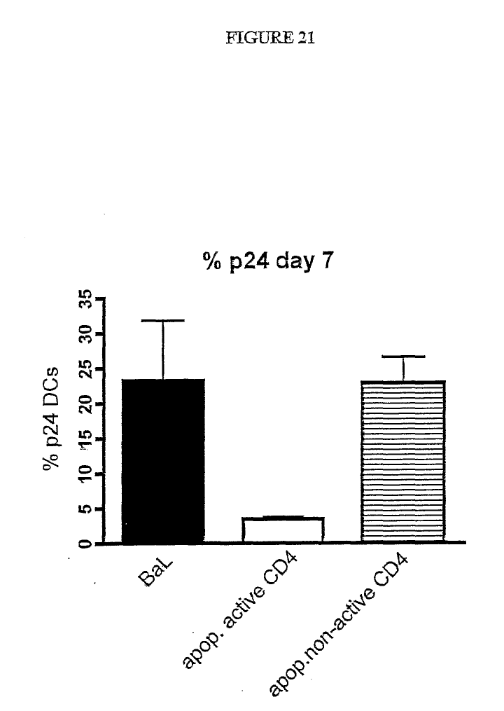

FIG. 21. Reduced frequency of HIV-1 infected DCs after co-culture with apoptotic activated but not apoptotic non-activated primary T cells

Immature DCs were exposed to HIV-1_{BaL} (BaL), apoptotic anti-CD3 and anti-CD28 activated CD4⁺ T cells (apop. activeCD4) or non-activated primary CD4+T cells (apop.non-active CD4) in the presence of HIV-1_{BaL}. The frequency of infected DCs was assessed by flow cytometry of intracellular p24 staining after 7 days. Results are shown as mean±SD p24 expressing DCs from at least three donors.

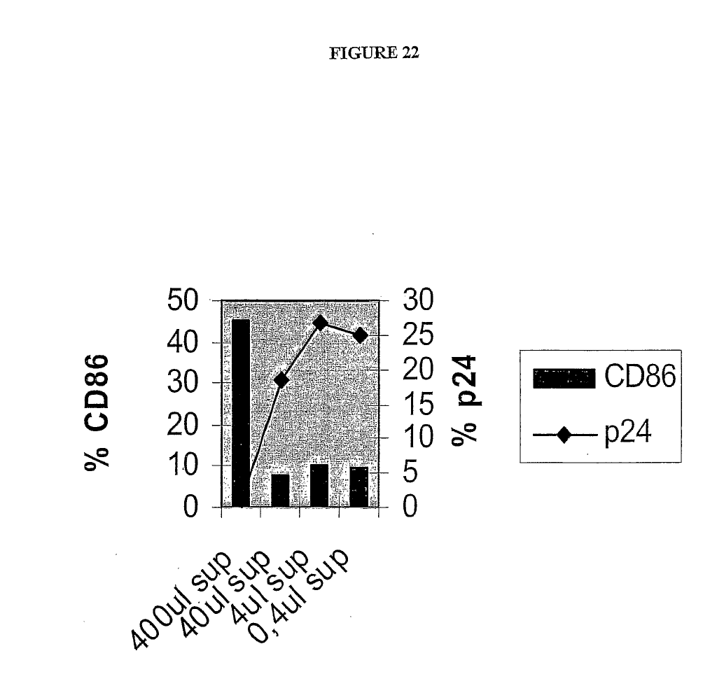

FIG. 22. Induction of maturation and reduced frequency of HIV-1 infected DCs after exposure to supernatant collected from co-cultures with DCs and apoptotic activated T cells

Supernatant were collected from co-cultures with DCs and apoptotic activated T cells after 24 hours. Immature DCs were exposed to the supernatants in increasing concentrations (final volume 1 ml) in the presence of HIV-1_{BaL}. The expression of CD86 and intracellular p24 expression were measured by flow cytometry after 72 hours and 7 days, respectively. One representative experiment out of two is shown.

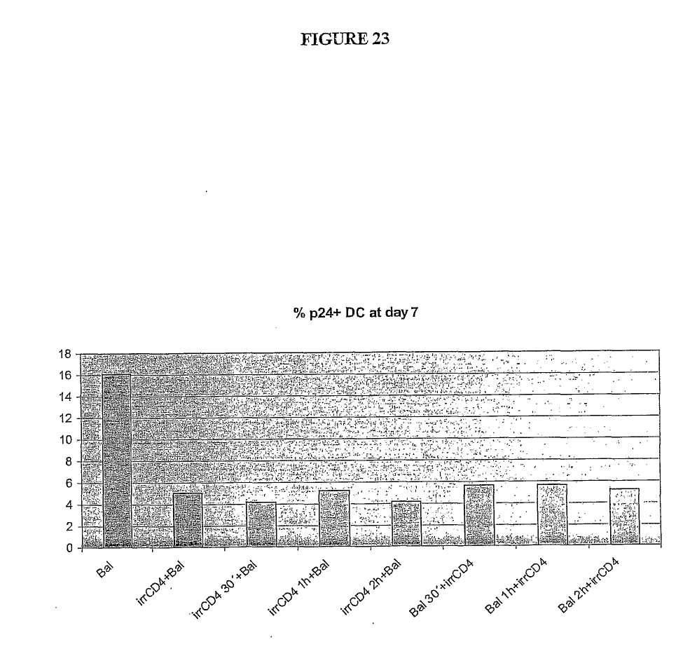

FIG. 23. Reduced frequency of HIV-1 infection in DCs after co-culture with apoptotic activated T cells both pre- and post-HIV-1_{BaL} exposure

Immature DCs were exposed to HIV-1_{BaL} (BaL) or both BaL and apoptotic anti-CD3 and anti-CD28 activated CD4⁺ T cells (irrCD4). The apoptotic activated CD4⁺ T cells were added at the same time as the virus (irrCD4+Bal), 30 min, 1 h or 2 h prior to addition of BaL or conversely, the DC cultures were first incubated with BaL for 30 min, 1 h or 2 h prior to addition of apoptotic activated CD4⁺ T cells. The frequency of infected DCs was assessed by flow cytometry of intracellular p24 staining after 7 days.

### EXAMPLES

### Example A

### Materials and Methods

### In Vitro Differentiation of Dendritic Cells

CD14⁺ monocytes were enriched from PBMCs from healthy blood donors by negative selection using RosetteSep Human Monocyte Enrichment (1 mL/10 mL blood; Stem Cell Technologies, Vancouver, BC, Canada). Monocytes were separated using lymphoprep (Nycomed, Oslo, Norway) density gradient. Cells were cultured for 6 days in medium (RPMI 1640 supplemented with 1% HEPES [N-2-hydroxyethylpiperazine-N′-2-ethanesulfonic acid], 2 mM L-glutamine, 1% Streptomycin and penicillin, 10% endotoxin-free foetal bovine serum (FBS); GIBCO Life Technologies, Paisley, United Kingdom) and recombinant human cytokines IL-4 (6.5 ng/mL; R&D Systems, Minneapolis, Minn.) and granulocyte macrophage-colony-stimulating factor (GM-CSF; 250 ng/mL; Peprotech, London, UK), to obtain immature dendritic cells.

### Activation of PBMC and T Cells

CD4⁺ and CD8⁺ T cells were enriched from healthy blood donor PBMCs by negative selection using RosetteSep's Human CD4⁺ or CD8⁺ T cell Enrichment (1 mL/10 mL blood respectively; Stem Cell Technologies). T cells and PBMCs were separated using lymphoprep density gradient (Nycomed, Oslo, Norway). Cells were frozen in FBS and 10% dimethylsulphoxide (DMSO) or were added to flasks containing 1% Sodiumpyruvate, monoclonal anti-human CD3 (2 μg/ml; clone OKT 3; Ortho Biotech Inc. Raritan, N.J.), that was adhered to the plastic during one hour in 4° C., and soluble monoclonal anti-human CD28 (2 μg/ml; L293; BD Biosciences, San Diego, Calif.). After stimulation cells were frozen in FBS/DMSO. PBMCs were also cultured over night or for 4 days in medium containing phytohemagglutinin (PHA; 2.5 ug/mL; SIGMA, St Louis, Mo.) and were then frozen in FBS/DMSO.

### HIV-1 Virus Growth and Preparation

The CCR5-uring HIV-1_{BaL} isolate (National Institutes of Health (NIH) AIDS Research and Reference Reagent Program, Division of AIDS, National Institute of Allergy and Infectious Diseases (NIAID), NIH) was grown on PBMC cultures stimulated with PHA (Sigma, St Louis, Mo.) and IL-2 (Chiron, Emeryville, Calif.). To concentrate the virus and to minimize the presence of bystander activation factors in the supernatant that could induce DC maturation, the virus was ultracentrifuged (138 000 g (45 000 rpm), 30 minutes, 4° C., Beckman L-80 Ultracentrifuge, rotor 70.1; Beckman Coulter, Fullerton, Calif.) and the virus pellet was resuspended in RPMI 10% FBS to obtain a 10× virus concentrate. The viral titer of the HIV-1_{BaL} stock was determined by p24 enzyme-linked immunosorbent assay (ELISA; Murez HIV antigen Mab; Abbott, Abbott Park, Ill.) according to manufacturer's protocol. Samples were analyzed in serial dilutions in duplicate. The 10×HIV-1_{BaL} stock had an HIV-1 p24 Gag content of 11.7 μg/mL. The HIV-1_{BaL} stock was also characterised by determining the level of active reverse transcriptase (RT; Lenti RT; Cavidi Tech, Uppsala, Sweden). The 10×HIV-1_{BaL} stock used contained 15 000 pg active RT/mL.

### HIV-1 Infection of T Cells and Dendritic Cells

CD4⁺ T cells were isolated from healthy blood donor PBMCs by negative selection using RosetteSep's Human CD4⁺ T cell Enrichment (1 mL/10 mL blood respectively; Stem Cell Technologies) and activated with anti-CD3 (2 μg/ml; clone OKT 3; Ortho Biotech Inc. Raritan, N.J.) and anti-CD28 mAb (2 μg/ml; L293; BD Biosciences, San Diego, Calif.) over night. The cells were then incubated with 10×HIV-1_{BaL} or 1×HIV-1_{BaL} stocks (200 μl HIV-1_{BaL} stock to 1×10⁶ CD4⁺ T cells) in the presence of IL-2 (Chiron, Emeryville, Calif.). The frequency of infected cells was analyzed by intracellular p24 staining day 3, 4, 5, 6, 7 and 10 after infection. The obtained infected cells were frozen in FBS/DMSO until use. A quantity of 200 μL of 1×HIV-1 BaL or mock was added to 5×10⁵ immature DCs/mL in a 24-well plate (Costar Corning, Corning, N.Y.) to a final volume of 1.0 mL per well. The frequency of infected DCs was determined by intracellular p24 staining after 72 hours and 7 days of infection.

### Transfection Using Nanoparticles

Nanotechnologies offer an attractive alternative method of transferring both DNA and proteins into target cells that could be used for vaccination purposes. However, if introduced to non-separated cell populations, e.g. bulk peripheral blood cells, nanoparticles are taken up by many different cell types resulting in a low transfer efficiency into antigen presenting cells. Immunisation in vivo with nanoparticles can also lead to dilution of the particles due to uptake of nanoparticles into non-antigen presenting cells. Moreover, nanoparticles do not have any known intrinsic adjuvant effects.

A solution to these problems is to combine the use of nanoparticles as carriers of antigen with the apoptotic cell technology of the present invention, which targets antigen into phagocytic antigen presenting cells and in addition provides adjuvant signal(s). One embodiment involves loading HIV-DNA and/or HIV-protein conjugated nanoparticles into selected T-cell subsets (e.g. activated CD4+ T cells) in vitro and thereafter apoptosis is induced by for example gamma-irradiation. The HIV-DNA/protein nanoparticle loaded apoptotic activated T cells are used as immunogen to allow for induction of primary immune responses.

Exemplary protocol: Iron oxide nanoparticles (Ferridex IV) are obtained from for example Berlex Laboratories, Wayne N.J. To facilitate cellular uptake the negatively charged iron oxide particles will be conjugated to protamine sulphate. Both ferumoxide nanoparticles and protamine sulphate are FDA approved agents, thereby facilitating translation to human therapy protocols. For vaccination purposes the nanoparticles are either conjugated to DNA or proteins. The nanoparticles are incubated with live T cells to allow uptake. The T cells can be activated either before or after uptake of nanoparticles. Activation can be performed by using for example anti-CD3 and anti-CD28 mAbs. The uptake of nanoparticles is assessed by microscopy and flow cytometry. The activated T cells are thereafter induced to undergo apoptosis and are used as immunogen. The nanoparticle-carrying apoptotic activated T cells can be immunized directly and uptake in antigen-presenting cells will occur in vivo. Alternatively, an additional step of co-culture with antigen-presenting cells such as dendritic cells can be performed in vitro before immunisation to the patient.

### Quantification of HIV-1 Protein in T Cells and Dendritic Cells

The frequency of HIV-1_{BaL} infection in DCs and T cells was determined by intracellular staining for the HIV-1 Gag protein p24. Cells were first stained for cell surface markers, then washed in PBS and fixed in 2% formaldehyde (Sigma) for 10 minutes at room temperature. Cells were washed in PBS with 2% FBS followed by a wash in PBS with 2% FBS, 2% HEPES and 0.1% Saponin (Sigma) to allow permeabilization of the cell surface membrane. Cells were incubated for 1-2 hour at 4° C. with the anti-p24 specific mAb (clone KC57; Coulter, Hialeah, FL) or the corresponding isotype control. Cells were washed in saponin solution to remove excessive antibody and resuspended in PBS. Expression was assessed by a FACSCalibur flow cytometer (Becton Dickinson).

### Generation of Apoptotic Cells and Apoptotic Cell Supernatants

Frozen T cells and PBMCs were thawed and washed 3 times in RPMI. Cells were induced to undergo apoptosis by γ-irradiation (150 Gy). The γ-irradiation induced apoptotic process has previously been demonstrated by morphological changes, flow cytometry and DNA fragmentation on agarose gels (Holmgren et al., 1999, Blood 93:3956; Spetz et al., 1999, J Immunol 163:736). Apoptosis was here confirmed by flow cytometry stainings with AnnexinV (Boehringer Mannheim, Mannheim, Germany) and propidium iodide (PI) (0.1 μg/sample; Sigma, Stockholm, Sweden) according to manufacturer's protocol. Supernatants were collected from live and irradiated cells after 4, 8 and 24 hours and were centrifuged at 1.4×10⁴ rpm for 30 min to remove possible cell debris.

### DC Co-Cultures

On day 6, immature DCs were counted and plated in 24-well plates, 5×10⁵ cells in 0.5 mL medium (RPMI supplemented with 10% FBS and recombinant human IL-4 and GM-CSP). Live or irradiated PBMCs or T cells were added to DCs in proportion 2:1 to a total volume of 1 mL. Supernatant (0.5 mL) from 10⁶ live/irradiated PBMCs and T cells, collected at 4, 8 and 24 hours, was also added to immature DCs. Supernatant was collected from co-cultures at 4, 8 and 24 hours. At 72 hours or after 7 days all samples were collected and DCs were characterized by flow cytometric analysis. Lipopolysaccharide (LPS 100 ng/mL, Sigma) was added as a positive control for activation/maturation of DCs.

### Phenotypic Characterization of DC, PBMC and T Cells

DCs were washed and resuspended in PBS with 2% PBS. They were incubated for 30 min in 4° C. with the following anti-human monoclonal antibodies (mAbs): CD1a (clone NA1/34, DAKO, Glostrup, Denmark), CD14 (clone TÜK4; DAKO), CD19 (clone HD37, DAKO), CD3 (clone SK7), CD83 (clone HB15e) and CD86 (clone 2331/FUN-1; all from BD Biosciences, San Diego, Calif.). PBMC and T cells were washed and incubated with anti-human monoclonal antibodies CD19 (clone HD37; DAKO), CD14 (DAKO), CD3 (clone SK7), CD4 (clone RPA-T4)+Streptavidin, CD8 (clone SK-1), CD154 (clone TRAP-1), CD25 (clone 2A3) and CD69 (FN50; all from BD). Cell surface expression was measured by a FACScalibur flow cytometer (Becton Dickinson) and at least 10⁵ cells/sample were collected. Co-culture samples were at 72 hours or 7 days washed and incubated with the previously mentioned CD1a, CD4, CD8, CD83 and CD86. DCs were also stained with Annexin V as in preceding paragraph to detect possible apoptotic DCs.

### Cytokine/Chemokine Production

Supernatants from PBMC, T cells and co-cultures were analysed for cytokine/chemokine content by using a Bio-Plex assay (Biosource, Nivelles, Belgium). The assay was used according to manufacturer's protocol and a Luminex reader (Luminex Corporation, Austin Tex., USA) was used to simultaneously quantify the concentration of IL-6, IL-8, IL-2, IL-10, IL-12, TNFα, IFNγ, MIP-1α and MIP-1β in the supernatants.

### Results

To investigate whether activated apoptotic T cells have the capacity to provide any activation/maturation signal to dendritic cells, we induced apoptosis in PBMCs or CD3⁺ T cells activated with either PHA or anti-CD3 and anti-CD28 mAbs and thereafter added them to human in vitro differentiated dendritic cells. The efficiency of T cell activation was determined by analyzing induction of CD25 and CD69 expression on T cells (FIG. 2A). We detected increased expression of both CD25 and CD69 molecules on CD4⁺ T cells after activation with either anti-CD3/CD28 mAbs or PHA. The frequency and level of CD25 and CD69 expression was similar after anti-CD3/CD28 mAbs and PHA stimulation. These findings suggest that the T cells were efficiently activated in the culture system used. The obtained PBMC or T cell preparations were thereafter frozen in DMSO until use. The day of experiment, the frozen cells were thaw, washed and induced to undergo apoptosis by gamma-irradiation. Apoptosis induction was measured by performing Annexin-V and PI staining, which were quantified by flow cytometry (FIG. 2B). Early apoptotic cells are defined as Annexin-V⁺ and PI⁻. Later during apoptosis the cell membrane is permeabilized allowing uptake of PI. However, the membrane in freshly isolated cells sometimes exposes phosphatidyserine residues that bind Annexin-V, therefore also freshly isolated cells contain a proportion of Annexin-V positive cells. We found that frozen cells displays a higher proportion of Annexin-V⁺ cells compared to the freshly isolated cells. The newly thaw cells were exposed to gamma-irradiation at room temperature to induce apoptosis. We could not detect any increased binding of Annexin-V just after exposure to gamma-irradiation. The subsequent progression of apoptosis (Annexin-V⁺/PI⁻) and secondary necrosis defined as Annexin-V⁺/PI⁺ requires further incubation in 37° C. FIG. 2B depicts the characteristic phenotype of the cells when used as an antigen delivery system.

To investigate whether apoptotic T cells may per se be able to provide any adjuvant activity we used in vitro differentiated monocytes cultured for 6 days in the presence of IL-4 and GM-CSF as source of human immature dendritic cells as defined by their expression of CD1a, lack of CD14 and low expression of CD40, CD80, CD86 and CD83. These immature dendritic cells were co-cultured with apoptotic cells (ac) for 72 hours and then analyzed for expression of the co-stimulatory molecule CD86 (FIG. 3). Representative flow cytometric analyses are depicted in FIG. 3A and a summary of at least 8 experiments are shown in FIG. 3 B. The frequency of CD86⁺ DCs was 92.0±7.4% after LPS stimulation and the background medium control was 12.3±5.4%. A modest but significant increase in CD86 expression was detected after co-culture with non-activated PBMC (18.7±5.4%) as compared to the medium control. However, there was a more impressive induction of CD86 expression after co-culture with apoptotic PBMCs activated with PHA over night (48.4±23.0%). The kinetics of activation appeared to be of importance because PBMCs activated with PHA for 4 days were less efficient in inducing CD86 expression as compared with PBMCs activated with PHA over night (27.8±19.5%). To investigate whether other T cell activators could be used in order to induce the adjuvant properties in T cells, we stimulated PBMCs with anti-CD3 and anti-CD28 mAbs over night before apoptosis induction. We detected a robust induction of CD86 after co-culture with apoptotic anti-CD3/CD28 stimulated PBMCs (87.5+7.3%), which were comparable to CD86 expression induced by LPS. Altogether, these findings suggest that antiCD3/CD28 stimulation of T cells prior to apoptosis induction is an efficient way of inducing adjuvant properties in apoptotic cells.

To directly address whether apoptotic CD4⁺ and/or CD8⁺ positive T cells could provide the activation/maturation signal to the DCs, we purified CD4⁺ or CD8⁺ T cells prior to activation with anti-CD3 and anti-CD28 mAbs. The frequency of DCs expressing CD86 molecules after co-culture with live non-activated CD4⁺ or CD8⁺ T cells were 18.6% and 6.7%, respectively (FIG. 4). There was a significant increase in CD86 expression after co-culture with live activated CD4⁺ but not activated CD8⁺ T cells as compared to CD86 expression induced after co-culture with the non-activated T cell populations. Similarly, co-culture with apoptotic non-activated CD4⁺ or CD8⁺ T cells did not induce any up regulation of CD86 molecules, while apoptotic antiCD3/CD28 activated CD4⁺ T cells were able to provide a signal that resulted in efficient up regulation of CD86. There was a tendency, but it did not reach significance, that live and apoptotic activated CD8⁺ T cells could induce CD86 expression in DCs. Activated or non-activated necrotic primary CD4⁺ T cells were unable to deliver the activation/maturation signal in the co-culture system used here. We conclude from these experiments that activated live and apoptotic CD4⁺ T cells are efficient inducers of CD86 expression in DCs.

To investigate whether apoptotic activated CD4⁺ T cells infected with HIV-1 could provide the activation/maturation signal to DCs, we infected activated (anti-CD3 and anti-CD28 stimulated) CD4⁺ T cells with HIV-1 and induced apoptosis by exposure to gamma-irradiation. The kinetics of infection and a representative example of infection efficiency as determined by intracellular p24 staining is shown in FIG. 5. Batches of cells containing 20-40% HIV-1 infected cells, as measured by intracellular p24 staining, were frozen and subsequently used to prepare apoptotic HIV-1 infected cells.

DCs exposed to HIV-1_{BaL} were not induced to express CD86 either at 72 hours or after 7 days of culture (FIG. 6). However, co-culture with apoptotic activated HIV-1_{BaL} infected T cells resulted in induction of CD86. The activation/maturation signal provided by the activated CD4⁺ T cells occurred even in the presence of free HIV-1_{BaL}. We also determined whether induction of CD83, which is another molecule associated with DC maturation, was induced in the co-cultures. We could not detect induction of CD83 after exposure to HIV-1_{BaL}, while there was induction of CD83 after co-culture with activated CD4⁺ T cells. The pattern of expression was similar to CD86 expression and CD83 was induced after co-culture with activated CD4⁺ T cells even in the presence of HIV-1. These findings show that apoptotic activated CD4⁺ T cells is able to provide an activation/maturation signal to immature DCs even in the presence of HIV-1. Furthermore, a population of apoptotic activated T cells containing a high frequency of HIV-1 infected cells is also able to provide an activation/maturation signal to DCs. Altogether, these findings suggest that apoptotic activated T cells carrying HIV-1 may be used as an antigen transfer system that is able to induce certain DC activation/maturation.

To address whether cytokine production was induced in DCs after uptake of apoptotic activated T cells, we collected supernatants from the co-cultures after 4, 8 and 24 hours (FIG. 7). The Luminex technology, which allows simultaneous analyses of up to eight cytokines, was used. The secretion of IL-6 was detected as early as 4 hours of co-culture with activated T cells, but peaked at 24 hours. Both PHA and anti-CD3 and CD28 activated apoptotic cells could provide a signal that enabled IL-6 secretion. The IL-8 secretion peaked at 8 hours but was more difficult to delineate due to background secretion from the apoptotic cells per se. There was a rapid induction of TNF-α, primarily from the co-cultures with anti-CD3 and anti-CD28 activated cells. We could detect IL-2 and IFN-γ in the cultures but intracellular staining of these cytokines in dendritic cells has to be performed to determine whether this staining is due to secretion from the dendritic cells or whether it is only release from the apoptotic T cells. We could detect a rapid induction of MIB-1β in the cultures with activated apoptotic T cells and there was no background secretion from the apoptotic cells per se. Co-culture with non-stimulated T cells or neutrophils did not result in any secretion of mentioned cytokines. We could not detect any production of IL-10 or IL-12p70 regardless of which apoptotic cells were used. Only stimulation with CD40L provided strong induction of IL-10 and IL-12p70 (data not shown). Altogether, these findings suggest that activated apoptotic T cells are able to induce pro-inflammatory cytokine production in DCs but do not per se induce secretion of IL-12p70. Hence, the apoptotic activated T cell is able to induce DC activation/maturation to a certain point but additional signal is required to obtain IL-12p70 production. A similar profile of cytokine induction was also observed using apoptotic HIV-1 infected cells (data not shown). Hence, the HIV-1 infection in the apoptotic cells does not alter the cytokine expression profile of analysed cytokines in DCs.

The finding that several cytokines were released into the supernatants, including those with anti-HIV-1 activity, prompted us to ask the question whether co-culture with apoptotic activated CD4⁺ T cells could influence the efficiency of virus infection in DCs. We measured the rate of HIV-1 infection by determining the frequency of cells expressing intracellular p24 antigen as previously described (Smed-Sorensen et al., 2004, Blood 104:2810-7). Addition of 3′-azido-3′deoxythymidine (AZT) to the cultures inhibits detection of p24, suggesting that detected intracellular p24 in DCs is due to productive infection in DCs and not the result of uptake of viral particles or p 24 protein. In addition, the levels of HIV-1 p24 released in supernatants increase over time in the DC cultures exposed to HIV-1, as measured by ELISA (Smed-Sorensen et al., 2004, Blood 104:2810-7).

Immature DCs were exposed to HIV-1_{BaL} and we found a large donor variability regarding HIV-1 infection efficiency ranging from 0.1-21.7% after 72 hours incubation and between 2.1-46.4% after 7 days. We could not detect any significant reduction in intracellular p24 expression in the DCs co-cultured with apoptotic activated CD4⁺ T cells after 72 hours. However, after 7 days of culture all nine donors analyzed had a reduced frequency of p24+DCs in the cultures containing apoptotic CD4⁺ T cells as compared to DCs exposed only to HIV-1 BaL. These finding suggest that co-culture of DCs and apoptotic anti-CD3 and anti-CD28 activated CD4⁺ T cells results in an environment able to limit HIV-1 infection in DCs.

In summary, it was found that apoptotic activated HIV-1 infected CD4⁺ T cells are able to provide a maturation/activation signal to DC even in the presence of free HIV-1 virus. In addition, it was shown that simultaneous co-culture with apoptotic activated T cells leads to inhibition of virus replication in DCs. Altogether, these surprising findings demonstrate that apoptotic activated CD4⁺ T cells can be used as a vehicle for antigen delivery capable of providing an activation/maturation signal to antigen presenting cells.

### Example B

### Introduction

Dendritic cells (DCs) are potent antigen presenting cells that may have the capacity to stimulate naïve T helper cells and initiate primary T cell responses. DCs residing in peripheral tissues survey the microenvironment by engulfing both microbial material and dying cells of the host. The result of antigen presentation by DCs depends upon their activation/maturation status. Immature DCs require activation/maturation signals in order to undergo phenotypic and functional changes to acquire a fully competent antigen-presenting capacity. Activation/maturation of DCs involves several steps such as a transient increased capacity to take up antigen, migration towards draining lymph-nodes and simultaneous up-regulation of molecules including chemokine receptors and co-stimulatory molecules. Upon challenge with microbial or inflammatory stimuli DCs gain the ability to stimulate lymph-node-based naïve T helper (Th) cells and initiate primary T cell responses (1). Mature DCs in the lymph node provide Th cells with an antigen specific signal via MHC and a co-stimulatory signal via molecules such as CD80 and CD86 (2) (3). Th type 1 (Th1) cell priming is dependent on IL-12 production by DCs, initiated via CD40-CD40L interactions. Emerging data also supports the involvement of an additional signal contributing to the polarization towards Th1 or Th2 responses (4) (5) (6).

DC activation/maturation can be induced by a variety of signals. Among the most efficient are products of microbial origin termed pathogen-associated molecular patterns (PAMPs)(7). These are recognized by pattern-recognition receptors (PRRs), including members of the Toll-like receptor (TLR) family (8) (9). Ligation of these receptors leads to production of pro-inflammatory cytokines by DCs, such as type I interferons (IFNs), tumor necrosis factor (TNF) and interleukin 1 (IL-1), which also have been shown to influence DC activation ((10) (11) (12) (13) (14) (15) (6). Some mature DC features may therefore be due to secondary effects mediated by their own cytokine production. However, one report suggests that the inflammatory mediators released after TLR signalling are insufficient to induce full DC activation (6). DCs activated indirectly by inflammatory mediators were able to upregulate MHC molecules and co-stimulatory molecules and to drive T cell proliferation and clonal expansion, but lacked the ability to produce IL-12 p40, which correlates with an inability to promote Th1 effector differentiation (6). In addition, Blander and Medzhitov recently showed that the efficiency of MHC class II molecules antigen presentation on DCs depends on the presence of TLR ligands within phagocytosed cargo (16). Taken together, these data indicate that DCs are likely to be alerted by inflammatory mediators but will require PAMP recognition to develop into a fully mature DC with capacity to prime Th1 or Th2 cells.

These findings are in line with the hypothesis that “the immune system evolved to discriminate infectious non-self from non-infectious self” (17). This does however not serve as an explanation for responses generated in autoimmune disease, against tumours, against transplants or to viruses exploiting the host machinery for synthesis and thus may lack PAMPs. A different approach is engaged in the danger hypothesis (18) where it is suggested that dangerous antigens are discriminated from non-dangerous ones. Here, injured cells of the host function as endogenous adjuvants, giving rise to a danger signal that leads to activation of APCs and further stimulation of T-cells. In this hypothesis it is also argued that apoptotic cell-death is a frequent event during non-pathological conditions, why apoptotic cells alone would lack the capacity to signal danger. In contrast, necrotic cells generated under pathological circumstances are able to provide the danger signal capable of inducing an immune reaction.

Uric acid or heat shock proteins (HSPs) are released upon cell-death and have been suggested to function as endogenous adjuvants (19). In support of the danger theory are in vitro data showing that apoptotic cells are unable to induce maturation in DCs (10) (20) (21) (22). Apoptotic cells have also been reported to induce production of anti-, rather than pro-inflammatory cytokines in DCs ((23) (24) (25) (26) and there are in vivo data demonstrating tolerance induction by apoptotic cells (27) (28). Yet other studies have conversely shown immuno-stimulatory effects mediated by apoptotic cells (29) (30) (31) (32) (33) (34). Why apoptotic cells exhibit such diverse effects in the different studies is not clear.

We previously demonstrated that immunization with apoptotic HIV/Murine Leukaemia Virus infected cells could induce HIV-1 specific, both cellular and humoral, immune responses in vivo ((35). In this system, the infected cells were activated before apoptosis induction and administration. The responses elicited in vivo led us to investigate whether the activation state of the apoptotic cells was of importance in achieving the adjuvant effect. In the present example we have set up an in vitro system comparing the potential of resting, versus activated peripheral blood mononuclear cells (PBMCs) in providing human immature DCs with an activation/maturation signal. We show that activated cells, induced to undergo apoptosis by γ-irradiation, but not resting apoptotic cells, induce expression of co-stimulatory molecules and release of pro-inflammatory cytokines in DCs. Furthermore, we show that uptake of allogeneic, activated, apoptotic cells by DCs rendered them able to induce proliferation and IFNγ production in autologous T-cells. These findings demonstrate that primary, activated apoptotic cells are able to promote maturation of DCs and function as endogenous adjuvants in induction of specific T-cell responses.

### Material and Methods

### In Vitro Differentiation of Dendritic Cells

CD14⁺ monocytes were enriched from blood from healthy blood donors by negative selection using RosetteSep Human Monocyte Enrichment (1 mL/10 mL blood; Stem Cell Technologies, Vancouver, BC, Canada). Monocytes were separated using lymphoprep (Nycomed, Oslo, Norway) density gradient. Cells were cultured for 6 days in medium (RPMI 1640 supplemented with 1% HEPES [N-2-hydroxyethylpiperazine-N′-2-ethanesulforic acid], 2 mM L-glutamine, 1% Streptomycin and penicillin, 10% endotoxin-free foetal bovine serum (FBS); GIBCO Life Technologies, Paisley, United Kingdom) and recombinant human cytokines IL-4 (6.5 ng/mL; R&D Systems, Minneapolis, Minn.) and granulocyte macrophage-colony-stimulating factor (GM-CSF; 250 ng/mL; Peprotech, London, UK), to obtain immature dendritic cells.

### Activation of PBMCs

PBMCs were separated from healthy blood donors using lymphoprep density gradient (Nycomed, Oslo, Norway). CD4⁺ T cells were enriched by negative selection using RosetteSep's Human CD4⁺ T cell Enrichment (1 mL/10 mL blood respectively; Stem Cell Technologies). Cells were frozen in FBS and 10% dimethylsulphoxide (DMSO) or were directly cultured in RPMI containing 1% Sodiumpyruvate. Cells (10⁶/ml) were activated with phytohemagglutinin (PHA; 2.5 μg/mL; SIGMA, St Louis, Mo.) over night or for 4 days before they were frozen in FBS/DMSO. The monoclonal anti-human CD3 (2 μg/ml; clone OKT 3; Ortho Biotech Inc. Raritan, N.J.), was adhered to plastic during one hour in 4° C. before addition of soluble monoclonal anti-human CD28 (2 μg/ml; L293; BD Biosciences, San Diego, Calif.) and cells. After over night stimulation cells were frozen in FBS/DMSO.

### Generation of Apoptotic Cells and Apoptotic Cell Supernatants

Frozen PBMCs were thawed and washed three times in RPMI. Cells were induced to undergo apoptosis by γ-irradiation (150 Gy). The γ-irradiation induced apoptotic process has previously been demonstrated by morphological changes, flow cytometry and DNA fragmentation on agarose gels (36) (37). Apoptosis was here confirmed by flow cytometry stainings with AnnexinV (Boebringer Mannheim, Mannheim, Germany) and propidium iodide (PI) (0.1 μg/sample; Sigma, Stockholm, Sweden) according to manufacturer's protocol. Supernatants were collected from irradiated cells after 4, 8 and 24 hours and were centrifuged at 1.4×10⁴ rpm for 30 min to remove possible cell debris.

DC/Apoptotic PBMC co-Cultures

On day 6, immature DCs were counted and plated in 24-well plates, 5×10⁵ cells in 0.5 mL medium (RP supplemented with 10% FBS and recombinant human IL-4 and GM-CSF). Irradiated PBMCs were added to DCs in proportion 2:1 to a total volume of 1 mL. Supernatant (0.5 mL) from 10⁶ irradiated PBMCs, collected at 4, 8 and 24 hours, was also added to immature DCs. Supernatant was collected from co-cultures at 4, 8 and 24 hours. At 72 hours all samples were collected and DCs were characterized by flow cytometric analysis. Lipopolysaccharide (LPS) (10 ng/mL) (Sigma, Stockholm, Sweden) was added as a positive control for activation/maturation of DCs. For confocal microscopy analysis, PBMCs and DCs were, before co-culture, labelled with green fluorescent dye PKH67 (Sigma) and red fluorescent dye PKH26 (Sigma) respectively. Labelling was performed according to manufacturer's protocol. Cytochalasin D (Sigma) (0.5 μg/ml) was added to co-cultures as a negative control for phagocytosis.

### Phenotypic Characterization of DC and PBMC

DCs were washed and resuspended in PBS with 2% FBS. They were incubated for 30 min in 4° C. with the following anti-human monoclonal antibodies: CD1a (clone NA1/34, DAKO, Glostrup, Denmark), CD14 (clone TÜK4; DAKO), CD19 (clone HD37, DAKO), CD3 (clone SK7), CD80 (clone L307.4), CD83 (clone HB15e), CD86 (clone 2331/FUN-1) HLA-DR (clone L243; all from BD Biosciences, San Diego, Calif.). PBMCs were washed and incubated with anti-human monoclonal antibodies, CD3 (clone SK7), CD4 (clone SK3), CD8 (clone G42-8), CD154 (clone TRAP-1), CD25 (clone 2A3) and CD69 (FN50; all from BD). Cell surface expression was measured by a FACScalibur flow cytometer (Becton Dickinson) and at least 10⁵ cells/sample were collected. Co-culture samples were at 72 hours washed and incubated with the previously mentioned CD1a, CD4, CD8, CD80, CD83, CD86 and HLA-DR. For analysis of DCs gates were set on CD41CD8⁻ or CD3⁻, CD1a⁺ cells.

### Cytokine/Chemokine Production

Supernatants from co-cultures or irradiated PBMC alone were analysed for cytokine/chemokine content by using a Bio-Plex assay (Biosource, Nivelles, Belgium). The assay was used according to manufacturer's protocol and a Luminex reader (Luminex Corporation, Austin Tex., USA) was used to simultaneously quantify the concentration of IL-6, IL-8, IL-2, IL-10, IL-12p70, TNFα, IFNγ, and MIP-1β in the supernatants.

Autologous T-Cell Proliferation and Activation

Immature DCs were obtained as above. Blood from the same donors were used for separation of CD3⁺ T-cells by negative selection using RosetteSep's Human CD3⁺ T cell Enrichment (1 mL/10 mL blood; Stem Cell Technologies). T-cells were frozen in 10% DMSO. On day 6 of DC culture, DC/apoptotic cell co-cultures were set up as above and were incubated for 48 h. A control consisting of DCs co-cultured with aCD8 (clone SK1, BD) (4 μg/ml) treated, apoptotic PBMC was also included. After co-incubation autologous T-cells were thawed, washed three times in RPMI and labelled with CFSE as described (38). 1.5×10⁶ T-cells were added to the corresponding DC donor in 10:1 proportion or to controls containing apoptotic cells only to a total volume of 1.5 mL. In positive controls staphylococcal enterotoxin B (SEB) (Sigma) (5 g/ml) was added. These cultures were incubated for 3, 4, 5 or 6 days. Brefeldin A (BFA) (Sigma) (10 μg/ml) was added to cultures 12 hours before staining for surface markers CD1a and CD3 and intracellular IFNγ (clone 25723.11, BD). Cells were first incubated with mAb directed against cell surface markers as described above. For intracellular staining cells were fixed in 2% formaldehyde, washed in saponin buffer consisting of 2% FBS, 2% HEPES, Saponin 1 mg/ml in PBS and were incubated with antibody at 4° C. for 30 min. Cells were finally washed in saponin buffer and analysed by FACS for cell-surface expression, proliferation and IFNγ expression. Gates were set on CD1a⁻, CD3⁺ cells.

### Statistical Analysis

Statistical significance was assessed using unpaired t tests and differences were considered significant at p≦0.05.

### Results

### Immature, Monocyte-Derived DCs Ingest Apoptotic PBMCs

Human monocytes were cultured for 6 days in presence of IL-4 and GM-CSF to obtain immature DCs as defined by expression of CD1a, lack of CD14 and low expression of the co-stimulatory molecules CD80, CD83 and CD86. We first determined whether the monocyte-derived, immature DCs had the ability to ingest apoptotic cells. PKH26 labelled immature DCs were co-cultured with PKH67 labelled apoptotic PBMCs. Confocal microscopy analyses were performed after 1, 4 or 24 hours of co-culture. We could not detect uptake of apoptotic cells after 1 hour while after 4 hours, uptake of apoptotic cells by DCs were detected as large, red/green double positive cells (FIG. 9a). Intracellularly localized apoptotic bodies were visualized in DCs after 4 hours of co-culture (FIG. 9b). After 24 hours of incubation the intensity of the double staining was increased and the DCs were enlarged, showing an increased uptake (FIG. 9c). At this time point intact apoptotic bodies were no longer detectable intracellularly. Cytochalasin D, which interferes with the phagocytic process by disruption of actin filaments (39) (40), was added to DCs together with the irradiated PBMCs and used as a negative control. DCs were harvested after 24 h and very few double positive cells were detected in the cultures containing cytochalasin D (FIG. 9d). This suggests that uptake of apoptotic PBMCs occurs via a phagocytic pathway because inhibition of actin filaments with cytochalasin D interferes with phagocytosis but leaves endocytic capacity intact (40). These results show that human monocyte-derived, immature DCs have the ability to phagocytose γ-irradiated PBMC and that large pieces of phagocytosed material can be detected within the cells.

### Activated, but not Resting, Apoptotic PBMCs Induce Expression of Co-Stimulatory Molecules in DCs

To investigate whether activated apoptotic T-cells have the capacity to provide activation/maturation signals to DCs, we first determined the efficiency of T cell activation by analyzing induction of CD25 and CD69 expression after activation with PHA or anti-CD3 and anti-CD28 mAbs (aCD3aCD28 activation) (FIG. 10). Both PHA and aCD3aCD28 activation resulted in up-regulation of CD25 and CD69. The frequency of positive cells did not differ notably between the different stimuli. T-cells were also stained for CD40L expression because CD40-CD40L interactions can induce DC maturation. CD40L expression could be detected in purified, activated T-cells, but not in the T-cell population present in PBMCs (data not shown). This is most likely due to the previously reported B-cell mediated endocytosis of CD40L on activated T-cells (41) (42). Non-activated and activated PBMC preparations were irradiated and apoptosis induction was measured by Annexin-V and PI stainings that were quantified by flow cytometry (FIG. 11). We show that both non-activated and activated PBMCs contain cells in early apoptosis and secondary necrosis. After 24 hours the majority of cells were double positive for Annexin-V and PI, which indicates that the γ-irradiation effectively induces apoptotic cell death in both resting and activated PBMCs.

Apoptotic PBMCs were added to immature DCs and the co-cultures were incubated for 72 h. To exclude the possibility that activation occurred via antibody binding to Fc-receptors on DCs, aCD3 and aCD28 antibodies were added in control DC cultures. Cells were collected and stained for CD1a, CD80, CD83, CD86 and HLA-DR and subjected to flow cytometric analyses. Mature DCs were defined as CD1a+ cells with distinct, high expression of CD86. Quadrants were set based on negative controls (medium) and positive controls (LPS). There was a significant increase in the frequency of CD86 expressing DCs as compared to the medium control in co-cultures containing activated PBMCs. Resting apoptotic cells or antibodies did not induce significant CD86 expression in DCs (FIG. 12a). A tendency towards a stronger induction of CD86 using αCD3αCD28 activated apoptotic cells was observed. For this reason and the fact that antibody induced activation can be used in GMP approved settings, we used αCD3αCD28 mAbs to activate PBMCs in the majority of subsequent experiments. Mean fluorescence intensity (MFI) values for CD80-, CD83-, CD86- and HLA-DR expression on DCs co-cultured with non-activated or αCD3αCD28 activated apoptotic cells were compared with medium control (FIG. 12b). The expression of CD80, CD83 and CD86 molecules were up-regulated in DCs co-cultured with activated apoptotic cells while HLA-DR expression did not differ significantly from the medium control. Purified, αCD3αCD28 activated, apoptotic CD4⁺ T-cells were also able to induce expression of co-stimulatory molecules in DCs (data not shown). These results show that activated, but not resting, apoptotic PBMCs are potent inducers of DC maturation as defined by up-regulation of co-stimulatory molecules.

### Resting, Necrotic PBMCs do not Induce DC Maturation

To examine the possibility that it was necrotic cells present in the samples exposed to γ-irradiation that caused maturation of DCs we compared the state of maturation in DCs co-cultured with resting or αCD3αCD28 activated γ-irradiated PBMCs as well as resting or activated freeze-thawed necrotic PBMCs. We detected no significant up-regulation of CD86 expression in DCs co-cultured with resting, necrotic or apoptotic cells while both the activated apoptotic and necrotic cells induced significant CD86 expression as compared to medium control. The activated apoptotic cells were however more potent inducers of DC maturation as compared to the necrotic PBMCs (FIG. 13). These results demonstrate that the presence of necrotic cells in the apoptotic cell preparations cannot solely explain induction of DC maturation.

Supernatants from Activated, Apoptotic PBMCs do not Induce DC Maturation

We next investigated whether the up-regulation of co-stimulatory molecules in DCs after co-culture with activated, irradiated PBMCs was due to extra-cellular factors released by the apoptotic cells. Supernatants from αCD3αCD28 activated, irradiated PBMCs were therefore collected after 4, 8 and 24 hours. DCs co-cultured with activated PBMCs significantly up-regulated CD86 expression, while supernatants from the same apoptotic cell preparations, were not effective in inducing CD86 expression (FIG. 14). This indicates that interaction between DCs and activated apoptotic cells is required for induction of DC maturation. It does however not exclude the possibility that extra-cellular factors released from apoptotic cells in a close proximity to, or within the DC can mediate up-regulation of co-stimulatory molecules.

### Activated Apoptotic PBMC Induce Pro-Inflammatory Cytokine Release in DC

To further analyse DC activation after addition of activated apoptotic cells we studied the cytokine and chemokine production in the DCs. Immature DCs were co-cultured with non-activated, apoptotic PBMCs, apoptotic PBMCs activated with PHA over night or for 4 days or apoptotic PBMCs activated with aCD3 and αCD28 antibodies over night. Supernatants were collected after 4, 8 and 24 hours from DC/apoptotic cell co-cultures. The supernatants were frozen and later analysed by luminex for IL-2, IL-6, IL-8, IL-10, IL-12, IFNγ, TNFα and MIP-1β content. There was a significant release of IL-6, TNFα and MIP-1β in the co-cultures containing DCs and activated apoptotic cells which was detected already at early time points. Significantly lower levels of these cytokines were detected in supernatants collected from apoptotic cells alone, suggesting production and release from the DCs. In most cases αCD3αCD28 activated apoptotic cells induced the highest levels of cytokines and also the most rapid release from DC. Supernatants from DCs co-cultured with non-activated apoptotic cells did not contain cytokine levels above levels detected in the medium control (FIG. 15). Irradiated neutrophils were also co-cultured with DCs but these did not induce any detectable cytokine release (data not shown). IL-2, IL-8 and IFNγ were detected in supernatants from co-cultures containing activated apoptotic cells. However, due to high release of these cytokines from activated apoptotic cells per se, it was not possible to attribute the production to the DCs (data not shown). We could not detect release of neither IL-10 nor IL-12 in any of the DC/apoptotic cell co-cultures examined (data not shown). The results show that DCs produce pro-inflammatory cytokines and chemokines after interaction with activated, apoptotic PBMCs. However, these DCs failed to produce IL-10 and IL-12.

DCs that Ingest Allogeneic, Activated, Apoptotic PBMCs Stimulate Proliferation and IFN-γ Production in Autologous T-cells

We next asked the question whether DCs matured by activated, apoptotic PBMCs are able to induce proliferation and activation of naïve T-cells. Autologous T-cells were added to DCs that had ingested either non-activated or activated allogeneic, apoptotic PBMC. The DCs/apoptotic cells co-cultures were incubated for 48 hours before addition of autologous, CFSE labelled T-cells. CFSE labelled T-cells alone or T-cells added to activated apoptotic cells were used as negative controls. As a positive control, the superantigen SEB was added to DCs together with autologous T-cells. Cultures were incubated for 3, 4, 5 or 6 days to determine the peak of T-cell proliferation. At these time points cells were collected and stained for CD1a and CD3 as well as intracellular IFNγ production. Samples were analysed by flow cytometry and gates were set on CD1a⁻, CD3⁺ cells. In the wells containing only DCs and autologous T cells, T-cells only, T-cells and activated apoptotic cells but no DCs or in samples where DCs were fed resting apoptotic cells, neither T-cell proliferation nor IFNγ production were detected at any of the time points analysed. In the SEB stimulated control proliferation peaked at day 4 which coincided with the highest frequency of IFNγ positive T-cells. DCs co-cultured with activated apoptotic cells before addition of T-cells were capable of inducing both proliferation and IFNγ production in autologous T-cells. As in the SEB control, both proliferation and IFNγ production peaked at day 4 (FIG. 16). To control for possible FcR-mediated effects on DC maturation and induction of efficient antigen presenting capacity, PBMCs were incubated with anti-CD8 antibody and exposed to γ-irradiation before addition to DCs. Anti-CD8 did not activate the T-cells as measured by up-regulated CD25 and CD69, and did not provide induction of DC activation and subsequent autologous T-cell proliferation (data not shown). Due to limitations of the four-colour flow cytometer the analysis included the total CD3⁺ T-cell population and different CD4/CD8 T-cell subsets could not be analysed. These results show that activated, but not resting, allogeneic FBMCs are able to induce DCs maturation that leads to efficient presentation of allo-antigens to T-cells.

### Discussion

The mechanisms for induction of DC activation and subsequent priming of an adaptive immune response are not fully clarified. Conserved microbial and viral patterns binding to PPRs on DCs have been shown as effective mediators of adaptive immune responses. These are however not the answer to why material lacking PPR affinity is able to initiate immune responses. Our study demonstrates that activated, but not resting, apoptotic PBMCs are able to induce activation of DCs in terms of up-regulation of co-stimulatory molecules, induction of pro-inflammatory cytokine release and presentation of allo-antigens that lead to T-cell proliferation and IFNγ production. Necrotic cells were also able to induce DC maturation to some degree if initially activated, but failed to do so in absence of preceding stimuli. The present report supports earlier studies where DCs exposed to apoptotic cells were found to mature and induce activation of T-cells in vitro (30, 31, 33, 43-45) and that the activated apoptotic cells are more efficient than activated necrotic cells in this aspect (46, 47). The dying cells inducing DC activation in the former studies all contained different forms of tumor- or viral antigens. The danger signalling features of these cells are still not fully characterized but we speculate that the effect of the apoptotic cells partly could be associated with a “non-resting” state. It should be noted that no TLR-ligand-, tumour- or viral source of antigen was present in the setup of our experiments. We here suggest that the state of activation, before a cell enters apoptosis, is what determines its ability to activate DCs. However, the induction of “endogenous” TLR-ligand expression in activated T cells cannot be excluded. The use of resting apoptotic cells in previous experiments could partly explain why some studies have found apoptotic cells unable to mature DCs in vitro (10, 22) to possess anti-inflammatory properties (24-26, 48) or to induce tolerance instead of immune activation ((27, 49, 50).

Some endogenous factors originating from dying cells have been suggested as plausible effectors of DC activation. HSPs have earlier been shown to induce DC maturation ((51-59) and exert adjuvant activity (60-63). These molecules are intracellular and released upon lost membrane integrity. HSPs could possibly have some effect in our in-vitro system where some of the irradiated PBMC most likely enter secondary necrosis before uptake of DCs. Yet this is not a fully satisfying explanation of our results for two reasons. First, supernatants collected from apoptotic cells at later time points also contain factors released from cells in secondary necrosis. These supernatants lacked the ability to induce DC maturation. Secondly, comparing apoptotic and necrotic cells, the latter were less efficient in up-regulating co-stimulatory molecules on DCs. Another endogenous molecule that was suggested to activate DCs is uric acid, which is the end product in purine degradation and present in high amounts in stressed cells. Uric acid is contained in the cytosol of cells and not accessible to surrounding cells unless membrane integrity is lost why we exclude this as the main effector of DC activation. The essential factor or factors in activated apoptotic cells with ability to initiate DC activation remains to be elucidated.

The present study shows that exposure to activated apoptotic cells induce production of pro-inflammatory cytokines in DCs. We could however not detect any release of IL-12, important in eliciting Th1 responses and counteracting tolerogenic responses. Uptake of apoptotic cells has previously been shown to down-regulate LPS-induced IL-12 production. (64) However, it remains to be elucidated whether this is a reversible effect. The apparent lack of IL-12 production in the DC/activated apoptotic cell cultures may be explained by lack of a secondary signal, which can be provided by CD40 ligation (65). We suggest that the CD40L signal may instead be delivered by the autologous T-cells after antigen recognition (FIG. 16). It is likely that the naïve T-cells up-regulate CD40L in response to allo-antigen presentation and co-stimulatory molecule stimulation by DCs.

In an inflammatory event, caused by pathogens or injury of host cells, immune cells are recruited to the sight for elimination of potential danger. The recruited cells may become activated and after carrying out their mission many cells die by apoptosis. We speculate that this form of apoptosis could function as a positive feed-back mechanism for both the innate and adaptive response in an inflammatory event. When immature DCs, residing at the sight of infection or injury, take up activated apoptotic cells, pro-inflammatory cytokines are released. This would increase the recruitment of immune cells to the sight. DCs phagocytosing activated apoptotic cells are also able to up-regulate co-stimulatory molecules. When the mature DCs migrate to draining lymphnodes, antigen can be presented to naïve T-cells thereby ensuring that the activated peripheral lymphocyte do not die in vain without alerting the immune system. Resting cells that dye by apoptosis, on the other hand, lack this effect on DCs, which would explain why the frequent turnover of cells during “normal” conditions occurs without alarming the immune system.

The endogenous adjuvant effect attributed to activated apoptotic cells reported here could also be of relevance for rational design of vaccines. Several of the vectors currently under development induce apoptosis in their target cell, which may subsequently lead to cross-presentation of antigens. We speculate that the cross-presentation pathway may be further augmented if the vector used is also able to induce activation in the target cell enabling co-delivery of antigen and endogenous adjuvant. The recent finding that efficient antigen presentation of phagocytosed cargo is dependent upon TLR ligands within the cargo, would support the hypothesis that adjuvant should be co-delivered with the apoptotic material.

Taken together, this study demonstrates that apoptotic cells have different abilities to elicit immune responses depending on their state of activation, and also indicates apoptotic cells could be used for development of vaccines that utilize cross-presentation of apoptotic cells.

### REFERENCES

1. Banchereau, J., F. Briere, C. Caux, J. Davoust, S. Lebecque, Y. J. Liu, B. Pulendran, and K. Palucka. 2000. Immunobiology of dendritic cells. Annu Rev Immunol 18:767-811.
2. Banchereau, J., and R. M. Steinman. 1998. Dendritic cells and the control of immunity. Nature 392:245-252.
3. Guermonprez, P., J. Valladeau, L. Zitvogel, C. Thery, and S. Amigorena. 2002. Antigen presentation and T cell stimulation by dendritic cells. Annu Rev Immunol 20:621-667.
4. de Jong, E. C., P. L. Vieira, P. Kalinski, J. H. Schuitemaker, Y. Tanaka, E. A. Wierenga, M. Yazdanbakhsh, and M. L. Kapsenberg. 2002. Microbial compounds selectively induce Th1 cell-promoting or Th2 cell-promoting dendritic cells in vitro with diverse th cell-polarizing signals. J Immunol 168:1704-1709.
5. Kalinski, P., C. M. Hilkens, E. A. Wierenga, and M. L. Kapsenberg. 1999. T-cell priming by type-1 and type-2 polarized dendritic cells: the concept of a third signal. Immunol Today 20:561-567.
6. Sporri, R., and C. Reis e Sousa. 2005. Inflammatory mediators are insufficient for full dendritic cell activation and promote expansion of CD4⁺ T cell populations lacking helper function. Nat Immunol 6:163-170.
7. Janeway, C. A., Jr, editor. 1989. Approaching the asymptote? Evolution and revolution in immunology. 1-13 pp.
8. Akira, S., and K. Takeda. 2004. Toll-like receptor signalling. Nat Rev Immunol 4:499-511.
9. Reis e Sousa, C. 2004. Toll-like receptors and dendritic cells: for whom the bug tolls. Semin. Immunol.: 27-34.
10. Gallucci, S., M. Lolkema, and P. Matzinger. 1999. Natural adjuvants: endogenous activators of dendritic cells. Nat Med 5:1249-1255.
11. Heufler, C., F. Koch, and G. Schuler. 1988. Granulocyte/macrophage colony-stimulating factor and interleukin 1 mediate the maturation of murine epidermal Langerhans cells into potent immunostimulatory dendritic cells. J Exp Med 167:700-705.
12. Lebre, M. C., J. C. Antons, P. Kalinski, J. H. Schuitemaker, T. M. van Capel, M. L. Kapsenberg, and E. C. De Jong. 2003. Double-stranded RNA-exposed human keratinocytes promote Th1 responses by inducing a Type-1 polarized phenotype in dendritic cells: role of keratinocyte-derived tumor necrosis factor alpha, type I interferons, and interleukin-18. J Invest Dermatol 120:990-997.
13. Luft, T., K. C. Pang, E. Thomas, P. Hertzog, D. N. Hart, J. Trapani, and J. Cebon. 1998. Type I IFNs enhance the terminal differentiation of dendritic cells. J Immunol 161:1947-1953.
14. Luft, T., M. Jefford, P. Luetjens, T. Toy, H. Hochrein, K. A. Masterman, C. Maliszewski, K. Shortnan, J. Cebon, and E. Maraskovsky. 2002. Functionally distinct dendritic cell (DC) populations induced by physiologic stimuli: prostaglandin E(2) regulates the migratory capacity of specific DC subsets. Blood 100: 1362-1372.
15. Sallusto, P., and A. Lanzavecchia. 1994. Efficient presentation of soluble antigen by cultured human dendritic cells is maintained by granulocyte/macrophage colony-stimulating factor plus interleukin 4 and downregulated by tumor necrosis factor alpha. J Exp Med 179:1109-1118.
16. Blander, J. M., and R. Medzhitov. 2006. Toll-dependent selection of microbial antigens for presentation by dendritic cells. Nature.
17. Janeway, C. A., Jr. 1992. The immune system evolved to discriminate infectious nonself from noninfectious self. Immunol Today 13:11-16.
18. Matzinger, P. 1994. Tolerance, danger, and the extended family. Annu Rev Immunol 12:991-1045.
19. Rock, K. L., A. Hearn, C. J. Chen, and Y. Shi. 2005. Natural endogenous adjuvants. Springer Semin Immunopathol 26:231-246.
20. Jenne, L., J. F. Arrighi, H. Jonuleit, J. H. Saurat, and C. Hauser. 2000. Dendritic cells containing apoptotic melanoma cells prime human CD8+T cells for efficient tumor cell lysis. Cancer Res 60:4446-4452.
21. Sauter, B., M. L. Albert, L. Francisco, M. Larsson, S. Somersan, and N. Bhardwaj. 2000. Consequences of cell death: exposure to necrotic tumor cells, but not primary tissue cells or apoptotic cells, induces the maturation of immunostimulatory dendritic cells. J Exp Med 191:423-434.
22. Somersan, S., M. Larsson, J. F. Fonteneau, S. Basu, P. Srivastava, and N. Bhardwaj. 2001. Primary tumor tissue lysates are enriched in heat shock proteins and induce the maturation of human dendritic cells. J Immunol 167:4844-4852.
23. Fadok, V. A., D. L. Bratton, and P. M. Henson. 2001. Phagocyte receptors for apoptotic cells: recognition, uptake, and consequences. J Clin Invest 108:957-962.
24. Morelli, A. E., A. T. Larregina, W. J. Shufesky, A. F. Zahorchak, A. J. Logar, G. D. Papworth, Z. Wang, S. C. Watkins, L. D. Falo, Jr., and A. W. Thomson. 2003. Internalization of circulating apoptotic cells by splenic marginal zone dendritic cells: dependence on complement receptors and effect on cytokine production. Blood 101:611-620.
25. Savill, J., I. Dransfield, C. Gregory, and C. Haslett. 2002. A blast from the past: clearance of apoptotic cells regulates immune responses. Nat Rev Immunol 2:965-975.
26. Stuart, L. M., M. Lucas, C. Simpson, J. Lamb, J. Savill, and A. Lacy-Hulbert. 2002. Inhibitory effects of apoptotic cell ingestion upon endotoxin-driven myeloid dendritic cell maturation. J Immunol 168:1627-1635.
27. Ferguson, T. A., J. Herndon, B. Elzey, T. S. Griffith, S. Schoenberger, and D. R. Green. 2002. Uptake of apoptotic antigen-coupled cells by lymphoid dendritic cells and cross-priming of CD8(+) T cells produce active immune unresponsiveness. J Immunol 168:5589-5595.
28. Steinman, R. M. 2001. Dendritic cells and the control of immunity: enhancing the efficiency of antigen presentation. Mt Sinai J Med 68:106-166.
29. Buttiglieri, S., A. Galetto, S. Fomo, M. De Andrea, and L. Matera. 2003. Influence of drug-induced apoptotic death on processing and presentation of tumor antigens by dendritic cells. Int J Cancer 106:516-520.
30. Feng, H., Y. Zeng, M. W. Graner, and E. Katsanis. 2002. Stressed apoptotic tumor cells stimulate dendritic cells and induce specific cytotoxic T cells. Blood 100:4108-4115.
31. Goldszmid, R. S., J. Idoyaga, A. I. Bravo, R. Steinman, J. Mordoh, and R. Wainstok. 2003. Dendritic cells charged with apoptotic tumor cells induce long-lived protective CD4+ and CD8+T cell immunity against B16 melanoma. J Immunol 171:5940-5947.
32. Hoffmann, T. K., N. Meidenbauer, G. Dworacki, H. Kanaya, and T. L. Whiteside. 2000. Generation of tumor-specific T-lymphocytes by cross-priming with human dendritic cells ingesting apoptotic tumor cells. Cancer Res 60:3542-3549.
33. Ignatius, R., M. Marovich, E. Mehlhop, L. Villamide, K. Mahnke, W. I. Cox, F. Isdell, S. S. Frankel, J. R. Mascola, R. M. Steinman, and M. Pope. 2000. Canarypox virus-induced maturation of dendritic cells is mediated by apoptotic cell death and tumor necrosis factor alpha secretion. J Virol 74:11329-11338.
34. Ishii, S., K. Hiroishi, J. Eguchi, and K. Mitamura. 2003. Dendritic cell maturation induced by delivery of ultraviolet-mediated apoptotic colorectal cancer cell lines. Anticancer Res 23:2457-2463.
35. Spetz, A. L., A. S. Sorensen, L. Walther-Jallow, B. Wahren, J. Andersson, L. Holmgren, and J. Hinkula. 2002. Induction of HIV-1-specific immunity after vaccination with apoptotic HIV-1/murine leukemia virus-infected cells. J Immunol 169:5771-5779.
36. Holmgren, L., A. Szeles, E. Rajnavolgyi, J. Folkman, G. Klein, I. Ernberg, and K. I. Falk. 1999. Horizontal transfer of DNA by the uptake of apoptotic bodies. Blood 93:3956-63.
37. Spetz, A. L., B. K. Patterson, K. Lore, J. Andersson, and L. Holmgren. 1999. Functional gene transfer of HIV DNA by an HIV receptor-independent mechanism. J Immunol 163:736-742.
38. Lyons, A. B., and C. R. Parish. 1994. Determination of lymphocyte division by flow cytometry. J Immunol Methods 171:131-137.
39. Cooper, J. A. 1987. Effects of cytochalasin and phalloidin on actin. J Cell Biol 105:1473-1478.
40. DeFife, K. M., C. R. Jenney, E. Colton, and J. M. Anderson. 1999. Disruption of filamentous actin inhibits human macrophage fusion. Faseb J 13:823-832.
41. Miyashita, T., M. J. McIlraith, A. C. Grammer, Y. Miura, J. F. Attrep, Y. Shimaoka, and P. E. Lipsky. 1997. Bidirectional regulation of human B cell responses by CD40-CD40 ligand interactions. J Immunol 158:4620-4633.
42. Yellin, M. J., K. Sippel, G. Inghirami, L. R. Covey, J. J. Lee, J. Sinning, F. A. Clark, L. Chess, and S. Lederman. 1994. CD40 molecules induce down-modulation and endocytosis of T cell surface T cell-B cell activating molecule/CD40-L. Potential role in regulating helper effector function. J Immunol 152:598-608.
43. Rad, A. N., G. Pollara, S. M. Sohaib, C. Chiang, B. M. Chain, and D. R. Katz. 2003. The differential influence of allogeneic tumor cell death via DNA damage on dendritic cell maturation and antigen presentation. Cancer Res 63:5143-5150.
44. Rovere, P., C. Vallinoto, A. Bondanza, M. C. Crosti, M. Rescigno, P. Ricciardi-Castagnoli, C. Rugarli, and A. A. Manfredi. 1998. Bystander apoptosis triggers dendritic cell maturation and antigen-presenting function. J Immunol 161:4467-4471.
45. Rovere, P., M. G. Sabbadini, C. Vallinoto, U. Fascio, V. S. Zimmermarm, A. Bondanza, P. Ricciardi-Castagnoli, and A. A. Manfredi. 1999. Delayed clearance of apoptotic lymphoma cells allows cross-presentation of intracellular antigens by mature dendritic cells. J Leukoc Biol 66:345-349.
46. Scheffer, S. R., H. Nave, F. Korangy, K. Schlote, R. Pabst, E. M. Jaffee, M. P. Manns, and T. F. Greten. 2003. Apoptotic, but not necrotic, tumor cell vaccines induce a potent immune response in vivo. Int J Cancer 103:205-211.
47. Schnurr, M., C. Scholz, S. Rothenfusser, P. Galambos, M. Dauer, J. Robe, S. Endres, and A. Eigler. 2002. Apoptotic pancreatic tumor cells are superior to cell lysates in promoting cross-priming of cytotoxic T cells and activate NK and gammadelta T cells. Cancer Res 62:2347-2352.
48. Huynh, M. L., V. A. Fadok, and P. M. Henson. 2002. Phosphatidylserine-dependent ingestion of apoptotic cells promotes TGF-beta1 secretion and the resolution of inflammation. J Clin Invest 109:41-50.
49. Ferguson, T. A., and H. Kazama. 2005. Signals from dying cells: tolerance induction by the dendritic cell. Immunol Res 32:99-108.
50. Steinman, R. M., S. Turley, I. Mellman, and K. Inaba. 2000. The induction of tolerance by dendritic cells that have captured apoptotic cells. J Exp Med 191:411-416.
51. Basu, S., R. J. Binder, R. Suto, K. M. Anderson, and P. K. Srivastava. 2000. Necrotic but not apoptotic cell death releases heat shock proteins, which deliver a partial maturation signal to dendritic cells and activate the NF-kappa B pathway. Int Immunol 12:1539-1546.
52. Bethke, K., F. Staib, M. Distler, U. Schmitt, H. Jonuleit, A. H. Enk, P. R. Galle, and M. Heike. 2002. Different efficiency of heat shock proteins (HSP) to activate human monocytes and dendritic cells: superiority of HSP60. J Immunol 169:6141-6148.
53. Flohe, S. B., J. Bruggemann, S. Lendemans, M. Nikulina, G. Meierhoff, S. Flohe, and H. Kolb. 2003. Human heat shock protein 60 induces maturation of dendritic cells versus a Th1-promoting phenotype. J Immunol 170:2340-2348.
54. Kuppner, M. C., R. Gastpar, S. Gelwer, E. Nossner, O. Ochmann, A. Scharner, and R. D. Issels. 2001. The role of heat shock protein (hsp70) in dendritic cell maturation: hsp70 induces the maturation of immature dendritic cells but reduces DC differentiation from monocyte precursors. Eur J Immunol 31:1602-1609.
55. Massa, C., C. Melani, and M. P. Colombo. 2005. Chaperon and adjuvant activity of hsp70: different natural killer requirement for cross-priming of chaperoned and bystander antigens. Cancer Res 65:7942-7949.
56. Millar, D. G., K. M. Garza, B. Odermatt, A. R. Elford, N. Ono, Z. Li, and P. S. Ohashi. 2003. Hsp70 promotes antigen-presenting cell function and converts T-cell tolerance to autoimmunity in vivo. Nat Med 9:1469-1476.
57. Singh-Jasuja, H., H. U. Scherer, N. Hilf, D. Arnold-Schild, H. G. Rammensee, R. E. Toes, and H. Schild. 2000. The heat shock protein gp96 induces maturation of dendritic cells and down-regulation of its receptor. Eur J Immunol 30:2211-2215.
58. Singh-Jasuja, H., N. Hilf, H. U. Scherer, D. Arnold-Schild, H. G. Rammensee, R. E. Toes, and H. Schild. 2000. The heat shock protein gp96: a receptor-targeted cross-priming carrier and activator of dendritic cells. Cell Stress Chaperones 5:462-470.
59. Andersson, J., S. Kinloch, A. Sönnerborg, J. Nilsson, T. E. Fehniger, A. Spetz, H. Behbahani, L. Goh, H. McDade, B. Gazzard, H. Stellbrink, D. Cooper, and L. Perrin. 2002. Low perforin expression in CD8+ T. lymphocytes granules in lymphoid tissue during acute HIV-1 infection. J Infect Dis.
60. Srivastava, P. K., and M. Heike. 1991. Tumor-specific immunogenicity of stress-induced proteins: convergence of two evolutionary pathways of antigen presentation? Semin Immunol 3:57-64.
61. Udono, H., and P. K. Srivastava. 1993. Heat shock protein 70-associated peptides elicit specific cancer immunity. JExp Med 178:1391-1396.
62. Wan, T., X. Zhou, G. Chen, H. An, T. Chen, W. Zhang, S. Liu, Y. Jiang, F. Yang, Y. Wu, and X. Cao. 2004. Novel heat shock protein Hsp70L1 activates dendritic cells and acts as a Th1 polarizing adjuvant. Blood 103:1747-1754.
63. Wang, X. H., Y. Qin, M. H. Hu, and Y. Xie. 2005. Dendritic cells pulsed with gp96-peptide complexes derived from human hepatocellular carcinoma (HCC) induce specific cytotoxic T lymphocytes. Cancer Immunol Immunother 54:971-980.
64. Kim, S., K. B. Elkon, and X. Ma. 2004. Transcriptional suppression of interleukin-12 gene expression following phagocytosis of apoptotic cells. Immunity 21:643-653.
65. Snijders, A., P. Kalinski, C. M. Hilkens, and M. L. Kapsenberg. 1998. High-level IL-12 production by human dendritic cells requires two signals. Int Immunol 10:1593-1598.

### Example C

### Materials and Methods

### In Vitro Differentiation of Dendritic Cells (DCs)

CD14⁺ monocytes were enriched from peripheral blood mononuclear cells (PBMCs) from healthy blood donors by negative selection using RosetteSep Human Monocyte Enrichment (1 mL/10 mL blood; Stem Cell Technologies, Vancouver, BC, Canada). Monocytes were separated using lymphoprep (Nycomed, Oslo, Norway) density gradient. Cells were cultured for 6 days in medium (RPMI 1640 supplemented with 1% HEPES [N-2-hydroxyethylpiperazine-N′-2-ethanesulfonic acid], 2 mM L-glutamine, 1% Streptomycin and penicillin, 10% endotoxin-free fetal bovine serum (FBS); GIBCO Life Technologies, Paisley, United Kingdom) and recombinant human cytokines IL-4 (6.5 ng/mL; R&D Systems, Minneapolis, Minn.) and granulocyte macrophage-colony-stimulating factor (GM-CSF; 250 ng/mL; Peprotech, London, UK), to obtain immature dendritic cells.

### Activation of T Cells

CD4⁺ and CD8⁺ T cells were enriched from healthy blood donor PBMCs by negative selection using RosetteSep's Human CD4⁺ or CD8⁺ T cell Enrichment (1 mL/10 mL blood respectively; Stem Cell Technologies). T cells were separated using lymphoprep density gradient (Nycomed, Oslo, Norway). Cells were frozen in FBS and 10% dimethylsulphoxide (DMSO) or were added to flasks containing 1% Sodiumpyruvate, monoclonal anti-human CD3 (2 μg/ml; clone OKT 3; Ortho Biotech Inc. Raritan, N.J.), that was adhered to the plastic during one hour in 4° C., and soluble monoclonal anti-human CD28 (2 μg/ml; L293; BD Biosciences, San Diego, Calif.). After stimulation cells were frozen in FBS/DMSO.

### HIV-1 Virus Growth and Preparation

The CCR5-using HIV-1_{BaL} isolate or CXCR4 HIV-1_{IIIB} (National Institutes of Health (NIH) AIDS Research and Reference Reagent Program, Division of AIDS, National Institute of Allergy and Infectious Diseases (NIAID), NIH) was grown on PBMC cultures stimulated with PHA (Sigma, St Louis, Mo.) and IL-2 (Chiron, Emeryville, Calif.). To concentrate the virus and to minimize the presence of bystander activation factors in the supernatant that could induce DC maturation, the virus was ultracentrifuged (138 000 g (45 000 rpm), 30 minutes, 4° C., Beckman L-80 Ultracentrifuge, rotor 70.1; Beckman Coulter, Fullerton, Calif.) and the virus pellet was resuspended in RPMI 10% FBS to obtain a 10× virus concentrate. The viral titer of the HIV-1_{BaL} stock was determined by p24 enzyme-linked immunosorbent assay (ELISA; Murez HIV antigen Mab; Abbott, Abbott Park, Ill.) according to manufacturer's protocol. Samples were analyzed in serial dilutions in duplicate. The 10×HIV-1_{BaL} stock had an HIV-1 p24 Gag content of 11.7 μg/mL. The HIV-1_{BaL} stock was also characterized by determining the level of active reverse transcriptase (RT; Lenti RT; Cavidi Tech, Uppsala, Sweden). The 10×HIV-1 BaL stock used contained 15 000 pg active RT/mL.

### HIV-1 Infection of T Cells and Dendritic Cells

CD4+ T cells were isolated from healthy blood donor PBMCs by negative selection using RosetteSep's Human CD4⁺ T cell Enrichment (1 mL/10 mL blood respectively; Stem Cell Technologies) and activated with anti-CD3 (2 μg/ml; clone OKT 3; Ortho Biotech Inc. Raritan, N.J.) and anti-CD28 mAb (2 μg/ml; L293; BD Biosciences, San Diego, Calif.) over night. The cells were then incubated with 10×HIV-1_{BaL} or 1×HIV-1_{BaL} stocks (200 μl HIV-1_{BaL} stock to 1×10⁶ CD4⁺ T cells) in the presence of IL-2 (Chiron, Emeryville, Calif.). The frequency of infected cells was analyzed by intracellular p24 staining day 3, 4, 5, 6, 7 and 10 after infection. The obtained infected cells were frozen in FBS/DMSO until use. A quantity of 200 μL of 1×HIV-1_{BaL} or mock was added to 5×10⁵ immature DCs/mL in a 24-well plate (Costar Corning, Corning, N.Y.) to a final volume of 1.0 mL per well. The frequency of infected DCs was determined by intracellular p24 staining after 72 hours and 7 days of infection.

### Quantification of HIV-1 Protein in T Cells and Dendritic Cells

The frequency of HIV-1_{BaL} infection in DCs and T cells was determined by intracellular staining for the HIV-1 Gag protein p24. Cells were first stained for cell surface markers, then washed in PBS and fixed in 2% formaldehyde (Sigma) for 10 minutes at room temperature. Cells were washed in PBS with 2% FBS followed by a wash in PBS with 2% FBS, 2% HEPES and 0.1% Saponin (Sigma) to allow permeabilization of the cell surface membrane. Cells were incubated for 1-2 hour at 4° C. with the anti-p24 specific mAb (clone KC57; Coulter, Hialeah, FL) or the corresponding isotype control. Cells were washed in saponin solution to remove excessive antibody and resuspended in PBS. Expression was assessed by a FACSCalibur flow cytometer (Becton Dickinson).

### Production of Apoptotic Cells and Apoptotic Cell Supernatants

Frozen T cells were thaw and washed 3 times in RPMI. Cells were induced to undergo apoptosis by γ-irradiation (150 Gy). The γ-irradiation induced apoptotic process has previously been demonstrated by morphological changes, flow cytometry and DNA fragmentation on agarose gels (Holmgren et al., 1999, Blood 93:3956; Spetz et al., 1999, J Immunol 163:736). Apoptosis was here confirmed by flow cytometry stainings with AnnexinV (Boehringer Mannheim, Mannheim, Germany) and propidium iodide (PI) (0.1 μg/sample; Sigma, Stockholm, Sweden) according to manufacturer's protocol. Supernatants were collected from live and irradiated cells after 4, 8 and 24 hours and were centrifuged at 1.4×10⁴ rpm for 30 min to remove possible cell debris.

### DC Co-Cultures

On day 6, immature DCs were counted and plated in 24-well plates, 5×10⁵ cells in 0.5 mL medium (RPMI supplemented with 10% FBS and recombinant human IL-4 and GM-CSF). Irradiated T cells were added to DCs in proportion 2:1 to a total volume of 1 mL. Supernatant (0.5 mL) from 106 irradiated T cells, collected at 4, 8 and 24 hours, was also added to immature DCs. Supernatant was collected from co-cultures at 4, 8 and 24 hours. At 72 hours or after 7 days all samples were collected and DCs were characterized by flow cytometric analysis. Lipopolysaccharide (LPS 100 ng/mL, Sigma) was added as a positive control for activation/maturation of DCs.

### Phenotypic Characterization of DCs and T Cells

DCs were washed and resuspended in PBS with 2% FBS. They were incubated for 30 min in 4° C. with the following anti-human monoclonal antibodies (mAbs): CD1a (clone NA1/34, DAKO, Glostrup, Denmark), CD14 (clone TÜK4; DAKO), CD19 (clone HD37, DAKO), CD3 (clone SK7), CD83 (clone HB15e) and CD86 (clone 2331/FUN-1; all from BD Biosciences, San Diego, Calif.). T cells were washed and incubated with anti-human monoclonal antibodies CD19 (clone HD37; DAKO), CD14 (DAKO), CD3 (clone SK7), CD4 (clone RPA-T4)+Streptavidin, CD8 (clone SK-1), CD154 (clone TRAP-1), CD25 (clone 2A3) and CD69 (FN50; all from BD). Cell surface expression was measured by a FACScalibur flow cytometer (Becton Dickinson) and at least 10⁵ cells/sample were collected. Co-culture samples were at 72 hours or 7 days washed and incubated with the previously mentioned CD1a, CD4, CD8, CD83 and CD86. DCs were also stained with Annexin V as in preceding paragraph to detect possible apoptotic DCs.

### Cytokine/Chemokine Production

Supernatants from irradiated T cells and co-cultures were analysed for cytokine/chemokine content by using a Bio-Plex assay (Biosource, Nivelles, Belgium). The assay was used according to manufacturer's protocol and a Luminex reader (Luminex Corporation, Austin Tex., USA) was used to simultaneously quantify the concentration of IL-6, IL-8, IL-2, IL-10, IL-12, TNFα, IFNγ, MIP-1α and MIP-1β in the supernatants.

### Results and Discussion

Up Regulation of Co-Stimulatory Molecules on Dendritic Cells after Co-Culture with Apoptotic HIV-1 Infected CD4⁺ T Cells.

To investigate whether activated apoptotic HIV-1 infected CD4⁺ T cells have the capacity to provide any activation/maturation signal to dendritic cells, we induced apoptosis in CD4⁺ T cells that were first activated anti-CD3 and anti-CD28 mAbs and thereafter infected with HIV-1 before adding them to human in vitro differentiated dendritic cells. The efficiency of T cell activation was determined by analyzing induction of CD25 and CD69 expression on T cells (FIG. 17A). We detected increased expression of both CD25 and CD69 molecules on CD4⁺ T cells after activation with anti-CD3/CD28 mAbs. These findings show that the T cells were efficiently activated in the culture system used. The kinetics of HIV-1 infection and a representative example of infection efficiency as determined by intracellular p24 staining is shown in FIG. 17B. Batches of cells containing 20-40% HIV-1 infected cells, as measured by intracellular p24 staining, were frozen and subsequently used to prepare apoptotic HIV-1 infected cells. The day of experiment, the frozen cells were thaw, washed and induced to undergo apoptosis by gamma-irradiation. Apoptosis induction was measured by performing Annexin-V and PI staining, which were quantified by flow cytometry as shown in Example B above.

To investigate whether apoptotic HIV-1 infected T cells may per se be able to induce maturation in DCs. We used in vitro differentiated monocytes cultured for 6 days in the presence of IL-4 and GM-CSF as source of human immature dendritic cells as defined by their expression of CD1a, lack of CD14 and low expression of CD40, CD80, CD86 and CD83. These immature dendritic cells were co-cultured with apoptotic cells for 72 hours or 7 days and then analyzed for expression of the co-stimulatory molecule CD86 (FIG. 18). Representative flow cytometric analyses are depicted in FIG. 18 and a summary of at least 11 donors are shown in FIG. 18 B. The frequency of CD86⁺ DCs was 91±2.5% after LPS stimulation and the background medium control was 27±5.3% after 75 hours. DCs exposed to HIV-1_{BaL} were not induced to express CD86 either at 72 hours or after 7 days of culture (FIG. 182B). However, co-culture with apoptotic activated either non-infected or HIV-1_{BaL} infected T cells resulted in significant induction of CD86 as compared to medium control both after 72 hours and 7 days of culture. The activation/maturation signal provided by the apoptotic activated CD4⁺ T cells occurred even in the presence of free HIV-1_{BaL}.

We also determined whether induction of CD83, which is another molecule that is associated with DC maturation and functional antigen-presenting capacity, was induced in the co-cultures. We could not detect significant induction of CD83 after exposure to HIV-1_{BaL}, while there was induction of CD83 after co-culture with apoptotic activated CD4⁺ T cells. The pattern of expression was similar to CD86 expression and CD83 was induced after co-culture with apoptotic activated CD4⁺ T cells even in the presence of HIV-1. These findings show that apoptotic activated CD4⁺ T cells are able to provide an activation/maturation signal to immature DCs even in the presence of HIV-1. Furthermore, a population of apoptotic activated T cells containing a high frequency of HIV-1 infected cells is also able to provide an activation/maturation signal to DCs. Altogether, these findings suggest that apoptotic activated T cells carrying HIV-1 may be used as an antigen transfer system that is able to induce certain DC activation/maturation, which turn DCs into highly efficient antigen-presenting cells.

The finding that apoptotic HIV-1 infected T cells are able to induce DC maturation has implications for viral transmission because mature DCs were demonstrated to be less susceptible to HIV-1 infection as compared to immature DCs. The DCs residing in the mucosa and are considered to be one of the first target cells during transmission and has an immature phenotype that resembles the cells used in the experiments described here. Hence, a composition that is able to induce DC maturation in monocyte derived dendritic cells has the potential to be able to induce maturation in mucosa associated DCs thereby shielding them from HIV-1 infection. It is therefore conceivable that apoptotic activated T cells could be used in a microbicide formulation whereby one mechanistic action would be to induce maturation in immature DCs.

Secretion of Pro-Inflammatory Cytokines after Co-Culture with Apoptotic Activated T Cells.

To address whether cytokine production was induced in DCs after uptake of apoptotic activated T cells, we collected supernatants from the co-cultures after 4, 8 and 24 hours (FIG. 19). The Luminex technology, which allows simultaneous analyses of up to eight cytokines, was used. There was a rapid induction of TNF-α, from the co-cultures with anti-CD3 and anti-CD28 activated cells either non-infected or HIV-1 infected apoptotic cells (FIG. 19A). We also detected IL-2 and IFN-γ in the co-cultures with DCs and apoptotic activated CD4⁺ T cells. We measured a rapid induction of MIB-1α and MIB-1β in the co-cultures with activated apoptotic T cells (both non-infected and HIV-1 infected) and there was no background secretion from the apoptotic cells per se (FIG. 19B). Co-culture with non-stimulated T cells or neutrophils did not result in any secretion of mentioned cytokines. Altogether, these findings suggest that activated apoptotic T cells are able to induce pro-inflammatory cytokine and chemokine production in DCs.

The finding that DCs produce chemokines with known anti-viral effect further supports the concept of using apoptotic activated T cells as a microbicide, where the anti-viral effect is achieved after interaction with DCs.

The production of certain pro-inflammatory cytokines and chemokines also support the use of apoptotic activated T cells as an antigen delivery system or additive in a vaccine to achieve local anti-viral activity upon therapeutic vaccination.

Reduced Frequency of HIV-1 Infected DCs after Co-Culture with Apoptotic Activated T Cells.

The finding that several cytokines were released into the supernatants, including those with anti-HIV-1 activity, prompted the question whether co-culture with apoptotic activated CD4⁺ T cells could influence the efficiency of virus infection in DCs. We measured the rate of HIV-1 infection by determining the frequency of cells expressing intracellular p24 antigen as previously described. Addition of 3′-azido-3′deoxythymidine (AZT) to the cultures inhibits detection of p24, suggesting that detected intracellular p24 in DCs is due to productive infection in DCs and not the result of uptake of viral particles or p 24 protein. In addition, the levels of HIV-1 p24 released in supernatants increase over time in the DC cultures exposed to HIV-1, as measured by ELISA (29).

Immature DCs were exposed to HIV-1_{BaL} and we found a large donor variability regarding HIV-1 infection efficiency ranging from 0.1-21.7% after 72 hours incubation and between 2.1-46.4% after 7 days. We could not detect any significant reduction in intracellular p24 expression in the DCs co-cultured with apoptotic activated CD4⁺ T cells after 72 hours. However, after 7 days of culture all eleven donors analyzed had a reduced frequency of p24⁺ DCs in the cultures containing apoptotic activated CD4⁺ T cells as compared to DCs exposed only to HIV-1_{BaL} (FIG. 20). These finding show that co-culture of DCs and apoptotic anti-CD3 and anti-CD28 activated CD4⁺ T cells results in an environment able to limit HIV-1 infection in DCs. There was no significant reduction in p24 expression in DCs after exposure to non-activated apoptotic T cells (FIG. 21).

To assess whether supernatant collected from the co-cultures with DCs and apoptotic activated T cells were able to induce maturation in DCs and reduce HIV-1 infected. We collected supernatant after 24 hours of co-culture and added different amounts to immature DCs. There was a significant induction of CD86 expression in the DCs exposed to a high dose supernatant (FIG. 22). In addition, there was a dose-response in terms of reduced p24 expression in DCs after exposure to the supernatant collected from co-cultures. Hence, we conclude that the reduction in p24 expression in DCs after co-culture with apoptotic activated T cells can at least in part be explained a soluble factor(s) released into the supernatant. Another explanation for the reduced infection rate in DCs could be down-regulation of the CCR5 receptor upon DC maturation.

We also performed kinetic experiments where DCs were first incubated with HIV-1_{BaL} for 30 min, 1 h or 2 h before addition of apoptotic activated T cells. We observed the same reduction in p24 expression even if the DCs were exposed to the virus for up to 2 h prior to addition of the apoptotic activated T cells (FIG. 237). Conversely, DCs were first exposed to apoptotic activated T cells for 30 min, 1 h or 2 h before addition of HIV-1_{Ba-L}. Again, we measured a similar reduced frequency of p24 expressing DCs even if the contact with the apoptotic activated T cells occurred for up to 2 h prior to HIV-1 exposure (FIG. 23).

The finding that interactions with DCs and apoptotic activated T cells (both non-infected and HIV-1 infected) leads to the formation of a anti-viral milieu has implications for the use of apoptotic HIV-1 carrying activated T cells as a therapeutic vaccine and for the use of apoptotic activated T cells as an additive to any therapeutic HIV-1 vaccine. It has been demonstrated that it is primarily HIV-1 specific T cells that get infected during HIV-1 infection. In addition, it was demonstrated that activated T cells are preferentially infected upon DC-T cell transmission. It is therefore a potential risk that HIV-1 specific T cells that are primed after a therapeutic immunization rapidly become infected. It would therefore be advantageous to achieve an anti-viral milieu at the site of DC-T cell interactions formed after immunization. Hence, the addition of apoptotic activated T cells to a vaccine composition has the potential of achieving an anti-viral effect in the DCs, which would reduce the risk of viral production in DCs and subsequent spread to T cells.

The finding that apoptotic activated T cells contacted with DCs leads to the formation of an anti-viral milieu also has implications for the development of a microbicide based on apoptotic activated T cells. Notably, it did not matter whether the DCs were exposed to the virus before or after addition of the apoptotic activated T cells to be able to detect the anti-viral effect. The kinetics investigated here were up to 2 h pre- or post-exposure. Hence, that would implicate the possibility to use a microbicide based on apoptotic activated T cells either pre- or post intercourse.

1. An adjuvant composition comprising a population of T cells, wherein the T cells are:
(a) activated; and
(b) apoptotic, or capable or being made apoptotic,
wherein the adjuvant composition is not itself a vaccine.

2. An adjuvant composition according to claim 1 comprising CD4⁺ T cells and/or CD8⁺ T cells.

3. An adjuvant composition according to claim 2 comprising PBMCs.

4. An adjuvant composition according to claim 1 comprising at least 50%, 60%, 70%, 80%, 90%, 95%, 96%, 97%, 98%, 99% or more CD 4⁺ T cells.

5. An adjuvant composition according to claim 1 comprising at least 50%, 60%, 70%, 80%, 90%, 95%, 96%, 97%, 98%, 99% or more CD 8+ T cells.

6. An adjuvant composition according to claim 1 wherein the T cells are isolated/derived from primary lymphocytes.

7. An adjuvant composition according to claim 1 wherein the T cells are derived from the subject in which the adjuvant composition is to be used.

8. An adjuvant composition according to claim 1 wherein the T cells are derived from the same species as that of the subject in which the adjuvant composition is to be used.

9. An adjuvant composition according to claim 1 wherein the T cells are activated by exposure to an activating agent selected from the group consisting of lectins, PHA, ConA, agents that induce Ca²⁺ influx in the T cells, ionomycin, alloantigens, superantigens, SEA, SEB, monoclonal antibodies, anti-CD3, anti-CD28, anti-CD49d, cytokines, IL-1, TNF-α, chemokines, chemokine receptors, and molecules capable of interfering with T cell surface receptors or their signal transducing molecules.

10. An adjuvant composition according to claim 9 wherein the activating agent is PHA.

11. An adjuvant composition according to claim 9 wherein the activating agent is an anti-CD3 antibody, and optionally, an anti-CD28 antibody.

12. An adjuvant composition according to claim 9 wherein the activating agent is an anti-CD49d antibody.

13. An adjuvant composition according to claim 1 wherein the CD4+ T cells are apoptotic, or capable or being made apoptotic, by exposure to an apoptosis-inducing agent selected from the group consisting of gamma-irradiation, cytostatic drugs, UV-irradiation, mitomycin C, starvation, serum deprivation, Fas ligation, cytokines, activators of cell death receptors, cell death receptor signal transducing molecules, growth factors, growth factor signal transducing molecules, cyclin interfering agents, agents which induce over-expression of oncogenes, molecules interfering with anti-apoptotic molecules, agents which alter the membrane potential of the mitochondria and steroids.

14. An adjuvant composition according to claim 13 wherein the apoptosis inducing agent is gamma-irradiation.

15. An adjuvant composition according to claim 1 wherein the adjuvant is for use with a vaccine against a pathogenic condition selected from the group consisting of HIV, tuberculosis, malaria, influenza and cancer.

16. An adjuvant composition according to claim 15 wherein the vaccine is an HIV vaccine.

17. An adjuvant composition according to claim 15 wherein the vaccine is a cancer vaccine.

18. An adjuvant composition according to claim 1 further comprising a vaccine wherein the vaccine comprises an attenuated or original viral vector selected from the group consisting of adenoviruses (such as adenoviruses 1, 2 and 5, chimpanzee), hepatitis viruses (such as hepatitis B virus and hepatitis C virus), Pox viruses (such as canarypox, vaccinia), rabies virus, murine leukaemia virus, alpha replicons, measles, rubella, polio, calicivirus, paramyxovirus, vesicular stomatitis virus, papilloma, leporipox, parvovirus, papovavirus, togavirus, picornavirus, reovirus and orthomyovirus (such as influenza viruses) and bacterial vectors (such as vectors selected from the group or mycobacteria, salmonella, listeria, Treponema pallidum, Neisseria gonorrhoeae, Chlamydia trachomatis and Haemophilus ducreyi).

19. An adjuvant composition according to claim 1 wherein the adjuvant composition further comprises a population of antigen-presenting cells.

20. An adjuvant composition according to claim 19 wherein the antigen presenting cells are macrophages.

21. An adjuvant composition according to claim 19 wherein the antigen presenting cells are dendritic cells.

22. An adjuvant composition according to claim 1, wherein the composition is frozen.

23. A pharmaceutical composition comprising an adjuvant composition according to claim 1 and a pharmaceutically acceptable carrier or diluent.

24. A combination product comprising:
(a) an adjuvant composition according to claim 1; and
(b) a vaccine,
wherein each or components (a) and (b) is formulated in admixture with a pharmaceutically-acceptable diluent or carrier said components (a) and (b) optionally being present in a form that is suitable for co-administration of each.

25. (canceled)

26. A kit comprising the combination product as claimed in claim 24.

27. A method of making an adjuvant composition according to claim 1, the method comprising obtaining a population of T cells, wherein the T cells are activated and apoptotic or capable or being made apoptotic.

28. A method according to claim 27 wherein the T cells are isolated/purified from primary lymphocytes.

29. A method according to claim 27 wherein the population of T cells in step (a) are derived from the subject in which the adjuvant composition to be used.

30. A method according to claim 27 wherein the population of T cells in step (a) are derived from the same species as that of the subject in which the adjuvant composition is to be used.

31. A method according to claim 27 further comprising the step of exposing the T cells to an activating agent.

32. A method according to claim 31 wherein the activating agent is selected from the group consisting of lectins PHA, ConA, agents that induce Ca²⁺ influx in the T cells, ionomycin, alloantigens, superantigens, SEA, SEB, monoclonal antibodies, anti-CD3, anti-CD28, anti-CD49d, cytokines, IL-1, TNF-α, chemokines, chemokine receptors, and molecules capable of interfering with T cell surface receptors or their signal transducing molecules.

33. A method according to claim 32 wherein the activating agent is PHA.

34. A method according to claim 32 wherein the activating agent is an anti-CD3 antibody and optionally comprises an anti-CD28 antibody.

35. A method according to claim 32 wherein the activating agent is an anti-CD49d antibody.

36. A method according to claim 27 further comprising the step of exposing the T cells to an apoptosis-inducing agent.

37. A method according to claim 36 wherein the apoptosis-inducing agent is selected from the group consisting of gamma-irradiation, cytostatic drugs, UV-irradiation, mitomycin C, starvation, serum deprivation, Fas ligation, cytokines, activators of cell death receptors, cell death receptor signal transducing molecules, growth factors, growth factor signal transducing molecules, cyclin interfering agents, agents which induce over-expression of oncogenes, molecules interfering with anti-apoptotic molecules, agents which alter the membrane potential of the mitochondria and steroids.

38. A method according to claim 37 wherein the apoptosis-inducing agent is gamma-irradiation.

39. A method according to claim 27 further comprising the step or culturing the T cells.

40. A method according to claim 27 further comprising the step of freezing the T cells.

41. A method according to claim 27 further comprising the step of adding a population of antigen-presenting cells to the T cells.

42. The method according to claim 41 wherein the antigen-presenting cells are macrophages.

43. The method according to claim 41 wherein the antigen-presenting cells are dendritic cells.

44-47. (canceled)

48. A method of activating antigen-presenting cells comprising contacting the antigen-presenting cells with an adjuvant composition according to claim 1 said composition optionally comprising a vaccine and a pharmaceutically acceptable carrier or diluent.
